(19) United States
(12) Patent Application Publication
(10) Pub. No.: US 2013/0109721 A1
(43) Pub. Date: May 2, 2013

(54) FAAH Inhibitors

(71) Applicants: Kevin Sprott, Needham, MA (US); John Jeffrey Talley, St. Louis, MO (US); Jane Yang, Boxborough, MA (US); Bo Peng, Arlington, MA (US)

(72) Inventors: Kevin Sprott, Needham, MA (US); John Jeffrey Talley, St. Louis, MO (US); Jane Yang, Boxborough, MA (US); Bo Peng, Arlington, MA (US)

(73) Assignee: Ironwood Pharmaceuticals, Inc., Cambridge, MA (US)

(21) Appl. No.: 13/514,343

(22) PCT Filed: Dec. 8, 2010
(86) PCT No.: PCT/US10/59428

§ 371 Date: Dec. 12, 2012

Related U.S. Application Data
(60) Provisional application No. 61/267,696, filed on Dec. 8, 2009.

(51) Int. Cl. C07D 401/12; A61K 31/403; C07D 495/04; A61K 45/06; A61K 31/4439; C07D 207/333; A61K 31/40; C07D 209/52; A61K 31/407

(52) U.S. Cl. CPC C07D 401/12; C07D 209/52; A61K 31/403; C07D 495/04; A61K 31/407; A61K 31/4439; C07D 207/333; A61K 31/40; A61K 45/06
USPC 514/338; 548/516; 514/412; 548/453; 546/276.7; 514/339; 546/278.1; 546/279.1; 514/343; 548/540; 514/423

(57) ABSTRACT

The present disclosure relates to N-benzyl pyrrole compounds of formula (I) useful as inhibitors of the enzyme Fatty Acid Amide Hydrolase (FAAH). The disclosure also provides pharmaceutically acceptable compositions comprising the compounds of the disclosure and methods of using the compositions in the treatment or prevention of various disorders.

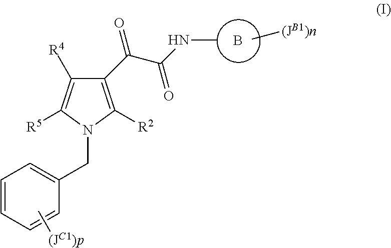

### PRIORITY CLAIM

This application claims priority to U.S. Provisional Application No. 61/267,696, filed on Dec. 8, 2009. The entire contents of the aforementioned application are incorporated herein by reference.

### TECHNICAL FIELD

The present disclosure relates to N-benzyl pyrrole compounds useful as inhibitors of the enzyme Fatty Acid Amide Hydrolase (FAAH). The disclosure also provides pharmaceutically acceptable compositions comprising the compounds of the disclosure and methods of using the compositions in the treatment of various disorders.

### BACKGROUND

The endocannabinoid (eCB) system has been implicated in a variety of processes including cell signaling, memory encoding, compensatory mechanisms, and immunosuppressant and anti-inflammatory responses. The eCB system comprises at least two receptors: the CB1 cannabinoid receptor, widely distributed in the brain and present in some peripheral organs, and the CB2 receptor, found principally in the periphery and immune systems and in some regions of the brain. The endogenous agonists of these receptors are the endogenous cannabinoids (eCBs), a family of lipids comprising the fatty acid Anandamide (AEA) as well as other fatty acids.

Endocannabinoid-degrading enzymes, including fatty acid amide hydrolase (FAAH), are responsible for cleaving and deactivating eCBs in vivo. FAAH is an integral membrane protein that is expressed in high levels in several brain regions, especially in the neurons of the hippocampus, cerebellum, neocortex and olfactory bulb. FAAH is the principal enzyme responsible for the hydrolysis of AEA in vivo and is also capable of hydrolyzing a wide variety of other substrates. It is known that inhibiting FAAH can lead to increases in fatty acids, including AEA, which could enhance cannabinoid signals within the eCB system. It has also been demonstrated that a number of fatty acid amides can induce analgesia in acute and chronic animal models of pain. Thus, increasing the level of AEA and other fatty acid amides (e.g., N-palmitoyl ethanolamide, N-oleoylethanol amide and oleamide) by inhibiting FAAH may lead to an increase in the nociceptive threshold. For these reasons, inhibitors of FAAH are useful in the treatment of pain. Inhibitors of FAAH might also be useful in the treatment of other disorders involving deregulation of the eCB system (e.g., anxiety, eating disorders, gastrointestinal and cardiovascular disorders, inflammation, excitotoxic insult, brain trauma and gastrointestinal diseases), and may avoid some of the side effects typically associated with CB receptor agonists (e.g., catalepsy or hypothermia).

In addition, there is evidence that when FAAH activity is reduced or absent, AEA acts as a substrate for COX-2, which can convert it to a prostamide. Thus, certain prostamides may be elevated in the presence of an FAAH inhibitor. Given that certain prostamides are associated with reduced intraocular pressure and ocular hypotensivity, FAAH inhibitors may also be useful agents for treating glaucoma.

### SUMMARY

The compounds of the instant disclosure, and their pharmaceutically acceptable salts thereof, are useful as FAAH inhibitors. They are represented by the general formula I,

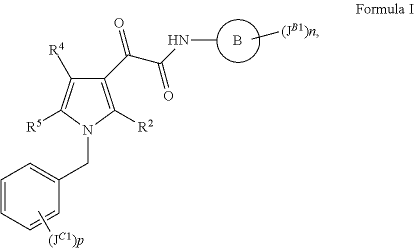

wherein:
ring B is selected from the group consisting of phenyl and a 5-6-membered monocyclic heteroaryl ring, wherein said monocyclic heteroaryl ring contains up to 3 ring heteroatoms selected from the group consisting of N, O and S;
n is an integer selected from the group consisting of 0, 1, 2 and 3;
each J^{B1} is independently selected from the group consisting of halogen, —NO₂, —CN, C₁₋₆ aliphatic, C₃₋₆ cycloaliphatic, C₁₋₆ haloaliphatic, C₁₋₆ alkoxy, C₁₋₆ haloalkoxy and C₃₋₆ cycloalkoxy;
each J^{C1} is independently selected from the group consisting of halogen, —NO₂, —CN, C₁₋₆ aliphatic, C₃₋₆ cycloaliphatic, C₁₋₆ haloaliphatic, C₁₋₆ alkoxy, C₁₋₆ haloalkoxy and C₃₋₆ cycloalkoxy;
p is an integer selected from the group consisting of 0, 1, 2 and 3;
R² is selected from the group consisting of halogen, —NO₂, —CN, C₁₋₆ aliphatic, phenyl, a 5-6-membered heteroaryl ring and a C₃₋₇ cycloalkyl, wherein said C₁₋₆ aliphatic, phenyl, 5-6-membered heteroaryl ring and C₃₋₇ cycloalkyl is optionally substituted by up to three instances of halogen;
R⁴ is selected from the group consisting of hydrogen, halogen, —CN, C₁₋₆ aliphatic, a C₃₋₇ cycloaliphatic ring, a 5-6-membered heteroaryl ring, phenyl, —OR^{Y} and —SR^{Y};
R⁵ is selected from the group consisting of hydrogen, halogen, —CN, C₁₋₆ aliphatic, a C₃₋₇ cycloaliphatic ring, a 5-6-membered heteroaryl ring, phenyl, —OR^{Y} and —SR^{Y}; wherein said C₁₋₆ aliphatic, C₃₋₇ cycloaliphatic ring, 5-6-membered heteroaryl ring, and phenyl is optionally substituted with up to three instances of halogen, C₁₋₄ alkyl, C₁₋₄ haloalkyl, C₁₋₄ alkoxy or C₁₋₄ haloalkoxy; or
R⁴ and R⁵, together with the two carbon atoms to which they are attached, form a C₅₋₈ cycloaliphatic ring, a 5-8-membered heterocyclic ring or a 5-membered heteroaryl ring; wherein said heterocyclic and heteroaryl ring formed by R⁴ and R⁵ contain up to three heteroatoms selected from the group consisting of N, O and S, and wherein said cycloaliphatic, heterocyclic and heteroaryl ring formed by R⁴ and R⁵ is optionally substituted by up to 3 instances of halogen, C₁₋₄ alkyl, C₁₋₄ haloalkyl, C₁₋₄ alkoxy or C₁₋₄ haloalkoxy; and
each R^{Y} is independently selected from the group consisting of C₁₋₆ aliphatic, C₃₋₇ cycloaliphatic, a 5-6-membered heteroaryl ring and phenyl, wherein each R^{Y} is optionally substituted by up to six instances of halogen, C₁₋₄ alkyl, C₁₋₄ haloalkyl, C₁₋₄ alkoxy or C₁₋₄ haloalkoxy;
   provided that the compound is not:

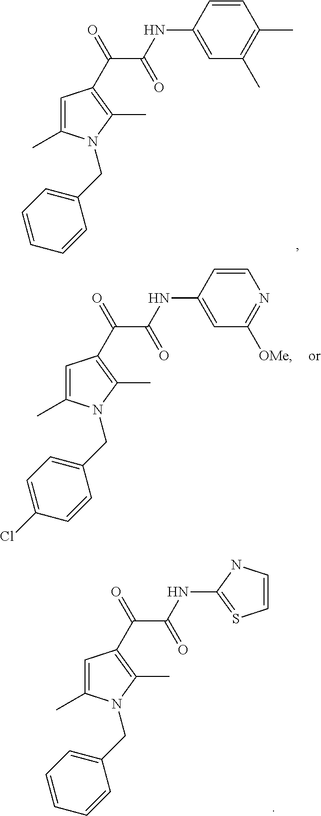

The invention also relates to pharmaceutical compositions comprising a compound according to formula I, or a pharmaceutically acceptable salt thereof, and a pharmaceutically acceptable carrier, vehicle or adjuvant. Also within the scope of the invention are pharmaceutical compositions further comprising at least one additional therapeutic agent.

The invention also relates to methods for the treatment or prevention of pain; autoimmune disorders; disease-states or indications that are accompanied by inflammatory processes; gastrointestinal diseases or disorders; pruritus; substance abuse related syndromes, disorders, diseases or withdrawal symptoms; psychiatric disorders; neurological or neurodegenerative disorders; ocular disorders; appetite-related disorders; gynecological disorders and sleep disorders.

### DETAILED DESCRIPTION

Reference will now be made in detail to certain embodiments of the invention, examples of which are illustrated in the accompanying structures and formulae. While the invention will be described in conjunction with the disclosed embodiments, it will be understood that they are not intended to limit the invention to those embodiments. Rather, the invention is intended to cover all alternatives, modifications and equivalents that may be included within the scope of the present invention as defined by the claims. The present invention is not limited to the methods and materials described herein but include any methods and materials similar or equivalent to those described herein that could be used in the practice of the present invention. In the event that one or more of the incorporated literature references, patents or similar materials differ from or contradict this application, including but not limited to defined terms, term usage, described techniques or the like, this application controls.

### DESCRIPTION OF EXEMPLARY COMPOUNDS

### Definitions and General Terminology

For purposes of this disclosure, the chemical elements are identified in accordance with the Periodic Table of the Elements, CAS version, and the Handbook of Chemistry and Physics, 75th Ed. 1994. Additionally, general principles of organic chemistry are described in Organic Chemistry, Thomas Sorrell, University Science Books, Sausalito: 1999, and March's Advanced Organic Chemistry, 5th Ed., Smith, M. B. and March, J., eds. John Wiley & Sons, New York: 2001, which are herein incorporated by reference in their entirety.

As described herein, compounds of the invention may optionally be substituted with one or more substituents, such as illustrated generally below, or as exemplified by particular classes, subclasses, and species of the invention. The phrase “optionally substituted” is used interchangeably with the phrase “substituted or unsubstituted.” In general, the term “substituted” refers to the replacement of one or more hydrogen radicals in a given structure with the radical of a specified substituent. Unless otherwise indicated, an optionally substituted group may have a substituent at each substitutable position of the group. When more than one position in a given structure can be substituted with more than one substituent selected from a specified group, the substituent may be either the same or different at each position. If a substituent radical or structure is not identified or defined as “optionally substituted”, the substituent radical or structure is not substituted. As it will be apparent to one of ordinary skill in the art, groups such as —H, halogen, —NO₂, —CN, —OH, —NH₂ or —OCF₃ would not be substitutable groups.

The phrase “up to”, as used herein, refers to zero or any integer number that is equal to or less than the number following the phrase. For example, optionally substituted with “up to 3” means substituted with 0, 1, 2, or 3 substituents. As described herein, a specified number range of atoms includes any integer therein. For example, a group having from 1-4 atoms could have 1, 2, 3 or 4 atoms. It will be understood by one of ordinary skill in the art that when a group is characterized as substituted (as opposed to optionally substituted) with, e.g., “up to 3” substituents, it can only be substituted with 1, 2 or 3 substituents.

When any variable occurs more than one time at any position, its definition on each occurrence is independent from every other occurrence.

Selection of substituents and combinations envisioned by this disclosure are only those that result in the formation of stable or chemically feasible compounds. Such choices and combinations will be apparent to those of ordinary skill in the art and may be determined without undue experimentation. The term “stable”, as used herein, refers to compounds that are not substantially altered when subjected to conditions that allow for their production, detection, and, in some embodiments, their recovery, purification, and use for one or more of the purposes disclosed herein. In some embodiments, a stable compound or chemically feasible compound is one that is not substantially altered when kept at a temperature of 25° C. or less, in the absence of moisture or other chemically reactive conditions, for at least a week.

A compound, such as the compounds of the invention or other compounds herein disclosed, may be present in its free form (e.g., an amorphous form or polymorphs). Under certain conditions, compounds may also form salts, and/or other multi-component crystalline forms (e.g., solvates (i.e., hydrates), and co-crystals). As used herein, the term co-form is synonymous with the term multi-component crystalline form. When one of the components in the co-form has clearly transferred a proton to the other component, the resulting co-form is referred to as a “salt”. When both compounds in a multi-component crystalline form are independently solids at room temperature, the resulting co-form is referred to as a “co-crystal”. In co-crystals no proton transfer takes place between the different components of the co-form. The formation of a salt or a co-crystal is determined by how large is the difference in the pKas between the partners that form the mixture. As used herein, a “solvate” refers to an association or complex of one or more solvent molecules and a compound disclosed herein (or its salts or co-crystals). A “hydrate” is a particular type of solvate in which the solvent is water. Examples of solvents that can form solvates include, but are not limited to: water, isopropanol, ethanol, methanol, dimethyl sulfoxide (DMSO), ethyl acetate, acetic acid, ethanolamine, tetrahydrofuran (THF), dichloromethane (DCM), N,N-dimethylformamide (DMF).

Unless only one of the isomers is drawn or named specifically, structures depicted herein are also meant to include all stereoisomeric (e.g., enantiomeric, diastereomeric, atropoisomeric and cis-trans isomeric) forms of the structure; for example, the R and S configurations for each asymmetric center, Ra and Sa configurations for each asymmetric axis, (Z) and (E) double bond configurations, and cis and trans conformational isomers. Therefore, single stereochemical isomers as well as racemates, and mixtures of enantiomers, diastereomers, and cis-trans isomers (double bond or conformational) of the present compounds are within the scope of the present disclosure. Unless otherwise stated, all tautomeric forms of the compounds of the present disclosure are within the scope of the disclosure.

The present disclosure also embraces isotopically labeled compounds that are identical to those recited herein, but for the fact that one or more atoms are replaced by an atom having an atomic mass or mass number different from the atomic mass or mass number usually found in nature. All isotopes of any particular atom or element as specified are contemplated within the scope of the compounds of the invention, and their uses. Exemplary isotopes that can be incorporated into compounds of the invention include isotopes of hydrogen, carbon, nitrogen, oxygen, phosphorus, sulfur, fluorine, chlorine, and iodine, such as ²H, ³, ¹¹C, ¹³C, ¹⁴C, ¹³N, ¹⁵N, ¹⁵O, ¹⁷O, ¹⁸O, ³²P, ³³P, ³⁵S, ¹⁸F, ³⁶Cl, ¹²³I, and ¹²⁵I, respectively. Certain isotopically labeled compounds of the present invention (e.g., those labeled with ³H and ¹⁴C) are useful in compound and/or substrate tissue distribution assays. Tritiated (i.e., ³H) and carbon-14 (i.e., ¹⁴C) isotopes are useful for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Positron-emitting isotopes such as ¹⁵O, ^{—}N, ¹¹C, and ¹⁸F are useful for positron emission tomography (PET) studies to examine substrate receptor occupancy. Isotopically labeled compounds of the present invention can generally be prepared by following procedures analogous to those disclosed in the Schemes and/or in the Examples herein, by substituting an isotopically labeled reagent for a non-isotopically labeled reagent.

The terms “aliphatic” or “aliphatic group”, as used herein, mean a straight-chain (i.e., unbranched) or branched, substituted or unsubstituted hydrocarbon chain that is completely saturated or that contains one or more units of unsaturation. Unless otherwise specified, aliphatic groups contain 1-20 aliphatic carbon atoms. In some embodiments, aliphatic groups contain 1-10 aliphatic carbon atoms. In other embodiments, aliphatic groups contain 1-8 aliphatic carbon atoms. In still other embodiments, aliphatic groups contain 1-6 aliphatic carbon atoms. In other embodiments, aliphatic groups contain 1-4 aliphatic carbon atoms and in yet other embodiments, aliphatic groups contain 1-3 aliphatic carbon atoms. Suitable aliphatic groups include, but are not limited to, linear or branched, substituted or unsubstituted alkyl, alkenyl, or alkynyl groups. Specific examples of aliphatic groups include, but are not limited to: methyl, ethyl, propyl, butyl, isopropyl, isobutyl, vinyl, sec-butyl, tert-butyl, butenyl, propargyl, acetylene and the like.

The term “alkyl”, as used herein, refers to a saturated linear or branched-chain monovalent hydrocarbon radical. Unless otherwise specified, an alkyl group contains 1-20 carbon atoms (e.g., 1-20 carbon atoms, 1-10 carbon atoms, 1-8 carbon atoms, 1-6 carbon atoms, 1-4 carbon atoms or 1-3 carbon atoms). Examples of alkyl groups include, but are not limited to, methyl, ethyl, n-propyl, isopropyl, n-butyl, isobutyl, s-butyl, t-butyl, pentyl, hexyl, heptyl, octyl and the like.

The term “alkenyl” refers to a linear or branched-chain monovalent hydrocarbon radical with at least one site of unsaturation, i.e., a carbon-carbon, sp² double bond, wherein the alkenyl radical includes radicals having “cis” and “trans” orientations, or alternatively, “E” and “Z” orientations. Unless otherwise specified, an alkenyl group contains 2-20 carbon atoms (e.g., 2-20 carbon atoms, 2-10 carbon atoms, 2-8 carbon atoms, 2-6 carbon atoms, 2-4 carbon atoms or 2-3 carbon atoms). Examples include, but are not limited to, vinyl, allyl and the like.

The term “alkynyl” refers to a linear or branched monovalent hydrocarbon radical with at least one site of unsaturation, i.e., a carbon-carbon sp triple bond. Unless otherwise specified, an alkynyl group contains 2-20 carbon atoms (e.g., 2-20 carbon atoms, 2-10 carbon atoms, 2-8 carbon atoms, 2-6 carbon atoms, 2-4 carbon atoms or 2-3 carbon atoms). Examples include, but are not limited to, ethynyl, propynyl, and the like.

The term “carbocyclic” refers to a ring system formed only by carbon and hydrogen atoms. Unless otherwise specified, throughout this disclosure, carbocycle is used as a synonym of “non-aromatic carbocycle” or “cycloaliphatic”). In some instances, the term can be used in the phrase “aromatic carbocycle”, and in this case it refers to an “aryl group” as defined below.

The term “cycloaliphatic” (or “non-aromatic carbocycle”, “non-aromatic carbocyclyl”, “non-aromatic carbocyclic”) refers to a cyclic hydrocarbon that is completely saturated or that contains one or more units of unsaturation but which is not aromatic, and which has a single point of attachment to the rest of the molecule. Unless otherwise specified, a cycloaliphatic group may be monocyclic, bicyclic, tricyclic, fused, Spiro or bridged. In one embodiment, the term “cycloaliphatic” refers to a monocyclic C₃-C₁₂ hydrocarbon or a bicyclic C₇-C₁₂ hydrocarbon. In some embodiments, any individual ring in a bicyclic or tricyclic ring system has 3-7 members. Suitable cycloaliphatic groups include, but are not limited to, cycloalkyl, cycloalkenyl, and cycloalkynyl. Examples of aliphatic groups include cyclopropyl, cyclobutyl, cyclopentyl, cyclopentenyl, cyclohexyl, cyclohexenyl, cycloheptyl, cycloheptenyl, norbornyl, cyclooctyl, cyclononyl, cyclodecyl, cycloundecyl, cyclododecyl, and the like.

The term “cycloaliphatic” also includes polycyclic ring systems in which the non-aromatic carbocyclic ring can be “fused” to one or more aromatic or non-aromatic carbocyclic or heterocyclic rings or combinations thereof, as long as the radical or point of attachment is on the non-aromatic carbocyclic ring.

The term “heterocycle” (or “heterocyclyl” or “heterocyclic), as used herein, refers to a ring system in which one or more ring members are an independently selected heteroatom, which is completely saturated or that contains one or more units of unsaturation but which is not aromatic, and which has a single point of attachment to the rest of the molecule. Unless otherwise specified, through this disclosure, heterocycle is used as a synonym of “non-aromatic heterocycle”). In some instances the term can be used in the phrase “aromatic heterocycle”, and in this case it refers to a “heteroaryl group” as defined below. The term heterocycle also includes fused, Spiro or bridged heterocyclic ring systems. Unless otherwise specified, a heterocycle may be monocyclic, bicyclic or tricyclic. In some embodiments, the heterocycle has 3-18 ring members in which one or more ring members is a heteroatom independently selected from oxygen, sulfur or nitrogen, and each ring in the system contains 3 to 7 ring members. In other embodiments, a heterocycle may be a monocycle having 3-7 ring members (2-6 carbon atoms and 1-4 heteroatoms) or a bicycle having 7-10 ring members (4-9 carbon atoms and 1-6 heteroatoms). Examples of bicyclic heterocyclic ring systems include, but are not limited to: adamantanyl, 2-oxa-bicyclo[2.2.2]octyl, 1-aza-bicyclo[2.2.2]octyl.

As used herein, the term “heterocycle” also includes polycyclic ring systems wherein the heterocyclic ring is fused with one or more aromatic or non-aromatic carbocyclic or heterocyclic rings, or with combinations thereof, as long as the radical or point of attachment is in the heterocyclic ring.

Examples of heterocyclic rings include, but are not limited to, the following monocycles: 2-tetrahydrofuranyl, 3-tetrahydrofuranyl, 2-tetrahydrothiophenyl, 3-tetrahydrothiophenyl, 2-morpholino, 3-morpholino, 4-morpholino, 2-thiomorpholino, 3-thiomorpholino, 4-thiomorpholino, 1-pyrrolidinyl, 2-pyrrolidinyl, 3-pyrrolidinyl, 1-tetrahydropiperazinyl, 2-tetrahydropiperazinyl, 3-tetrahydropiperazinyl, 1-piperidinyl, 2-piperidinyl, 3-piperidinyl, 1-pyrazolinyl, 3-pyrazolinyl, 4-pyrazolinyl, 5-pyrazolinyl, 1-piperidinyl, 2-piperidinyl, 3-piperidinyl, 4-piperidinyl, 2-thiazolidinyl, 3-thiazolidinyl, 4-thiazolidinyl, 1-imidazolidinyl, 2-imidazolidinyl, 4-imidazolidinyl, 5-imidazolidinyl; and the following bicycles: 3-1H-benzimidazol-2-one, 3-(1-alkyl)-benzimidazol-2-one, indolinyl, tetrahydroquinolinyl, tetrahydroisoquinolinyl, benzothiolane, benzodithiane, and 1,3-dihydro-imidazol-2-one.

As used herein, the term “aryl” (as in “aryl ring” or “aryl group”), used alone or as part of a larger moiety, as in “aralkyl”, “aralkoxy”, “aryloxyalkyl”, refers to a carbocyclic ring system wherein at least one ring in the system is aromatic and has a single point of attachment to the rest of the molecule. Unless otherwise specified, an aryl group may be monocyclic, bicyclic or tricyclic and contain 6-18 ring members. The term also includes polycyclic ring systems where the aryl ring is fused with one or more aromatic or non-aromatic carbocyclic or heterocyclic rings, or with combinations thereof, as long as the radical or point of attachment is in the aryl ring. Examples of aryl rings include, but are not limited to, phenyl, naphthyl, indanyl, indenyl, tetralin, fluorenyl, and anthracenyl. An optionally substituted “aralkyl” can be substituted on both the alkyl and the aryl portion. For instance, unless otherwise indicated, as used in this disclosure, an optionally substituted aralkyl is attached to the rest of the molecule through the alkyl chain and optionally substituted in the aryl portion. The same principle applies, for example, to a substituted aralkoxy, which would be attached to the rest of the molecule through the oxygen of the alkoxy and substituted on the aryl portion. A substituted aryloxyalkyl would be attached to the rest of the molecule through the alkyl chain and substituted on the aryl ring, which in turn would be attached to the alkyl chain through an oxygen atom.

The term “heteroaryl” (or “heteroaromatic” or “heteroaryl group” or “aromatic heterocycle”) used alone or as part of a larger moiety as in “heteroaralkyl” or “heteroarylalkoxy” refers to a ring system wherein at least one ring in the system is aromatic and contains one or more heteroatoms, wherein each ring in the system contains 3 to 7 ring members and which has a single point of attachment to the rest of the molecule. Unless otherwise specified, a heteroaryl ring system may be monocyclic, bicyclic or tricyclic and have a total of five to fourteen ring members. In one embodiment, all rings in a heteroaryl system are aromatic. Also included in this definition are heteroaryl radicals where the heteroaryl ring is fused with one or more aromatic or non-aromatic carbocyclic or heterocyclic rings, or combinations thereof, as long as the radical or point of attachment is in the heteroaryl ring. A bicyclic 6,5 heteroaromatic system, as used herein, for example, is a six-membered heteroaromatic ring fused to a second five-membered ring wherein the radical or point of attachment is on the six-membered ring.

Heteroaryl rings include, but are not limited to the following monocycles: 2-furanyl, 3-furanyl, N-imidazolyl, 2-imidazolyl, 4-imidazolyl, 5-imidazolyl, 3-isoxazolyl, 4-isoxazolyl, 5-isoxazolyl, 2-oxazolyl, 4-oxazolyl, 5-oxazolyl, N-pyrrolyl, 2-pyrrolyl, 3-pyrrolyl, 2-pyridyl, 3-pyridyl, 4-pyridyl, 2-pyrimidinyl, 4-pyrimidinyl, 5-pyrimidinyl, pyridazinyl (e.g., 3-pyridazinyl), 2-thiazolyl, 4-thiazolyl, 5-thiazolyl, tetrazolyl (e.g., 5-tetrazolyl), triazolyl (e.g., 2-triazolyl and 5-triazolyl), 2-thienyl, 3-thienyl, pyrazolyl (e.g., 2-pyrazolyl), isothiazolyl, 1,2,3-oxadiazolyl, 1,2,5-oxadiazolyl, 1,2,4-oxadiazolyl, 1,2,3-triazolyl, 1,2,3-thiadiazolyl, 1,3,4-thiadiazolyl, 1,2,5-thiadiazolyl, pyrazinyl, 1,3,5-triazinyl, and the following bicycles: benzimidazolyl, benzofuryl, benzothiophenyl, benzopyrazinyl, benzopyranonyl, indolyl (e.g., 2-indolyl), purinyl, quinolinyl (e.g., 2-quinolinyl, 3-quinolinyl, 4-quinolinyl), and isoquinolinyl (e.g., 1-isoquinolinyl, 3-isoquinolinyl, or 4-isoquinolinyl).

As used herein, “cyclo” (or “cyclic”, or “cyclic moiety”) encompasses mono-, bi- and tri-cyclic ring systems including cycloaliphatic, heterocyclic, aryl or heteroaryl, each of which has been previously defined.

“Fused” bicyclic ring systems comprise two rings which share two adjoining ring atoms.

“Bridged” bicyclic ring systems comprise two rings which share three or four adjacent ring atoms. As used herein, the term “bridge” refers to a bond or an atom or a chain of atoms connecting two different parts of a molecule. The two atoms that are connected through the bridge (usually but not always, two tertiary carbon atoms) are referred to as “bridgeheads”. Examples of bridged bicyclic ring systems include, but are not limited to, adamantanyl, norbornanyl, bicyclo[3.2.1]octyl, bicyclo[2.2.2]octyl, bicyclo[3.3.1]nonyl, bicyclo[3.2.3]nonyl, 2-oxa-bicyclo[2.2.2]octyl, 1-aza-bicyclo[2.2.2]octyl, 3-aza-bicyclo[3.2.1]octyl, and 2,6-dioxa-tricyclo[3.3.1.03,7]nonyl.

“Spiro” bicyclic ring systems share only one ring atom (usually a quaternary carbon atom).

The term “ring atom” refers to an atom such as C, N, O or S that is part of the ring of an aromatic group, a cycloaliphatic group or a heteroaryl ring. A “substitutable ring atom” is a ring carbon or nitrogen atom bonded to at least one hydrogen atom. The hydrogen can be optionally replaced with a suitable substituent group. Thus, the term “substitutable ring atom” does not include ring nitrogen or carbon atoms which are shared when two rings are fused. In addition, “substitutable ring atom” does not include ring carbon or nitrogen atoms when the structure depicts that they are already attached to one or more moiety other than hydrogen and no hydrogens are available for substitution.

“Heteroatom” refers to one or more of oxygen, sulfur, nitrogen, phosphorus, or silicon, including any oxidized form of nitrogen, sulfur, phosphorus, or silicon, the quaternized form of any basic nitrogen, or a substitutable nitrogen of a heterocyclic or heteroaryl ring, for example, N (as in 3,4-dihydro-2H-pyrrolyl), NH (as in pyrrolidinyl) or NR⁺ (as in N-substituted pyrrolidinyl).

In some embodiments, two independent occurrences of a variable may be taken together with the atom(s) to which each variable is bound to form a 5-8-membered, heterocyclyl, aryl, or heteroaryl ring or a 3-8-membered cycloalkyl ring. Exemplary rings that are formed when two independent occurrences of a substituent are taken together with the atom(s) to which each variable is bound include, but are not limited to the following: a) two independent occurrences of a substituent that are bound to the same atom and are taken together with that atom to form a ring, where both occurrences of the substituent are taken together with the atom to which they are bound to form a heterocyclyl, heteroaryl, carbocyclyl or aryl ring, wherein the group is attached to the rest of the molecule by a single point of attachment; and b) two independent occurrences of a substituent that are bound to different atoms and are taken together with both of those atoms to form a heterocyclyl, heteroaryl, carbocyclyl or aryl ring, wherein the ring that is formed has two points of attachment with the rest of the molecule. For example, where a phenyl group is substituted with two occurrences of Rₒ as in Formula D1:

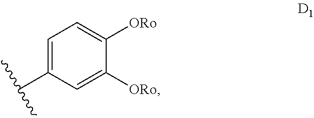

these two occurrences of Rₒ are taken together with the oxygen atoms to which they are bound to form a fused 6-membered oxygen containing ring as in Formula D2:

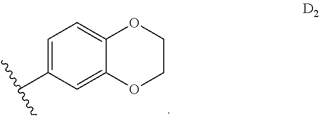 [SMILES: *c1ccc2c(c1)OCCO2]

It will be appreciated that a variety of other rings can be formed when two independent occurrences of a substituent are taken together with the atom(s) to which each substituent is bound and that the examples detailed above are not intended to be limiting.

In some embodiments, an alkyl or aliphatic chain can be optionally interrupted with another atom or group. This means that a methylene unit of the alkyl or aliphatic chain can optionally be replaced with said other atom or group. Unless otherwise specified, the optional replacements form a chemically stable compound. Optional interruptions can occur both within the chain and/or at either end of the chain; i.e. both at the point of attachment(s) to the rest of the molecule and/or at the terminal end. Two optional replacements can also be adjacent to each other within a chain so long as it results in a chemically stable compound. Unless otherwise specified, if the replacement or interruption occurs at a terminal end of the chain, the replacement atom is bound to an H on the terminal end. For example, if —CH₂CH₂CH₃ were optionally interrupted with —O—, the resulting compound could be —OCH₂CH₃, —CH₂OCH₃, or —CH₂CH₂OH. In another example, if the divalent linker —CH₂CH₂CH₂— were optionally interrupted with —O—, the resulting compound could be —OCH₂CH₂—, —CH₂OCH₂—, or —CH₂CH₂O—. The optional replacements can also completely replace all of the carbon atoms in a chain. For example, a C₃ aliphatic can be optionally replaced by —N(R^{$})—, —C(O)—, and —N(R^{$})— to form —N(R^{$})C(O)N(R^{$})— (a urea).

In general, the term “vicinal” refers to the placement of substituents on a group that includes two or more carbon atoms, wherein the substituents are attached to adjacent carbon atoms.

In general, the term “geminal” refers to the placement of substituents on a group that includes two or more carbon atoms, wherein the substituents are attached to the same carbon atom.

The terms “terminally” and “internally” refer to the location of a group within a substituent. A group is terminal when the group is present at the end of the substituent not further bonded to the rest of the chemical structure. Carboxyalkyl, i.e., R^{X}O(O)C-alkyl is an example of a carboxy group used terminally. A group is internal when the group is present in the middle of a substituent at the end of the substituent bound to the rest of the chemical structure. Alkylcarboxy (e.g., alkyl-C(O)O— or alkyl-O(CO)—) and alkylcarboxyaryl (e.g., alkyl-C(O)O-aryl- or alkyl-O(CO)-aryl-) are examples of carboxy groups used internally.

As described herein, a bond drawn from a substituent to the center of one ring within a multiple-ring system (as shown below), represents substitution of the substituent at any substitutable position in any of the rings within the multiple ring system. For example, formula D3 represents possible substitution in any of the positions shown in formula D4:

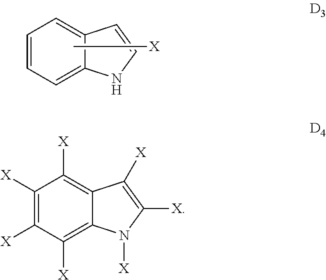

This also applies to multiple ring systems fused to optional ring systems (which would be represented by dotted lines). For example, in Formula D5, X is an optional substituent both for ring A and ring B.

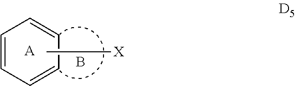

If, however, two rings in a multiple ring system each have different substituents drawn from the center of each ring, then, unless otherwise specified, each substituent only represents substitution on the ring to which it is attached. For example, in Formula D6, Y is an optional substituent for ring A only, and X is an optional substituent for ring B only.

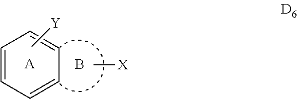

As used herein, the terms “alkoxy” or “alkylthio” refer to an alkyl group, as previously defined, attached to the molecule, or to another chain or ring, through an oxygen (“alkoxy,” e.g., —O-alkyl) or a sulfur (“alkylthio,” e.g., —S-alkyl) atom. The terms Cₙ₋ₘ “alkoxyalkyl”, Cₙ₋ₘ “alkoxyalkenyl”, Cₙ₋ₘ “alkoxyaliphatic”, and Cₙ₋ₘ “alkoxyalkoxy” mean alkyl, alkenyl, aliphatic or alkoxy, as the case may be, substituted with one or more alkoxy groups, wherein the total number of carbons between the alky and alkoxy, alkenyl and alkoxy, aliphatic and alkoxy or alkoxy and alkoxy, as the case may be, is between the values of n and m. When these moieties are optionally substituted they can be substituted in either of the portions on both sides of the oxygen or sulfur. For example, an optionally substituted C₄ alkoxyalkyl could be, for instance, —CH₂CH₂OCH₂(Me)CH₃ or —CH₂(OH)OCH₂CH₂CH₃; a C₅ alkoxyalkenyl could be, for instance, ═CHCH₂OCH₂CH₂CH₃ or ═CHCH₂CH₂OCH₂CH₃.

The terms “aryloxy”, “arylthio”, “benzyloxy” or “benzylthio”, refer to an aryl or benzyl group attached to the molecule, or to another chain or ring, through an oxygen (“aryloxy” “benzyloxy,” e.g., —O-Ph, —OCH₂Ph) or sulfur (“arylthio,” e.g., —S-Ph, —S—CH₂Ph) atom. For instance, the terms “aryloxyalkyl”, “benzyloxyalkyl” “aryloxyalkenyl” and “aryloxyaliphatic” mean alkyl, alkenyl or aliphatic, as the case may be, substituted with one or more aryloxy or benzyloxy groups, as the case may be. In this case, the number of atoms for each aryl, aryloxy, alkyl, alkenyl or aliphatic will be indicated separately. Thus, a 5-6-membered aryloxy(C₁₋₄alkyl) is a 5-6 membered aryl ring, attached via an oxygen atom to a C₁₋₄ alkyl chain, which, in turn, is attached to the rest of the molecule via the terminal carbon of the C₁₋₄ alkyl chain.

An optionally substituted “aralkyl” can potentially be substituted on both the alkyl and the aryl portion. Unless otherwise indicated, as used in this disclosure, an optionally substituted aralkyl is attached to the rest of the molecule through the alkyl chain and optionally substituted in the aryl portion. The same principle applies to, for example, substituted aralkoxy, which would be attached to the rest of the molecule through the oxygen of the alkoxy and substituted on the aryl portion. A substituted aryloxyalkyl would be attached to the rest of the molecule through the alkyl chain and substituted on the aryl ring, which in turn would be attached to the alkyl chain through an oxygen atom. For example, an optionally substituted 6-membered aryloxy(C₃alkyl) group could be, for instance, —(CH₃)₂CH₂— [p-(MeO)-Ph]; an optionally substituted 6-membered heteroaryloxy(C₄alkyl) could, for instance, be —CH₂CH₂CH₂—O-(3-F-2-pyrydyl) or —CH(CH₃)—O—CH₂CH₂-(5,6-dimethyl-1,3-pyrimidine). If the alkyl chain on the “aralkyl” group is also substituted that will be specifically indicated. For instance, an optionally substituted 6-membered heteroaryloxy(C₄alkyl) that is also optionally substituted on the alkyl, would be referred to as “an optionally substituted 6-membered heteroaryloxy(C₄alkyl), wherein said C₄ alkyl chain is optionally substituted”. An example of this latter group could be 5,6-dimethyl-1,3-pyrimidine-O—CF(CH₃)—CH(OH)CH₂—, wherein the alkyl chain is substituted with F and with —OH.

As used herein, the terms “halogen” or “halo” mean F, Cl, Br, or I.

The terms “haloalkyl”, “haloalkenyl”, “haloaliphatic”, and “haloalkoxy” mean alkyl, alkenyl, aliphatic or alkoxy, as the case may be, substituted with one or more halogen atoms. For example, a C₁₋₃ haloalkyl could be —CFHCH₂CHF₂ and a C₁₋₂ haloalkoxy could be —OC(Br)HCHF₂. This term includes perfluorinated alkyl groups, such as —CF₃ and —CF₂CF₃.

As used herein, the term “cyano” refers to —CN or —C≡N.

The terms “cyanoalkyl”, “cyanoalkenyl”, “cyanoaliphatic”, and “cyanoalkoxy” mean alkyl, alkenyl, aliphatic or alkoxy, as the case may be, substituted with one or more cyano groups. For example, a C₁₋₃ cyanoalkyl could be —C(CN)₂CH₂CH₃ and a C₁₋₂ cyanoalkenyl could be ═CHC(CN)H₂.

As used herein, an “amino” group refers to —NH₂.

The terms “aminoalkyl”, “aminoalkenyl”, “aminoaliphatic”, and “aminoalkoxy” mean alkyl, alkenyl, aliphatic or alkoxy, as the case may be, substituted with one or more amino groups. For example, a C₁₋₃ aminoalkyl could be —CH(NH₂)CH₂CH₂NH₂ and a C₁₋₂ aminoalkoxy could be —OCH₂CH₂NH₂.

The term “hydroxyl” or “hydroxy” refers to —OH.

The terms “hydroxyalkyl”, “hydroxyalkenyl”, “hydroxyaliphatic”, and “hydroxyalkoxy” mean alkyl, alkenyl, aliphatic or alkoxy, as the case may be, substituted with one or more —OH groups. For example, a C₁₋₃ hydroxyalkyl could be —CH₂(CH₂OH)CH₃ and a C₄ hydroxyalkoxy could be —OCH₂C(CH₃)(OH)CH₃.

As used herein, an “aroyl” or “heteroaroyl” refers to a —C(O)-aryl or a —C(O)-heteroaryl. The aryl and heteroaryl portion of the aroyl or heteroaroyl is optionally substituted as previously defined.

As used herein, a “carbonyl”, used alone or in connection with another group refers to —C(O)— or —C(O)H. For example, as used herein, an “alkoxycarbonyl,” refers to a group such as —C(O)O(alkyl).

As used herein, an “oxo” refers to ═O, wherein oxo is usually, but not always, attached to a carbon atom (e.g., it can also be attached to a sulfur atom forming a sulfoxide or a sulfone). An aliphatic chain can be optionally interrupted by a carbonyl group or can optionally be substituted by an oxo group, and both expressions refer to the same: e.g., —CH₂—C(O)—CH₃.

As used herein, in the context of resin chemistry (e.g., using solid resins or soluble resins or beads), the term “linker” refers to a bifunctional chemical moiety attaching a compound to a solid support or soluble support.

In all other situations, a “linker”, as used herein, refers to a divalent group in which the two free valences are on different atoms (e.g., carbon or heteroatom) or are on the same atom but can be substituted by two different substituents. For example, a methylene group can be a C₁ alkyl linker (—CH₂—), which can be substituted by two different groups, one for each of the free valences (e.g., as in Ph-CH₂-Ph, wherein methylene acts as a linker between two phenyl rings). Ethylene can be a C₂ alkyl linker (—CH₂CH₂—) wherein the two free valences are on different atoms. The amide group, for example, can act as a linker when placed in an internal position of a chain (e.g., —CONH—). A linker can be the result of interrupting an aliphatic chain by certain functional groups or of replacing methylene units on said chain by said functional groups. E.g., a linker can be a C₁₋₆ aliphatic chain in which up to two methylene units are substituted by —C(O)— or —NH— (as in —CH₂—NH—CH₂—C(O)—CH₂— or —CH₂—NH—C(O)—CH₂—). An alternative way to define the same —CH₂—NH—CH₂—C(O)—CH₂— and —CH₂—NH—C(O)—CH₂— groups is as a C₃ alkyl chain optionally interrupted by up to two —C(O)— or —NH— moieties. Cyclic groups can also form linkers: e.g., a 1,6-cyclohexanediyl can be a linker between two R groups, as in

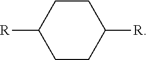

Divalent groups of the type ═CH—R or ═C—R₂, wherein both free valences are in the same atom and are attached the same substituent, are also possible. In this case, they will be referred to by their IUPAC accepted names. For instance, an alkylidene (such as, for example, a methylidene (═CH₂) or an ethylidene (═CH—CH₃)) would not be encompassed by the definition of a linker in this disclosure.

The term “protecting group”, as used herein, refers to an agent used to temporarily block one or more desired reactive sites in a multifunctional compound. In certain embodiments, a protecting group has one or more, or preferably all, of the following characteristics: a) reacts selectively in good yield to give a protected substrate that is stable to the reactions occurring at one or more of the other reactive sites; and b) is selectively removable in good yield by reagents that do not attack the regenerated functional group. Exemplary protecting groups are detailed in Greene, T. W., Wuts, P. G in Protective Groups in Organic Synthesis, Third Edition, John Wiley & Sons, New York: 1999, the entire contents of which are hereby incorporated by reference. The term “nitrogen protecting group”, as used herein, refers to an agents used to temporarily block one or more desired nitrogen reactive sites in a multifunctional compound. Preferred nitrogen protecting groups also possess the characteristics exemplified above, and certain exemplary nitrogen protecting groups are also detailed in Chapter 7 in Greene, T. W., Wuts, P. G in Protective Groups in Organic Synthesis, Third Edition, John Wiley & Sons, New York: 1999, the entire contents of which are hereby incorporated by reference.

As used herein, the term “displaceable moiety” or “leaving group” refers to a group that is associated with an aliphatic or aromatic group as defined herein and is subject to being displaced by nucleophilic attack by a nucleophile.

As used herein, “amide coupling agent” or “amide coupling reagent” means a compound that reacts with the hydroxyl moiety of a carboxy moiety thereby rendering it susceptible to nucleophilic attack. Exemplary amide coupling agents include DIC (diisopropylcarbodiimide), EDCI (1-Ethyl-3-(3-dimethylaminopropyl)carbodiimide), DCC (dicyclohexylcarbodiimide), BOP (Benzotriazol-1-yloxy-tris(dimethylamino)-phosphonium hexafluorophosphate), pyBOP ((Benzotriazol-1-yloxy)tripyrrolidinophosphonium Hexafluorophosphate), etc.

The compounds of the invention are defined herein by their chemical structures and/or chemical names. Where a compound is referred to by both a chemical structure and a chemical name, and the chemical structure and chemical name conflict, the chemical structure is determinative of the compound's identity.

In one aspect, the invention relates to a compound of Formula I, or a pharmaceutically acceptable salt thereof,

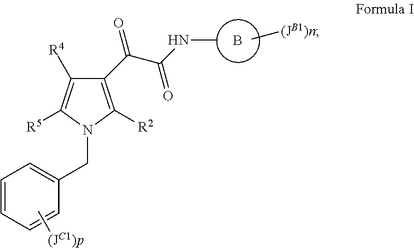

wherein:
ring B is selected from the group consisting of phenyl and a 5-6-membered monocyclic heteroaryl ring, wherein said monocyclic heteroaryl ring contains up to 3 ring heteroatoms selected from the group consisting of N, O and S;
n is an integer selected from the group consisting of 0, 1, 2 and 3;
each J^{B1} is independently selected from the group consisting of halogen, —NO₂, —CN, C₁₋₆ aliphatic, C₃₋₆ cycloaliphatic, C₁₋₆ haloaliphatic, C₁₋₆ alkoxy, C₁₋₆ haloalkoxy and C₃₋₆ cycloalkoxy;
each J^{C1} is independently selected from the group consisting of halogen, —NO₂, —CN, C₁₋₆ aliphatic, C₃₋₆ cycloaliphatic, C₁₋₆ haloaliphatic, C₁₋₆ alkoxy, C₁₋₆ haloalkoxy and C₃₋₆ cycloalkoxy;
p is an integer selected from the group consisting of 0, 1, 2 and 3;
R² is selected from the group consisting of halogen, —NO₂, —CN, C₁₋₆ aliphatic, phenyl, a 5-6-membered heteroaryl ring and a C₃₋₇ cycloalkyl, wherein said C₁₋₆ aliphatic, phenyl, 5-6-membered heteroaryl ring and C₃₋₇ cycloalkyl is optionally substituted by up to three instances of halogen;
R⁴ is selected from the group consisting of hydrogen, halogen, —CN, C₁₋₆ aliphatic, a C₃₋₇ cycloaliphatic ring, a 5-6-membered heteroaryl ring, phenyl, —OR^{Y} and —SR^{Y};
R⁵ is selected from the group consisting of hydrogen, halogen, —CN, C₁₋₆ aliphatic, a C₃₋₇ cycloaliphatic ring, a 5-6-membered heteroaryl ring, phenyl, —OR^{Y} and —SR^{Y}; wherein said C₁₋₆ aliphatic, C₃₋₇ cycloaliphatic ring, 5-6-membered heteroaryl ring, and phenyl is optionally substituted with up to three instances of halogen, C₁₋₄ alkyl, C₁₋₄ haloalkyl, C₁₋₄ alkoxy or C₁₋₄ haloalkoxy; or
R⁴ and R⁵, together with the two carbon atoms to which they are attached, form a C₅₋₈ cycloaliphatic ring, a 5-8-membered heterocyclic ring or a 5-membered heteroaryl ring; wherein said heterocyclic and heteroaryl ring formed by R⁴ and R⁵ contains up to three heteroatoms selected from the group consisting of N, O and S, and wherein said cycloaliphatic, heterocyclic and heteroaryl rings formed by R⁴ and R⁵ is optionally substituted by up to 3 instances of halogen, C₁₋₄ alkyl, C₁₋₄ haloalkyl, C₁₋₄ alkoxy or C₁₋₄ haloalkoxy; and
each R^{Y} is independently selected from the group consisting of C₁₋₆ aliphatic, C₃₋₇ cycloaliphatic, a 5-6-membered heteroaryl ring and phenyl, wherein each R^{Y} is optionally substituted by up to six instances of halogen, C₁₋₄ alkyl, C₁₋₄ haloalkyl, C₁₋₄ alkoxy or C₁₋₄ haloalkoxy;
   provided that the compound is not:

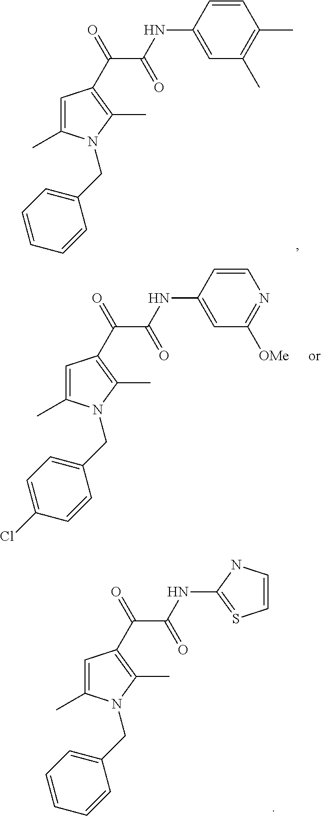

In some embodiments, ring B is an optionally substituted ring selected from the group consisting of phenyl, pyridine, pyrimidine, pyrazine, pyridazine, pyrrole, imidazole, pyrazole, furan, thiophene, triazole, tetrazole, thiazole, oxathiazole and oxazole.

In other embodiments, ring B is an optionally substituted pyridine or an optionally substituted phenyl. In still further embodiments, ring B is an optionally substituted pyridine. In other embodiments, ring B is an optionally substituted phenyl.

In some embodiments, n is selected from the group consisting of 0 and 1.

In some embodiments, each J^{B1} is independently selected from the group consisting of halogen, C₁₋₄ alkyl, cyclopropyl, cyclopropyloxy, C₁₋₄ haloalkyl, C₁₋₄ alkoxy and C₁₋₄ haloalkoxy.

In other embodiments, each J^{B1} is independently selected from the group consisting of halogen, methyl, ethyl, propyl, isopropyl, trifluoromethyl, methoxy, trifluoromethoxy, ethoxy, propyloxy and isopropyloxy.

In some embodiments, the moiety represented by

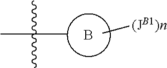

is selected from the group consisting of phenyl, 3-chlorophenyl, 3-pyridine, 4-pyridine and 3-methoxy-4-pyridine.

In some embodiments, p is selected from the group consisting of 0, 1 and 2.

In some embodiments, each J^{C1} is independently selected from the group consisting of halogen, C₁₋₄ alkyl, C₁₋₄ haloalkyl, cyclopropyl, cyclopropyloxy, C₁₋₄ alkoxy and C₁₋₄ haloalkoxy. In other embodiments, each J^{C1} is independently selected from the group consisting of halogen, methyl, ethyl, propyl, isopropyl, trifluoromethyl, methoxy, trifluoromethoxy, ethoxy, propyloxy and isopropyloxy. In still further embodiments, each J^{C1} is halogen. In still further embodiments, J^{C1} is chlorine and p is 1 or 2. In some embodiments, J^{C1} is fluorine and p is 1. In still other embodiments, J^{C1} is methoxy and p is 1.

In some embodiments, R² is selected from the group consisting of halogen, —NO₂, —CN, C₁₋₆ aliphatic and phenyl, wherein each C₁₋₆ aliphatic and phenyl is optionally substituted with up to three instances of halogen. In other embodiments, R² is methyl, ethyl, propyl, isopropyl, butyl, isobutyl, t-butyl, pentyl or hexyl. In still further embodiments, R² is methyl. In other embodiments, R² is phenyl.

In some embodiments, R⁴ is hydrogen, C₁₋₄ alkyl, a 5-6-membered heteroaryl or phenyl. In further embodiments, R⁴ is hydrogen. In other embodiments, R⁴ is phenyl.

In some embodiments, R⁵ is a C₁₋₄ alkyl, a 5-6-membered heteroaryl or phenyl. In further embodiments, R⁵ is methyl. In other embodiments, R⁵ is phenyl.

In some embodiments, R⁴ and R⁵, together with the two carbon atoms to which they are attached, form a C₅₋₈ cycloaliphatic ring, a 5-8-membered heterocyclic ring or a 5-membered heteroaryl ring, wherein said cycloaliphatic, heterocyclic and heteroaryl ring formed by R⁴ and R⁵ is optionally substituted with up to 3 instances of halogen, C₁₋₂ alkyl, C₁₋₂ haloalkyl, C₁₋₂ alkoxy or C₁₋₂ haloalkoxy. In further embodiments, R⁴ and R⁵, together with the two carbon atoms to which they are attached, form an optionally substituted C₅₋₈ cycloaliphatic ring. In other embodiments, R⁴ and R⁵, together with the two carbon atoms to which they are attached, form the fused ring:

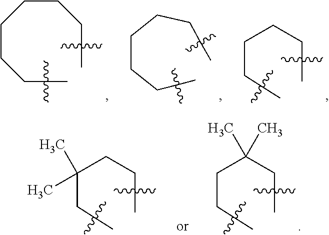

In other embodiments, R⁴ and R⁵, together with the two carbon atoms to which they are attached, form an optionally substituted 5-membered heteroaryl ring. In some other embodiments, R⁴ and R⁵, together with the two carbon atoms to which they are attached, form an optionally substituted thiophene ring. In some other embodiments, R⁴ and R⁵, together with the pyrrole ring to which they are attached and its substituents, form

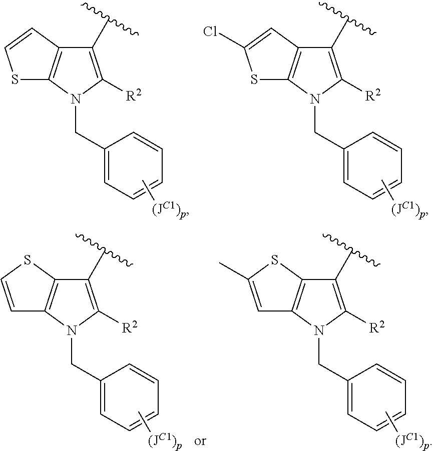

In another aspect, the invention relates to a compound of Formula II or a pharmaceutically acceptable salt thereof,

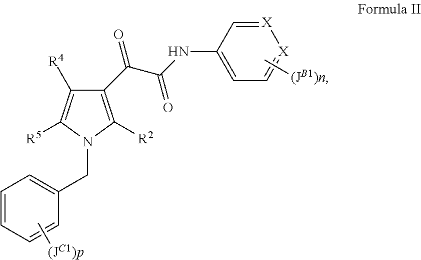

wherein each X is independently selected from the group consisting of C and N.

In another aspect, the invention relates to a compound of Formula III or a pharmaceutically acceptable salt thereof,

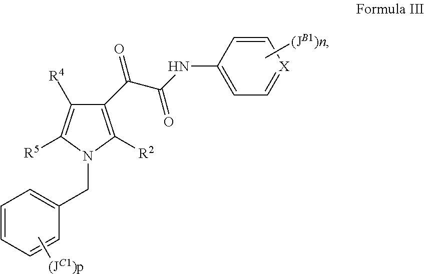

wherein: n is selected from the group consisting of 0 and 1 and wherein J^{B1} is selected from the group consisting of halogen and methoxy.

In another aspect, the invention relates to a compound of Formula IV or a pharmaceutically acceptable salt thereof,

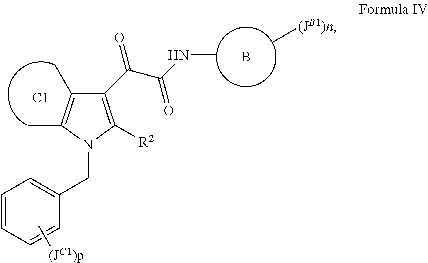

wherein ring C1 is an optionally substituted C₅₋₈ cycloaliphatic ring. In some embodiments, C1 is optionally substituted with up to two instances of methyl.

In another aspect, the invention relates to a compound of Formula V or a pharmaceutically acceptable salt thereof,

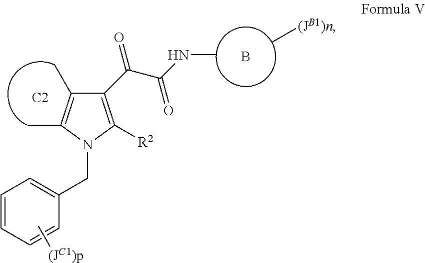

wherein ring C2 is an optionally substituted 5-membered heteroaryl ring. In other embodiments, C2 is an optionally substituted thiophene ring. In some other embodiments, C2 is optionally substituted with up to two instances of methyl or halogen.

In some embodiments, the compound is selected from the group consisting of:

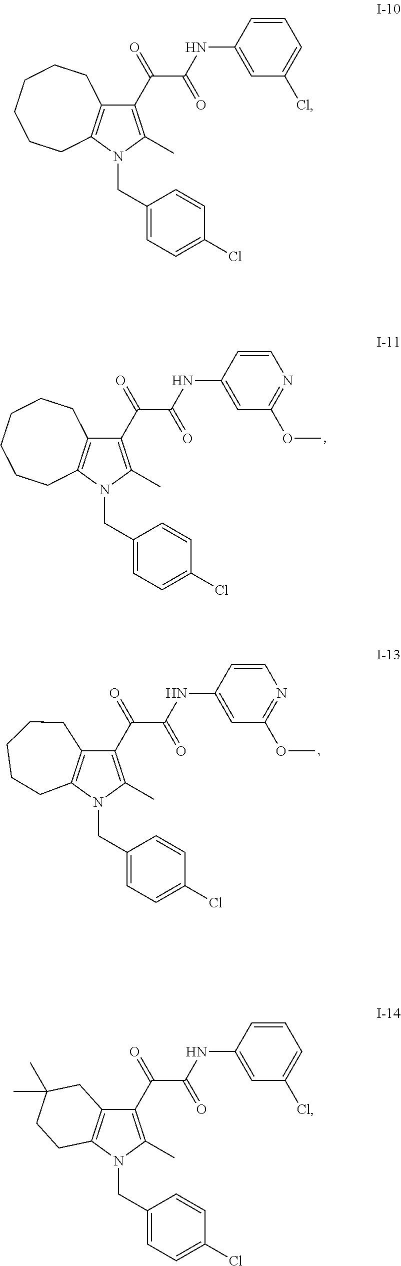

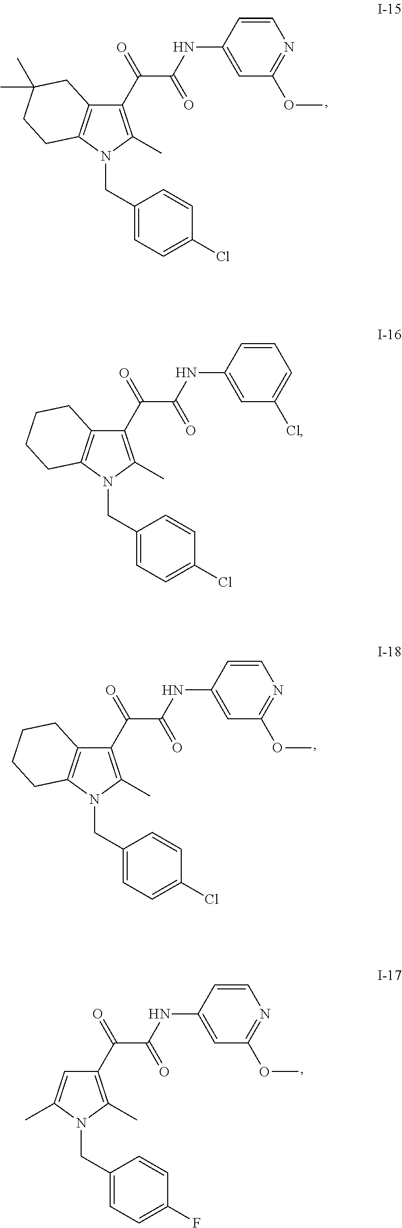

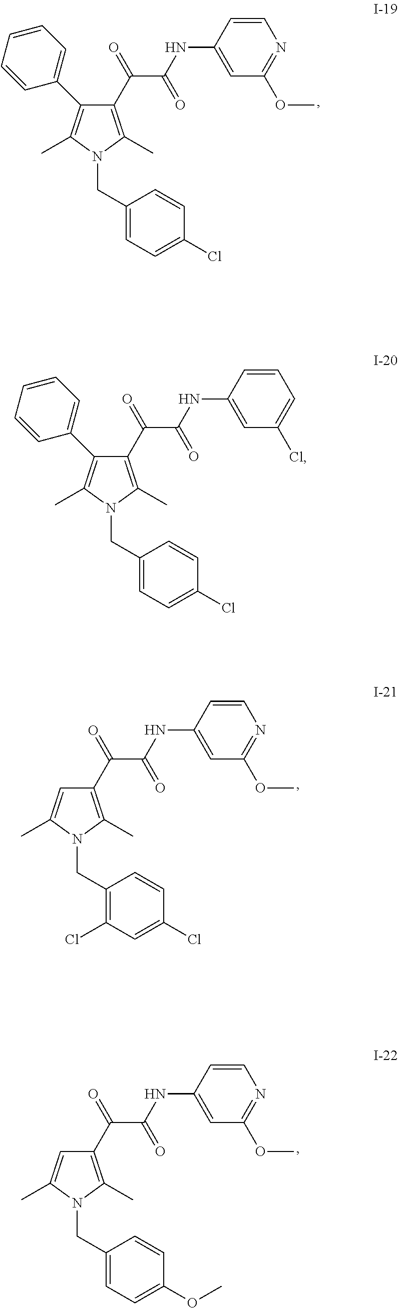

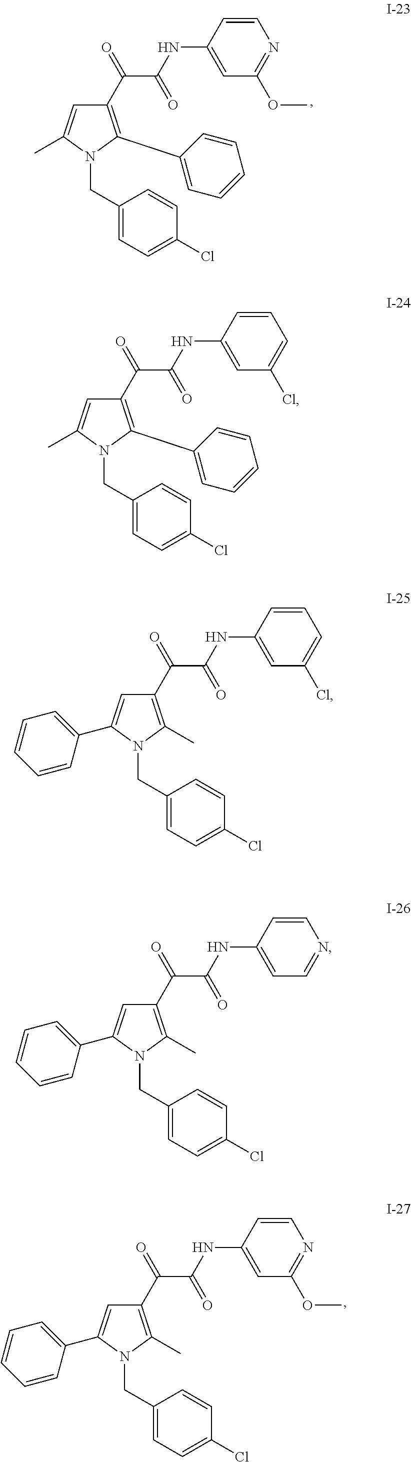

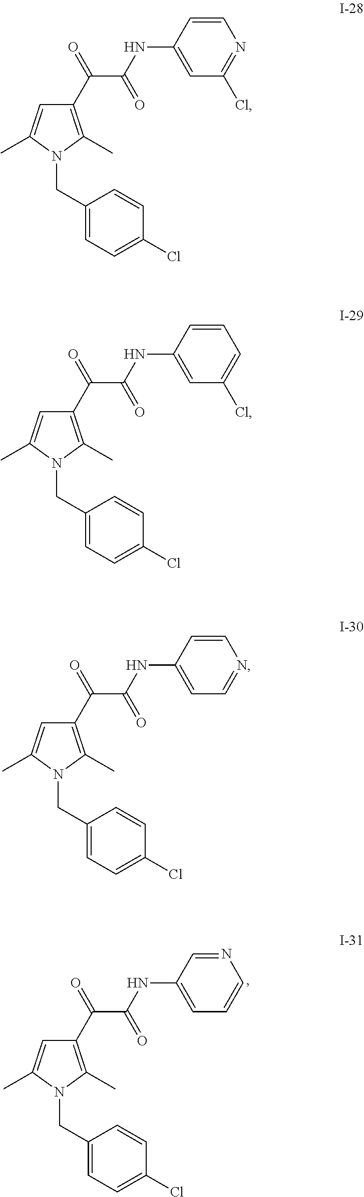 [SMILES: Cc1cc(C(=O)C(=O)Nc2ccnc(Cl)c2)c(C)n1Cc1ccc(Cl)cc1 ; Cc1cc(C(=O)C(=O)Nc2cccc(Cl)c2)c(C)n1Cc1ccc(Cl)cc1 ; Cc1cc(C(=O)C(=O)Nc2ccncc2)c(C)n1Cc1ccc(Cl)cc1 ; Cc1cc(C(=O)C(=O)Nc2cccnc2)c(C)n1Cc1ccc(Cl)cc1]

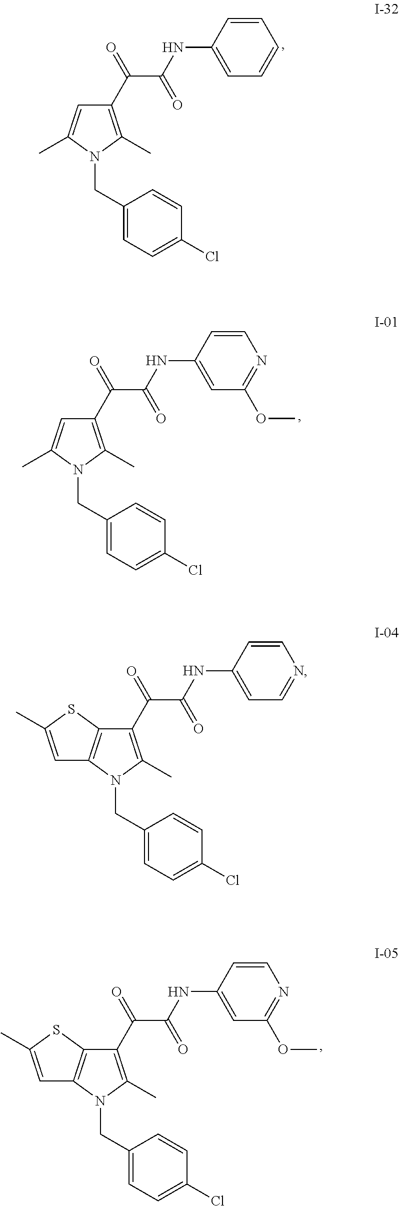

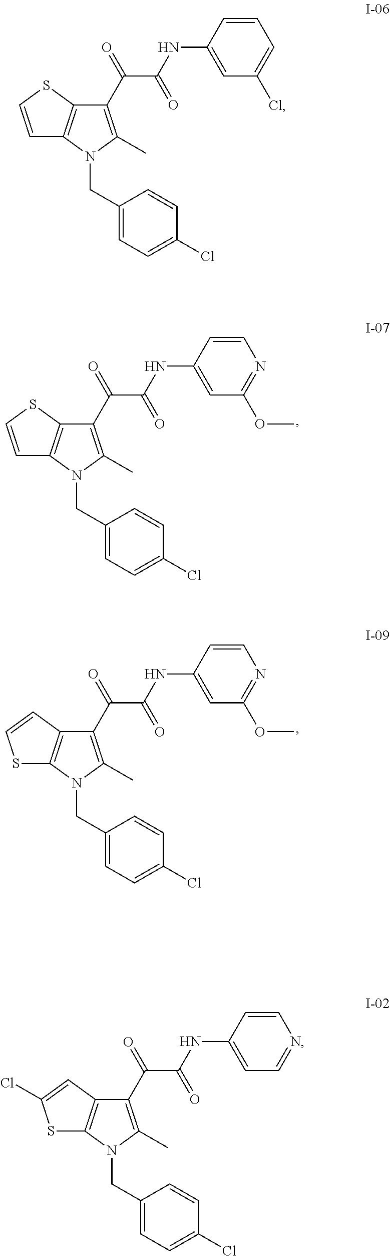 [SMILES: Cc1c(C(=O)C(=O)Nc2cccc(Cl)c2)c2sccc2n1Cc1ccc(Cl)cc1 ; COc1cc(NC(=O)C(=O)c2c(C)n(Cc3ccc(Cl)cc3)c3ccsc23)ccn1 ; COc1cc(NC(=O)C(=O)c2c(C)n(Cc3ccc(Cl)cc3)c3sccc23)ccn1 ; Cc1c(C(=O)C(=O)Nc2ccncc2)c2cc(Cl)sc2n1Cc1ccc(Cl)cc1]

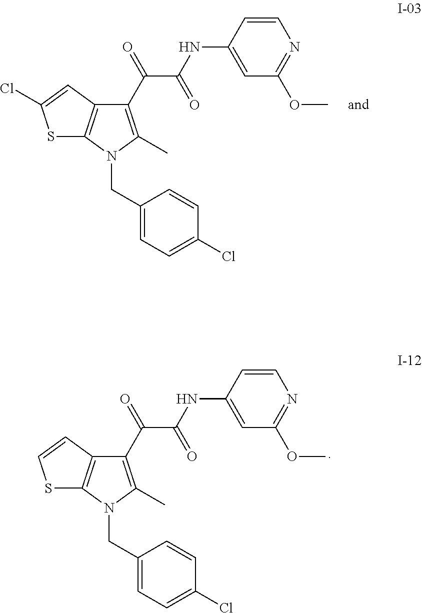 [SMILES: COc1cc(NC(=O)C(=O)c2c(C)n(Cc3ccc(Cl)cc3)c3sc(Cl)cc23)ccn1 ; COc1cc(NC(=O)C(=O)c2c(C)n(Cc3ccc(Cl)cc3)c3sccc23)ccn1]

In another aspect, the invention comprises a pharmaceutical composition comprising a compound discussed above, or a pharmaceutically acceptable salt thereof, and a pharmaceutically acceptable carrier, vehicle or adjuvant. In a further embodiment, the pharmaceutical composition further comprises at least one additional therapeutic agent.

In other embodiments, the pharmaceutical composition further comprises an additional therapeutic agent which is chosen from the group consisting of painkillers, non-steroidal anti-inflammatory drugs (NSAIDs), cannabinoid receptor agonists, opiate receptor agonists, anti-infective agents, sodium channel blockers, N-type calcium channel blockers, local anesthetics, VR1 agonists and antagonists, agents used for migraines, topical agents used in the treatment of localized pruritus, anti-inflammatory and/or immunosuppressive agents, agents designed to treat tobacco abuse (e.g., nicotine receptor partial agonists and nicotine replacement therapies), ADD/ADHD agents, agents to treat alcoholism, such as opioid antagonists, agents for reducing alcohol withdrawal symptoms such as benzodiazepines and beta-blockers, antihypertensive agents such as ACE inhibitors and Angiotensin II Receptor blockers, Renin inhibitors, vasodilators, agents used to treat glaucoma such as direct-acting Miotics (cholinergic agonists), indirect-acting Miotics (cholinesterase inhibitors), Carbonic anhydrase inhibitors, selective adrenergic agonists, Osmotic diuretics, antidepressants such as SSRIs, tricyclic antidepressants, and dopaminergic antidepressants, cognitive improvement agents, acetylcholinesterase inhibitors, anti-emetic agents (e.g., 5HT3 antagonists), neuroprotective agents, neuroprotective agents currently under investigation, antipsychotic medications, agents used for multiple sclerosis, disease-modifying anti-rheumatic drugs (DMARDS), biological response modifiers (BRMs), COX-2 selective inhibitors, COX-1 inhibitors, immunosuppressives, PDE4 inhibitors, corticosteroids, histamine H1 receptor antagonists, histamine H2 receptor antagonists, proton pump inhibitors, leukotriene antagonists, 5-lipoxygenase inhibitors, nicotinic acetylcholine receptor agonists, P2X3 receptor antagonists, NGF agonists and antagonists, NK1 and NK2 antagonists, NMDA antagonist, potassium channel modulators, GABA modulators, anti-cancer agents such as tyrosine kinase inhibitors, anti-hyperlipidemia drugs, appetite suppressing agents, anti-diabetic medications such as insulin, gastrointestinal (GI) agents, and serotonergic and noradrenergic modulators.

In another aspect, the invention provides a method for the treatment or prevention of pain comprising administering, alone or in combination therapy, to a patient in need thereof, a therapeutically or prophylactically acceptable dose of a pharmaceutical composition discussed above.

In some embodiments, the method of treatment for pain is selected from chronic pain, acute pain, perioperative pain (e.g., associated with surgery), postoperative pain, visceral pain, inflammatory pain, cancer pain, headache pain, pain associated with a cough, neuropathic pain, deafferentation pain, chronic nociceptive pain, dental pain (such as odontalgia), bone pain, joint pain (e.g., osteoarthritis or rheumatoid arthritis), myofascial pain (e.g., muscular injury, fibromyalgia), labor pain, pain associated with injuries, pain resulting from trauma, pain resulting from allergies, pain resulting from dermatitis, pain resulting from immunodeficiency, pain resulting from Hodgkin's disease, pain resulting from Myasthenia gravis, pain resulting from nephrotic syndrome, pain resulting from scleroderma, pain resulting from thyroiditis, central and peripheral pathway mediated pain, menstrual pain, neurogenic pain, dental pain, dysmenorrheal pain, visceral pain, neuropathic pain, post operative pain, headache, migraines, allodynia, hyperalgesia, post operative pain (e.g., associated with orthopedic surgery, gynecological surgery, abdominal surgery, incisions, or oral surgery), back pain, pain caused by inflammation (e.g., arthritis, osteoarthritis, spondylitis, rheumatoid arthritis, Crohn's disease and irritable bowel syndrome), and pain associated with injury, burns or trauma.

In some embodiments, a method is provided for the treatment or prevention of autoimmune disorders comprising administering, alone or in combination therapy, to a patient in need thereof a therapeutically or prophylactically acceptable dose of the pharmaceutical composition. In further embodiments, the autoimmune disorder is selected from the group consisting of alopecia greata (also known as systemic sclerosis (SS)), amyloses, amyotrophic lateral sclerosis, ankylosing spondylarthritis, ankylosing spondylitis, antiphospholipid syndrome, autoimmune Addison's disease, autoimmune hemolytic anemia, autoimmune hepatitis, autoimmune inner ear disease (AIED), autoimmune lymphoproliferative syndrome (ALPS), autoimmune thrombocytopenic purpura (ATP), Behcet's disease, cardiomyopathy, celiac sprue-dermatitis hepetiformis; chronic fatigue immune dysfunction syndrome (CFIDS), chronic inflammatory demyelinating polyneuropathy (CIPD), cicatricial pemphigold, cold agglutinin disease, connective tissue diseases, crest syndrome, Crohn's disease, Degos' disease, dermatomyositis-juvenile, discoid lupus, essential mixed cryoglobulinemia, fibromyalgia-fibromyositis, graft vs. host disease, transplantation rejection, Graves' disease, Guillain-Barre syndrome, Hashimoto's thyroiditis, idiopathic pulmonary fibrosis, idiopathic thrombocytopenia purpura (ITP), IgA nephropathy, insulin-dependent diabetes mellitus, juvenile chronic arthritis (Still's disease), juvenile rheumatoid arthritis, lupus erythematosus, Meniere's disease, multiple sclerosis, myasthenia gravis, pernicious anemia, polyarteritis nodosa, polychondritis, polyglandular syndromes, polymyalgia rheumatica, polymyositis and dermatomyositis, primary agammaglobulinemia, primary biliary cirrhosis, psoriasis, psoriatic arthritis, Raynaud's phenomena, rectional arthritis, Reiter's syndrome, rheumatic fever, rheumatoid arthritis, sarcoidosis, scleroderma (progressive systemic sclerosis (PSS)), Sjogren's syndrome, stiff-man syndrome, systemic lupus erythematosus, Takayasu arteritis, temporal arteritis/giant cell arteritis, ulcerative colitis, undifferentiated spondylarthritis, uveitis, vitiligo, and Wegener's granulomatosis.

In other embodiments, a method is provided for the treatment or prevention of disease states or indications that are accompanied by inflammatory processes, which comprises administering, alone or in combination therapy, to a patient in need thereof a therapeutically or prophylactically acceptable dose of a pharmaceutical composition discussed above. In further embodiments, the disease states or indications that are accompanied by inflammatory processes are chosen from the group consisting of: lung diseases such as asthma, bronchitis, allergic rhinitis, emphysema, adult respiratory distress syndrome (ARDS), pigeon fancier's disease, farmer's lung, chronic obstructive pulmonary disease (COPD), asthma including allergic asthma (atopic or non-atopic) as well as exercise-induced bronchoconstriction, occupational asthma, viral or bacterial exacerbation of asthma, other non-allergic asthmas and “wheezy-infant syndrome”, pneumoconiosis, including aluminosis, anthracosis, asbestosis, chalicosis, ptilosis, siderosis, silicosis, tabacosis and byssinosis; rheumatic diseases or autoimmune diseases or musculoskeletal diseases such as all forms of rheumatic diseases, especially rheumatoid arthritis, acute rheumatic fever, and polymyalgia rheumatica; reactive arthritis; rheumatic soft tissue diseases; inflammatory soft tissue diseases of other genesis; arthritic symptoms in degenerative joint diseases (arthroses); tendinitis, bursitis, osteoarthritis, traumatic arthritis, gout (metabolic arthritis); collagenoses of any genesis, e.g., systemic lupus erythematosus, scleroderma, polymyositis, dermatomyositis, Sjogren syndrome, Still disease, Felty syndrome; and osteoporosis and other bone resorption diseases; allergic diseases including all forms of allergic reactions, (e.g., allergic rhinitis, allergic conjunctivitis infectious parasitic, angioneurotic edema, hay fever, insect bites, allergic reactions to drugs, blood derivatives, contrast agents, etc.), anaphylactic shock (anaphylaxis), urticaria, angioneurotic edema, delayed or immediate hypersensitivity, and contact dermatitis; vascular diseases such as panarteritis nodosa, polyarteritis nodosa, periarteritis nodosa, arteritis temporalis, Wegner granulomatosis, giant cell arthritis, atherosclerosis, reperfusion injury, myocardial ischemia, thrombosis and erythema nodosum; dermatological diseases such as dermatitis, psoriasis, sunburn, burns, and eczema; renal, urinary and pancreatic diseases such as nephrotic syndrome and all types of nephritis (such as glomerulonephritis), pancreatitis, bladder hyperrelexia following bladder inflammation, urinary incontinence or vesicle inflammation, uresesthesia urgency, overactive bladder, urinary frequency, interstitial cystitis or chronic prostatitis; hepatic diseases such as acute liver cell disintegration; acute hepatitis of various genesis (such as viral, toxic, drug-induced) and chronically aggressive and/or chronically intermittent hepatitis, liver fibrosis associated with liver injury or disease, including fibrosis caused or exacerbated by alcoholic liver cirrhosis, chronic viral hepatitis, non-alcoholic steatohepatitis and primary liver cancer; gastrointestinal diseases such as ulcers, inflammatory bowel diseases, regional enteritis (Crohn's disease), ulcerated colitis, gastritis, aphthous ulcer, celiac disease, regional ileitis, ileus, esophagitis, NSAID-induced ulcer, non-ulcerative dyspepsia and gastroesophageal reflux disease; neurodegenerative diseases such as the neurodegeneration following stroke, cardiac arrest, pulmonary bypass, traumatic brain injury, cerebral ischemia, seizures, spinal cord injury or the like; eye diseases such as allergic keratitis, uveitis, or iritis, conjunctivitis, blepharitis, neuritis nervi optici, choroiditis, glaucoma and sympathetic ophthalmia; diseases of the ear, nose, and throat (ENT) area such as tinnitus, allergic rhinitis or hay fever, gingivitis, otitis externa, caused by contact eczema, infection, etc., and otitis media; progressive central nervous system or neurological diseases such as brain edema, particularly tumor-related brain edema, multiple sclerosis, acute encephalomyelitis, meningitis, acute spinal cord injury, trauma, dementia, particularly degenerative dementia (including senile dementia, Alzheimer's disease, Parkinson's disease and Creutzfeldt-Jacob disease, Huntington's chorea, Pick's disease, amyotrophic lateral sclerosis (ALS)), vascular dementia (including multi-infarct dementia and dementia associated with intracranial space—occupying lesions, infections and related conditions such as HIV infection), Guillain-Barre syndrome, myasthenia gravis, stroke, and various forms of seizures (such as nodding spasms); blood diseases such as acquired hemolytic anemia, aplastic anemia, and idiopathic thrombocytopenia; tumor diseases such as acute lymphatic leukemia, Hodgkin's disease, malignant lymphoma, lymphogranulomatoses, lymphosarcoma, solid malignant tumors, colorectal polyps, extensive metastases and other proliferative disorders such as diabetic retinopathy and tumor angiogenesis (e.g., wet macular degeneration); endocrine diseases such as endocrine opthalmopathy, endocrine orbitopathia, thyrotoxic crisis, Thyroiditis de Quervain, Hashimoto thyroiditis, Morbus Basedow, granulomatous thyroiditis, struma lymphomatosa, Graves disease, type I diabetes (such as insulin-dependent diabetes); organ and tissue transplantations and graft-versus-host diseases; severe states of shock such as septic shock, anaphylactic shock, and systemic inflammatory response syndrome (SIRS); viral or bacterial parasitic infectious disease: for example AIDS and meningitis; and various other disease states or conditions including, restenosis following percutaneous transluminal coronary angioplasty, acute and chronic pain, atherosclerosis, reperfusion injury, congestive heart failure, myocardial infarction, thermal injury, multiple organ injury secondary to trauma, necrotizing enterocolitis and syndromes associated with hemodialysis, leukopheresis, granulocyte transfusion, sarcoidosis, gingivitis, pyrexia, Edema resulting from trauma associated with burns, sprains or fracture, cerebral edema and angioedema, and diabetes (such as diabetic vasculopathy, diabetic neuropathy, diabetic retinopathy, post capillary resistance and diabetic symptoms associated with insulitis (e.g., Hyperglycemia, diuresis, proteinuria and increased nitrite and kallikrein urinary excretion)).

In some embodiments, a method is proved for the treatment or prevention of gastrointestinal diseases or disorders comprises administering, alone or in combination therapy, to a patient in need thereof a therapeutically or prophylactically acceptable dose of a pharmaceutical composition discussed above. In further embodiments, the gastrointestinal disease or disorder is chosen from the group consisting of: functional gastrointestinal disorders, ulcers, inflammatory bowel diseases (IBD), regional enteritis (Crohn's disease), ulcerative colitis, diarrhea, gastritis, aphthous ulcer, celiac disease, regional ileitis, ileus, functional dyspepsia, diverticulitis, gastrointestinal bleeding, irritable bowel syndrome (IBS), non-ulcerative dyspepsia and gastroesophageal reflux disease.

In some embodiments, a method is provided for the treatment or prevention of pruritus, comprises administering, alone or in combination therapy, to a patient in need thereof a therapeutically or prophylactically acceptable dose of a pharmaceutical composition discussed above. In further embodiments, the pruritus is dermal pruritus, neuropathic pruritus, neurogenic pruritus or psychogenic pruritus.

In some embodiments, a method is provided for the treatment or prevention of substance abuse-related syndromes, disorders, diseases or withdrawal symptoms comprising administering, alone or in combination therapy, to a patient in need thereof a therapeutically or prophylactically acceptable dose of a pharmaceutical composition discussed above. In further embodiments, the method of treating substance abuse-related syndromes, disorders, diseases or withdrawal symptoms are chosen from the group consisting of drug abuse and drug withdrawal, wherein the abused substances include alcohol, amphetamines, amphetamine-like substances, caffeine, cannabis, cocaine, hallucinogens, inhalants, opioids, nicotine (and/or tobacco products), heroin abuse, barbiturates, phencyclidine (or phencyclidine-like compounds), sedative-hypnotics, benzodiazepines, or combinations of any of the foregoing and the withdrawal symptoms include tobacco craving or nicotine dependency, addiction, or withdrawal,

In some embodiments, a method is provided for the treatment or prevention of psychiatric, disorders comprises administering, alone or in combination therapy, to a patient in need thereof a therapeutically or prophylactically acceptable dose of a pharmaceutical composition as discussed herein. In further embodiments, the psychiatric disorders are chosen from the group consisting of depressions (including major depressive disorder, bipolar depression, unipolar depression, single or recurrent major depressive episodes (e.g., with or without psychotic features, catatonic features, and/or melancholic features), postpartum onset, seasonal affective disorder, dysthymic disorders (e.g., with early or late onset and with or without atypical features), neurotic, depression and social phobia, depression accompanying dementia, anxiety, psychosis, social affective disorders, and/or cognitive disorders), manic-depressive, psychoses, bipolar disorders, extreme psychotic states such as mania, schizophrenia, and excessive mood swings where behavioral stabilization is desired), post-traumatic stress disorder, panic disorder, obsessive compulsive disorder, psychiatric tremors such as dyskinesias, dystonia and spasticity, attention disorders such as ADHD (attention deficit hyperactivity disorders), autism, anxiety states, generalized anxiety, agoraphobia, as well as those behavioral states characterized by social withdrawal.

In sonic embodiments, a method is provided for the treatment or prevention of neurological or neurodegenerative disorders, which comprises administering, alone or in combination therapy, to a patient in need thereof a therapeutically or prophylactically acceptable dose of a pharmaceutical composition discussed herein. In further embodiments, the neurological or neurodegenerative disorders are chosen from the group consisting of dementia, particularly degenerative dementia (including senile dementia, Alzheimer's disease, Pick's disease, Huntingdon's chorea, Parkinson's disease, prion disease and Creutzfeldt-Jakob disease and motor neuron disease); vascular dementia (including multi-infarct dementia); dementia associated with intracranial space occupying lesions; trauma; infections and related conditions (including HIV infection); dementia in Parkinson's disease; metabolism; toxins; anoxia and vitamin deficiency; and mild cognitive impairment associated with aging, particularly Age-Associated Memory Impairment; amyotrophic lateral sclerosis (ALS), multiple sclerosis, epilepsy, ischemia, traumatic head or brain injury, brain inflammation, eye injury, stroke and neuroinflammation; and neurodegeneration or decreased brain activity associated with stroke, cardiac arrest, pulmonary bypass, traumatic brain injury, hypoxia, hypoglycemia, gas poisoning, drug intoxication, diabetes mellitus, edema, spinal cord injury, cerebral ischemia, cerebral infarction, cerebral hemorrhage, subarachnoid hemorrhage, seizures or the like.

In some embodiments, a method is provided for the treatment or prevention of ocular disorders comprising administering, alone or in combination therapy, to a patient in need thereof a therapeutically or prophylactically acceptable dose of a pharmaceutical composition discussed above. In further embodiments, the ocular disorder is chosen from the group consisting of glaucoma (such as normal tension glaucoma), glaucoma-associated intraocular pressure retinitis, retinopathies, uveitis, acute injury to the eye tissue (e.g., conjunctivitis), high intraocular pressure, family history of glaucoma, glaucoma in the contralateral eye and high myopia.

In some embodiments, a method is provided for the treatment or prevention of appetite-related disorders comprising administering, alone or in combination therapy, to a patient in need thereof a therapeutically or prophylactically acceptable dose of a pharmaceutical composition discussed above. In further embodiments, the appetite-related disorder is chosen from the group consisting of: emesis, vomiting and nausea, food behavioral problems or feeding disorders such as anorexias, cachexias, wasting conditions and bulimia; and obesity or obesity-related disorders such as diabetes type II, hyperlipidemia.

In some embodiments, a method is provided for the treatment or prevention of gynecological disorders comprising administering, alone or in combination, a therapeutically acceptable dose of a pharmaceutical composition discussed above. In further embodiments, the gynecological disorder is uterus contraction caused by hormones, or prostanoid-induced muscle contraction such as premature labor, menstrual cramps, menstrual irregularity or dysmenorrhea.

In some embodiments, a method is provided for the treatment or prevention of sleep disorders comprising administering, alone or in combination, a pharmaceutically acceptable dose of a pharmaceutical composition discussed herein. In further embodiments, the sleep disorder is chosen from the group consisting of: insomnia, night terrors, bruxism, somnambulism, narcolepsy, circadian rhythm adjustment disorders, and sleep disorders associated with neurological or mental disorders or with pain.

In some embodiments, the methods above are provided for the treatment of a human patient. In other embodiments, the patient is a companion animal, exotic animal or a farm animal such as a dog, cat, mouse, rat, hamster, gerbil, guinea pig, rabbit, horse, pig or cow.

In some embodiments, a method is provided for inhibiting FAAH in a biological sample, comprising contacting said biological sample with a composition discussed herein.

### Pharmaceutically Acceptable Salts, Co-Forms and Pro-Drugs:

The phrase “pharmaceutically acceptable salt,” as used herein, refers to pharmaceutically acceptable organic or inorganic salts of a compound of Formula I. For use in medicine, the salts of the compounds of Formula I will be pharmaceutically acceptable salts. Other salts may, however, be useful in the preparation of the compounds of Formula I or of their pharmaceutically acceptable salts. A pharmaceutically acceptable salt may involve the inclusion of another molecule such as an acetate ion, a succinate ion or other counter ion. The counter ion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counter ion.

Pharmaceutically acceptable salts of the compounds described herein include those derived from suitable inorganic and organic acids and bases. In some embodiments, the salts can be prepared in situ during the final isolation and purification of the compounds. In other embodiments the salts can be prepared from the free form of the compound in a separate synthetic step.

When the compound of Formula I is acidic or contains a sufficiently acidic bioisostere, suitable pharmaceutically acceptable salts are salts prepared from pharmaceutically acceptable non-toxic bases including inorganic bases and organic bases. Salts derived from inorganic bases include aluminum, ammonium, calcium, copper, ferric, ferrous, lithium, magnesium, manganic salts, manganous, potassium, sodium, zinc and the like. Particular embodiments include ammonium, calcium, magnesium, potassium and sodium salts. Salts derived from pharmaceutically acceptable organic non-toxic bases include salts of primary, secondary and tertiary amines, substituted amines including naturally occurring substituted amines, cyclic amines and basic ion exchange resins, such as arginine, betaine, caffeine, choline, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine tripropylamine, tromethamine and the like.

When the compound of Formula I is basic or contains a sufficiently basic bioisostere, salts may be prepared from pharmaceutically acceptable non-toxic acids, including inorganic and organic acids. Such acids include acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid and the like. Particular embodiments include citric, hydrobromic, hydrochloric, maleic, phosphoric, sulfuric and tartaric acids. Other exemplary salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate, ethanesulfonate, benzenesulfonate, p-toluenesulfonate, and pamoate (i.e., 1,1′-methylene-bis-(2-hydroxy-3-naphthoate)) salts.

The preparation of the pharmaceutically acceptable salts described above and other typical pharmaceutically acceptable salts is more fully described by Berg et al., “Pharmaceutical Salts,” J. Pharm. Sci., 1977:66:1-19, incorporated herein by reference in its entirety.

In addition to the compounds described herein and their pharmaceutically acceptable salts, pharmaceutically acceptable solvates (e.g., hydrates) and co-crystals of these compounds and salts may also be employed in compositions to treat or prevent the disorders identified herein.

As used herein, the term “pharmaceutically acceptable solvate,” is a solvate formed from the association of one or more pharmaceutically acceptable solvent molecules to one of the compounds described herein. As used herein, the term “hydrate” means a compound described herein or a salt thereof that further includes a stoichiometric or non-stoichiometric amount of water bound by non-covalent intermolecular forces. The term solvate includes hydrates (e.g., hemihydrate, monohydrate, dihydrate, trihydrate, tetrahydrate, and the like).

“Pharmaceutically acceptable co-crystals” result when a pharmaceutically active compound crystallizes with another material (e.g., a carboxylic acid, a 4,4′-bipyridine or an excipient) that is also a solid at room temperature. Some pharmaceutically acceptable excipients are described in the next section. Other pharmaceutically acceptable substances that can be used to form co-crystals are exemplified by the GRAS (Generally regarded as safe) list of the U.S. Food and Drug Administration (FDA).

In addition to the compounds described herein, pharmaceutically acceptable pro-drugs of these compounds may also be employed in compositions to treat or prevent the disorders identified herein.

A “pharmaceutically acceptable pro-drug” includes any pharmaceutically acceptable ester, salt of an ester or other derivative or salt thereof of a compound described herein which, upon administration to a recipient, is capable of providing, either directly or indirectly, a compound described herein. Particularly favored pro-drugs are those that increase the bioavailability of the compounds when such compounds are administered to a patient (e.g., by allowing an orally administered compound to be more readily absorbed into the blood) or which enhance delivery of the parent compound to a biological compartment (e.g., the brain or lymphatic system) relative to the parent species. The term “pro-drug” encompasses a derivative of a compound that can hydrolyze, oxidize, or otherwise react under biological conditions (in vitro or in vivo) to provide a compound described herein. Examples of pro-drugs contemplated in this invention include, but are not limited to, analogs or derivatives of compounds of the invention that comprise biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. Other examples of pro-drugs include derivatives of compounds described herein that comprise —NO, —NO₂, —ONO, or —ONO₂ moieties. Pro-drugs can typically be prepared using well-known methods, such as those described by Burger's Medicinal Chemistry and Drug Discovery (1995) 172-178, 949-982 (Manfred E. Wolff ed., 5th ed.).

### Pharmaceutical Compositions and Methods of Administration:

The compounds disclosed herein, and their pharmaceutically acceptable salts, solvates, co-crystals and pro-drugs thereof, may be formulated as pharmaceutical compositions or “formulations”.

A typical formulation is prepared by mixing a compound of Formula I, or a pharmaceutically acceptable salt, solvate, co-crystal or pro-drug thereof, and a carrier, diluent or excipient. Suitable carriers, diluents and excipients are well known to those skilled in the art and include materials such as carbohydrates, waxes, water soluble and/or swellable polymers, hydrophilic or hydrophobic materials, gelatin, oils, solvents, water, and the like. The particular carrier, diluent or excipient used will depend upon the means and purpose for which the compound of Formula I is being formulated. Solvents are generally selected based on solvents recognized by persons skilled in the art as safe (e.g., GRAS-Generally Regarded as Safe) to be administered to a mammal. In general, safe solvents are non-toxic aqueous solvents such as water and other non-toxic solvents that are soluble or miscible in water. Suitable aqueous solvents include water, ethanol, propylene glycol, polyethylene glycols (e.g., PEG400, PEG300), etc., and mixtures thereof. The formulations may also include other types of excipients such as one or more buffers, stabilizing agents, antiadherents, surfactants, wetting agents, lubricating agents, emulsifiers, binders, suspending agents, disintegrants, fillers, sorbents, coatings (e.g., enteric or slow release) preservatives, antioxidants, opaquing agents, glidants, processing aids, colorants, sweeteners, perfuming agents, flavoring agents and other known additives to provide an elegant presentation of the drug (i.e., a compound of Formula I or pharmaceutical composition thereof) or aid in the manufacturing of the pharmaceutical product (i.e., medicament).

The formulations may be prepared using conventional dissolution and mixing procedures. For example, the bulk drug substance (i.e., compound of Formula I, a pharmaceutically acceptable salt, solvate, co-crystal or pro-drug thereof, or a stabilized form of the compound, such as a complex with a cyclodextrin derivative or other known complexation agent) is dissolved in a suitable solvent in the presence of one or more of the excipients described above. A compound having the desired degree of purity is optionally mixed with pharmaceutically acceptable diluents, carriers, excipients or stabilizers, in the form of a lyophilized formulation, milled powder, or an aqueous solution. Formulation may be conducted by mixing at ambient temperature at the appropriate pH, and at the desired degree of purity, with physiologically acceptable carriers. The pH of the formulation depends mainly on the particular use and the concentration of compound, but may range from about 3 to about 8.

The compound of Formula I or a pharmaceutically acceptable salt, solvate, co-crystal or pro-drug thereof is typically formulated into pharmaceutical dosage forms to provide an easily controllable dosage of the drug and to enable patient compliance with the prescribed regimen. Pharmaceutical formulations of compounds of Formula I, or a pharmaceutically acceptable salt, solvate, co-crystal or pro-drug thereof, may be prepared for various routes and types of administration. Various dosage forms may exist for the same compound, since different medical conditions may warrant different routes of administration. The amount of active ingredient that may be combined with the carrier material to produce a single dosage form will vary depending upon the subject treated and the particular mode of administration. For example, a time-release formulation intended for oral administration to humans may contain approximately 1 to 1000 mg of active material compounded with an appropriate and convenient amount of carrier material, which may vary from about 5 to about 95% of the total composition (weight:weight). The pharmaceutical composition can be prepared to provide easily measurable amounts for administration. For example, an aqueous solution intended for intravenous infusion may contain from about 3 to 500 μg of the active ingredient per milliliter of solution in order that infusion of a suitable volume at a rate of about 30 mL/hr can occur. As a general proposition, the initial pharmaceutically effective amount of the inhibitor administered will be in the range of about 0.01-100 mg/kg per dose, namely about 0.1 to 20 mg/kg of patient body weight per day, with the typical initial range of compound used being 0.3 to 15 mg/kg/day.

The term “therapeutically effective amount” as used herein means that amount of active compound or pharmaceutical agent that elicits the biological or medicinal response in a tissue, system, animal or human that is being sought by a researcher, veterinarian, medical doctor or other clinician. The therapeutically or pharmaceutically effective amount of the compound to be administered will be governed by such considerations, and is the minimum amount necessary to ameliorate, cure or treat the disease or disorder or one or more of its symptoms.

The pharmaceutical compositions of Formula I will be formulated, dosed, and administered in a fashion, i.e., the amounts, concentrations, schedules, courses, vehicles, and route(s) of administration, consistent with good medical practice. Factors for consideration in this context include the particular disorder being treated, the particular human or other mammal being treated, the clinical condition of the individual patient, the cause of the disorder, the site of delivery of the agent, the method of administration, the scheduling of administration, and other factors known to medical practitioners, such as the age, weight, and response of the individual patient.

The term “prophylactically effective amount” refers to an amount effective in preventing or substantially lessening the chances of acquiring a disease or disorder or in reducing the severity of the disease or disorder or one or more of its symptoms before it is acquired or before the symptoms develop. Roughly, prophylactic measures are divided between primary prophylaxis (to prevent the development of a disease) and secondary prophylaxis (whereby the disease has already developed and the patient is protected against worsening of its severity).

Acceptable diluents, carriers, excipients, and stabilizers are those that are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG). The active pharmaceutical ingredients may also be entrapped in microcapsules prepared, for example, by coacervation techniques or by interfacial polymerization, e.g., hydroxymethylcellulose or gelatin-microcapsules and poly-(methylmethacrylate) microcapsules, respectively, in colloidal drug delivery systems (for example, liposomes, albumin microspheres, microemulsions, nano-particles and nanocapsules) or in macroemulsions. Such techniques are disclosed in Remington's: The Science and Practice of Pharmacy, 21^{st} Edition, University of the Sciences in Philadelphia, Eds., 2005 (hereafter “Remington's”).

“Controlled drug delivery systems” supply the drug to the body in a manner precisely controlled to suit the drug and the conditions being treated. The primary aim is to achieve a therapeutic drug concentration at the site of action for the desired duration of time. The term “controlled release” is often used to refer to a variety of methods that modify release of drug from a dosage form. This term includes preparations labeled as “extended release”, “delayed release”, “modified release” or “sustained release”.

“Sustained-release preparations” are the most common applications of controlled release. Suitable examples of sustained-release preparations include semipermeable matrices of solid hydrophobic polymers containing the compound, wherein the matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and gamma-ethyl-L-glutamate, non-degradable ethylene-vinyl acetate, degradable lactic acid-glycolic acid copolymers, and poly-D-(−)-3-hydroxybutyric acid.

“Immediate-release preparations” may also be prepared. The objective of these formulations is to get the drug into the bloodstream and to the site of action as rapidly as possible. For instance, for rapid dissolution, most tablets are designed to undergo rapid disintegration to granules and subsequent disaggregation to fine particles. This provides a larger surface area exposed to the dissolution medium, resulting in a faster dissolution rate.

Implantable devices coated with a compound of this invention are another embodiment of the present invention. The compounds may also be coated on implantable medical devices, such as beads, or co-formulated with a polymer or other molecule, to provide a “drug depot”, thus permitting the drug to be released over a longer time period than administration of an aqueous solution of the drug. Suitable coatings and the general preparation of coated implantable devices are described in U.S. Pat. Nos. 6,099,562; 5,886,026; and 5,304,121. The coatings are typically biocompatible polymeric materials such as a hydrogel polymer, polymethyldisiloxane, polycaprolactone, polyethylene glycol, polylactic acid, ethylene vinyl acetate, and mixtures thereof. The coatings may optionally be further covered by a suitable topcoat of fluorosilicone, polysaccharides, polyethylene glycol, phospholipids or combinations thereof to impart controlled release characteristics in the composition.

The formulations include those suitable for the administration routes detailed herein. The formulations may conveniently be presented in unit dosage form and may be prepared by any of the methods well known in the art of pharmacy. Techniques and formulations generally are found in Remington's. Such methods include the step of bringing into association the active ingredient with the carrier which constitutes one or more accessory ingredients. In general, the formulations are prepared by uniformly and intimately bringing into association the active ingredient with liquid carriers or finely divided solid carriers or both, and then, if necessary, shaping the product.

The terms “administer”, “administering” or “administration” in reference to a compound, composition or formulation of the invention mean introducing the compound into the system of the animal in need of treatment. When a compound of the invention is provided in combination with one or more other active agents, “administration” and its variants are each understood to include concurrent and/or sequential introduction of the compound and the other active agents.

The compositions described herein may be administered systemically or locally, e.g.: orally (e.g., using capsules, powders, solutions, suspensions, tablets, sublingual tablets and the like), by inhalation (e.g., with an aerosol, gas, inhaler, nebulizer or the like), to the ear (e.g., using ear drops), topically (e.g., using creams, gels, liniments, lotions, ointments, pastes, transdermal patches, etc.), ophthalmically (e.g., with eye drops, ophthalmic gels, ophthalmic ointments), rectally (e.g., using enemas or suppositories), nasally, buccally, vaginally (e.g., using douches, intrauterine devices, vaginal suppositories, vaginal rings or tablets, etc.), via an implanted reservoir or the like, or parenterally depending on the severity and type of the disease being treated. The term “parenteral” as used herein includes, but is not limited to, subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally or intravenously.

The pharmaceutical compositions described herein may be orally administered in any orally acceptable dosage form including, but not limited to, capsules, tablets, aqueous suspensions or solutions. Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. Tablets may be uncoated or may be coated by known techniques including microencapsulation to mask an unpleasant taste or to delay disintegration and adsorption in the gastrointestinal tract and thereby provide a sustained action over a longer period. For example, a time-delay material such as glyceryl monostearate or glyceryl distearate alone or with a wax may be employed. A water soluble taste-masking material such as hydroxypropyl-methylcellulose or hydroxypropyl-cellulose may be employed.

Formulations of a compound of Formula I that are suitable for oral administration may be prepared as discrete units such as tablets, pills, troches, lozenges, aqueous or oil suspensions, dispersible powders or granules, emulsions, hard or soft capsules, e.g., gelatin capsules, syrups or elixirs. Formulations of a compound intended for oral use may be prepared according to any method known to the art for the manufacture of pharmaceutical compositions.

Compressed tablets may be prepared by compressing in a suitable machine the active ingredient in a free-flowing form such as a powder or granules, optionally mixed with a binder, lubricant, inert diluent, preservative, surface active or dispersing agent. Molded tablets may be made by molding in a suitable machine a mixture of the powdered active ingredient moistened with an inert liquid diluent.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water soluble carrier such as polyethyleneglycol or an oil medium, for example peanut oil, liquid paraffin, or olive oil.

The active compounds can also be in microencapsulated form with one or more excipients as noted above.

When aqueous suspensions are required for oral use, the active ingredient is combined with emulsifying and suspending agents. If desired, certain sweetening and/or flavoring agents may be added. Syrups and elixirs may be formulated with sweetening agents, for example glycerol, propylene glycol, sorbitol or sucrose. Such formulations may also contain a demulcent, a preservative, flavoring and coloring agents and antioxidant.

Sterile injectable forms of the compositions described herein (e.g., for parenteral administration) may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents which are commonly used in the formulation of pharmaceutically acceptable dosage forms including emulsions and suspensions. Other commonly used surfactants, such as Tweens, Spans and other emulsifying agents or bioavailability enhancers that are commonly used in the manufacture of pharmaceutically acceptable solid, liquid, or other dosage forms may also be used for the purposes of injectable formulations.

Oily suspensions may be formulated by suspending the compound of Formula I in a vegetable oil, for example arachis oil, olive oil, sesame oil or coconut oil, or in mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as butylated hydroxyanisol or alpha-tocopherol.

Aqueous suspensions of compounds of Formula I contain the active materials in admixture with excipients suitable for the manufacture of aqueous suspensions. Such excipients include a suspending agent, such as sodium carboxymethylcellulose, croscarmellose, povidone, methylcellulose, hydroxypropyl methylcelluose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethyleneoxycetanol), a condensation product of ethylene oxide with a partial ester derived from a fatty acid and a hexitol anhydride (e.g., polyoxyethylene sorbitan monooleate). The aqueous suspension may also contain one or more preservatives such as ethyl or n-propyl p-hydroxy-benzoate, one or more coloring agents, one or more flavoring agents and one or more sweetening agents, such as sucrose or saccharin.

The injectable formulations can be sterilized, for example, by filtration through a bacteria-retaining filter, or by incorporating sterilizing agents in the form of sterile solid compositions that can be dissolved or dispersed in sterile water or other sterile injectable medium prior to use.

In order to prolong the effect of a compound described herein, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsuled matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot-injectable formulations are also prepared by entrapping the compound in liposomes or microemulsions that are compatible with body tissues.

The injectable solutions or microemulsions may be introduced into a patient's bloodstream by local bolus injection. Alternatively, it may be advantageous to administer the solution or microemulsion in such a way as to maintain a constant circulating concentration of the instant compound. In order to maintain such a constant concentration, a continuous intravenous delivery device may be utilized. An example of such a device is the Deltec CADD-PLUS™ model 5400 intravenous pump.

Compositions for rectal or vaginal administration are preferably suppositories, which can be prepared by mixing the compounds described herein with suitable non-irritating excipients or carriers such as cocoa butter, beeswax, polyethylene glycol or a suppository wax that are solid at ambient temperature but liquid at body temperature and therefore melt in the rectum or vaginal cavity and release the active compound. Other formulations suitable for vaginal administration may be presented as pessaries, tampons, creams, gels, pastes, foams or sprays.

The pharmaceutical compositions described herein may also be administered topically, especially when the target of treatment includes areas or organs readily accessible by topical application, including diseases of the eye, the ear, the skin, or the lower intestinal tract. Suitable topical formulations are readily prepared for each of these areas or organs.

Dosage forms for topical or transdermal administration of a compound described herein include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, eardrops, and eye drops are also contemplated as being within the scope of this invention. Additionally, the present invention contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel. Topical application for the lower intestinal tract can be effected in a rectal suppository formulation (see above) or in a suitable enema formulation. Topically-transdermal patches may also be used.

For topical applications, the pharmaceutical compositions may be formulated in a suitable ointment containing the active component suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax and water. Alternatively, the pharmaceutical compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2 octyldodecanol, benzyl alcohol and water.

For ophthalmic use, the pharmaceutical compositions may be formulated as micronized suspensions in isotonic, pH-adjusted sterile saline, or, preferably, as solutions in isotonic, pH-adjusted sterile saline, either with or without a preservative such as benzylalkonium chloride. Alternatively, for ophthalmic uses, the pharmaceutical compositions may be formulated in an ointment such as petrolatum. For treatment of the eye or other external tissues, e.g., mouth and skin, the formulations may be applied as a topical ointment or cream containing the active ingredient(s) in an amount of, for example, 0.075 to 20% w/w. When formulated in an ointment, the active ingredients may be employed with either an oil-based, paraffinic or a water-miscible ointment base.

Alternatively, the active ingredients may be formulated in a cream with an oil-in-water cream base. If desired, the aqueous phase of the cream base may include a polyhydric alcohol, i.e., an alcohol having two or more hydroxyl groups such as propylene glycol, butane 1,3-diol, mannitol, sorbitol, glycerol and polyethylene glycol (including PEG 400) and mixtures thereof. The topical formulations may desirably include a compound which enhances absorption or penetration of the active ingredient through the skin or other affected areas. Examples of such dermal penetration enhancers include dimethyl sulfoxide and related analogs.

The oily phase of emulsions prepared using compounds of Formula I may be constituted from known ingredients in a known manner. While the phase may comprise merely an emulsifier (otherwise known as an emulgent), it desirably comprises a mixture of at least one emulsifier with a fat or an oil or with both a fat and an oil. A hydrophilic emulsifier may be included together with a lipophilic emulsifier which acts as a stabilizer. In some embodiments, the emulsifier includes both an oil and a fat. Together, the emulsifier(s) with or without stabilizer(s) make up the so-called emulsifying wax, and the wax together with the oil and fat make up the so-called emulsifying ointment base, which forms the oily dispersed phase of the cream formulations. Emulgents and emulsion stabilizers suitable for use in the formulation of compounds of Formula I include Tween™-60, Span™-80, cetostearyl alcohol, benzyl alcohol, myristyl alcohol, glyceryl mono-stearate and sodium lauryl sulfate.

The pharmaceutical compositions may also be administered by nasal aerosol or by inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents. Formulations suitable for intrapulmonary or nasal administration have a mean particle size, for example, in the range of 0.1 to 500 microns (including particles with a mean particle size in a range between 0.1 and 500 microns in micron increments such as 0.5, 1, 30, 35 microns, etc.), and are administered by rapid inhalation through the nasal passage or by inhalation through the mouth so as to reach the alveolar sacs.

For use, the pharmaceutical composition (or formulation) may be packaged in a variety of ways depending upon the method used for administering the drug. Generally, an article for distribution includes a container having deposited therein the pharmaceutical formulation in an appropriate form. Suitable containers are well-known to those skilled in the art and include materials such as bottles (plastic and glass), sachets, ampoules, plastic bags, metal cylinders, and the like. The container may also include a tamper-proof assemblage to prevent indiscreet access to the contents of the package. In addition, the container has deposited thereon a label that describes the contents of the container. The label may also include appropriate warnings.

The formulations may be packaged in unit-dose or multi-dose containers, for example sealed ampoules and vials, and may be stored in a freeze-dried (lyophilized) condition requiring only the addition of the sterile liquid carrier, for example water, for injection immediately prior to use. Extemporaneous injection solutions and suspensions are prepared from sterile powders, granules and tablets of the kind previously described. Preferred unit dosage formulations are those containing a daily dose or unit daily sub-dose, as recited herein above, or an appropriate fraction thereof of the active ingredient.

In another aspect, a compound of Formula I or a pharmaceutically acceptable salt, co-crystal, solvate or pro-drug thereof may be formulated in a veterinary composition comprising a veterinary carrier. Veterinary carriers are materials useful for the purpose of administering the composition and may be solid, liquid or gaseous materials that are otherwise inert or acceptable in the veterinary art and are compatible with the active ingredient. These veterinary compositions may be administered parenterally, orally or by any other desired route.

### Therapeutic Methods:

The terms, “disease”, “disorder”, and “condition” may be used interchangeably here to refer to a condition where an increase in the concentration of an endogenous cannabinoid (eCB) might be beneficial or a condition that can be treated by a FAAH inhibitor.

As used herein, the terms “subject” and “patient” are used interchangeably. The terms “subject” and “patient” refer to an animal (e.g., a bird such as a chicken, quail or turkey, or a mammal), preferably a “mammal” including a non-primate (e.g., a cow, pig, horse, sheep, rabbit, guinea pig, rat, cat, dog, and mouse) and a primate (e.g., a monkey, chimpanzee and a human), and more preferably a human. In one embodiment, the subject is a non-human animal such as a farm animal (e.g., a horse, cow, pig or sheep), or a pet (e.g., a dog, cat, guinea pig or rabbit). In a preferred embodiment, the subject is a “human”.

The term “biological sample”, as used herein, includes, without limitation, in vivo or ex vivo cell cultures or extracts thereof; biopsied material obtained from a mammal or extracts thereof; blood, saliva, urine, feces, semen, tears, lymphatic fluid, ocular fluid, vitreous humor or other body fluids or extracts thereof.

“Treat”, “treating” or “treatment” with regard to a disorder or disease refers to alleviating or abrogating the cause and/or the effects of the disorder or disease. As used herein, the terms “treat”, “treatment” and “treating” refer to the reduction or amelioration of the progression, severity and/or duration of a condition wherein an increase in the concentration of eCB might be beneficial or that can be treated with a FAAH inhibitor, or the amelioration of one or more symptoms (preferably, one or more discernible symptoms) of said condition, resulting from the administration of one or more therapies (e.g., one or more therapeutic agents such as a compound or composition of the invention). In specific embodiments, the terms “treat”, “treatment” and “treating” refer to the amelioration of at least one measurable physical parameter of condition wherein an increase in the concentration of eCB might be beneficial or a condition that can be treated with a FAAH inhibitor. In other embodiments the terms “treat”, “treatment” and “treating” refer to the inhibition of the progression of said condition, either physically by, e.g., stabilization of a discernible symptom, physiologically by, e.g., stabilization of a physical parameter, or both.

As used herein, the terms “prevent”, “preventing” and “prevention” with regard to a disorder or disease refer to averting the cause and/or effects of a disease or disorder prior to the disease or disorder manifesting itself. The terms “prophylaxis” or “prophylactic use”, as used herein, refer to any medical or public health procedure whose purpose is to prevent, rather than treat or cure a disease. As used herein, the terms “prevent”, “prevention” and “preventing” refer to the reduction in the risk of acquiring or developing a given condition, or the reduction or inhibition of the recurrence or said condition in a subject who is not ill, but who has been or may be near a person with the disease.

The term “chemotherapy” refers to the use of medications, e.g., small molecule drugs (rather than e.g., “vaccines”) for treating a disorder or disease.

The term “chemoprophylaxis” refers to the use of medications, e.g., small molecule drugs (rather than e.g., “vaccines”) for the prevention of a disorder or disease.

In one embodiment, the methods of the invention are a preventative or “pre-emptive” measure to a patient, preferably a human, having a predisposition to developing a condition or symptom that can be improved by an increase in the concentration of an eCB or treated with a FAAH inhibitor.

Also described are methods for treating or preventing various disorders with a composition that includes any of the various embodiments of the compound of Formula I. Among the disorders or symptoms that can be treated or prevented are: pain (e.g., acute pain, chronic pain, neurogenic pain, dental pain, menstrual pain, dysmenorrheal pain, visceral pain, neuropathic pain, post operative pain, headache, migraines, allodynia, hyperalgesia, post operative pain (e.g., associated with orthopedic surgery, gynecological surgery, abdominal surgery, incisions, oral surgery), back pain, pain caused by inflammation (e.g., arthritis, osteoarthritis, spondylitis, rheumatoid arthritis, Crohn's disease and irritable bowel syndrome) and pain associated with injury, burns or trauma), anxiety, depression, an eating disorder (e.g., anorexia and bulimia), obesity (e.g., by appetite suppression), elevated intraocular pressure (e.g., glaucoma), a cardiovascular disorder (e.g., hypertension), an inflammatory disorder (e.g., allergy (e.g., food allergy-, respiratory inflammation, inflammation of the skin and gastrointestinal inflammation), asthma, Crohn's disease), emesis (e.g., as a side effect of chemotherapy), some cancers, excitotoxic insult (e.g., in cerebral ischemia, seizure and edema due to traumatic brain injury), asphyxia, addictive behaviors, sleep disorders, epilepsy, epileptiform-induced damage, progressive CNS diseases (e.g., Parkinson's, motor neuron disorders, ALS (amyotropic lateral sclerosis), Huntington's and motor dysfunction), gastrointestinal disorders (e.g., attenuation of cholera induced fluid accumulation, nausea, vomiting, gastric ulcers, diarrhea, paralytic ileus, IBS, and gastro-esophageal reflux conditions) and autoimmune disorders (e.g., Multiple Sclerosis).

The compounds and pharmaceutical compositions described herein can be used alone or in combination therapy for the treatment or prevention of pain. The pain can be chronic pain, acute pain, perioperative pain (e.g., associated with surgery), postoperative pain, visceral pain, inflammatory pain, cancer pain, headache pain, pain associated with cough, neuropathic pain, deafferentation pain, chronic nociceptive pain, dental pain (such as odontalgia), bone pain, joint pain (e.g., osteoarthritis or rheumatoid arthritis), myofascial pain (e.g., muscular injury, fibromyalgia), labor pain, pain associated with injuries, trauma, allergies, dermatitis, immunodeficiency, Hodgkin's disease, Myasthenia gravis, nephrotic syndrome, scleroderma, or thyroiditis, central and peripheral pathway mediated pain, or pain associated with or the result of injury or age.

Neuropathic pain is initiated or caused by a primary lesion or dysfunction in the peripheral or central nervous systems. It can occur in the peripheral nerves, dorsal roots, spinal cord and certain regions of the brain. It can also result from a peripheral nerve disorder such as neuroma, nerve compression, nerve crush, nerve stretch or incomplete nerve transsection. It can be associated with neuronal lesions, such as those induced by diabetes, HIV, herpes infection, nutritional deficiencies or a stroke. Chronic neuropathic pain can result from injury and/or inflammation such as chronic lower back pain. Acute neuropathic pain includes, for example, traumatic pain (e.g., bone fracture pain, sprains, strains and soft tissue damage), muscle pain, burn pain, and sun burn pain. Neuropathic pain can also be associated with, for example, nerve injury, head trauma, hyperalgesia, allodynia, dysesthesias, sciatica, amputation (e.g., phantom limb syndrome, stump pain), fibromyalgia, chemotherapeutic neuropathy, cancer pain (e.g., tumors of the brainstem, thalamus or cortex), AIDS-related neuropathy, painful traumatic mononeuropathy, painful polyneuropathy, multiple sclerosis, root avulsions, post-thoracotomy syndrome. It can be the result of a central nervous system injury (such as pain in stroke or spinal cord injury patients). Neuropathic pain also includes lower back pain, toxin induced pain, neurogenic pain, thalamic pain syndrome, repetitive motion pain (e.g., carpal tunnel syndrome) or pain induced by post mastectomy syndrome, by surgery or by radiation. Neuralgia is a type of neuropathic pain that is thought to be linked to four possible mechanisms: ion gate malfunctions; a nerve becoming mechanically sensitive and creating an ectopic signal; cross signals between large and small fibers; and malfunction due to damage in the central processor. Under the general heading of neuralgia are trigeminal neuralgia (TN), atypical trigeminal neuralgia (ATN), and post-herpetic neuralgia (caused by shingles or herpes). Neuralgia is also involved in disorders such as sciatica and brachial plexopathy with neuropathia. Neuralgias that do not involve the trigeminal nerve are occipital neuralgia and glossopharyngeal neuralgia. Neuropathic pain also includes referred pain.

Visceral pain includes, for example, pain associated with pancreatitis, peptic ulcers, interstitial cystitis, renal colic, angina, dysmenorrhea, menstrual cramps, menstruation, irritable bowel syndrome (IBS), myocardial ischemia, and non-ulcer dyspepsia. Visceral pain also includes gynecological pain, urinary bladder pain, kidney pain, non-cardiac chest pain, and chronic pelvic pain.

Inflammatory pain includes both inflammatory pain that is a significant component of a disorder or disease and that that is considered a minor component or symptom. For example, inflammatory pain induced by or associated with disorders such as osteoarthritis, rheumatic fever, rheumatoid arthritis, rheumatic disease, tendonitis, juvenile arthritis, spondylitis, gouty arthritis, psoriatic arthritis, interstitial cystitis, peripheral neuritis, mucositis, fibromyalgia, pancreatitis, enteritis, diverticulitis, cellulites, bone fractures, post-operative ileus, Crohn's Disease, ulcerative colitis, cholecystitis, teno-synovitis, gout, vulvodynia, fibromyalgia, sprains and strains, systemic lupus erythematosus, myositis, bronchitis and influenza and other viral infections such as the common cold. Inflammatory pain also includes sympathetically maintained pain, pain due to venomous and non-venomous snake bite, spider bite or insect sting, sports injury pain, sprain pain, joint pain, myofascial pain (muscular injury, fibromyalgia), muscoskeletal pain, and pain due to inflammatory bowel diseases. Among the inflammatory pain disorders that can be treated are included some autoimmune disorders or diseases.

Cancer pain can be induced by or associated with tumors such as lymphatic leukemia, Hodgkin's disease, malignant lymphoma, osteosarcoma, bone cancer, lymphogranulomatoses, lymphosarcoma, solid malignant tumors, and extensive metastases. Chemotherapy pain is a side effect of chemotherapy treatments.

Headache pain includes cluster headache, migraines with and without aura, tension type headache, headaches caused by injury or infection, hangovers, and headaches with unknown origins.

The compounds and pharmaceutical compositions described herein can be used alone or in combination therapy for the treatment or prevention of inflammatory disorders, including, for example, chronic and acute inflammatory disorders. Examples of disorders with inflammatory components include asthma, atopic allergy, allergy, atherosclerosis, bronchial asthma, eczema, glomerulonephritis, graft vs. host disease, hemolytic anemia, osteoarthritis, sepsis, septic shock (e.g., as antihypovolemic and/or antihypotensive agents), systemic lupus erythematosus, stroke, transplantation of tissue and organs, vasculitis, diabetic retinopathy and ventilator induced lung injury. The compounds and pharmaceutical compositions described herein can also be used alone or in combination therapy for the treatment or prevention of disease states or indications that are accompanied by inflammatory processes such as:

(1) Lung diseases: e.g., asthma, bronchitis, allergic rhinitis, emphysema, adult respiratory distress syndrome (ARDS), pigeon fancier's disease, farmer's lung, chronic obstructive pulmonary disease (COPD), asthma including allergic asthma (atopic or non-atopic) as well as exercise-induced bronchoconstriction, occupational asthma, viral or bacterial exacerbation of asthma, other non-allergic asthmas and “wheezy-infant syndrome”, pneumoconiosis, including aluminosis, anthracosis, asbestosis, chalicosis, ptilosis, siderosis, silicosis, tabacosis and byssinosis;

(2) Rheumatic diseases or autoimmune diseases or musculoskeletal diseases: e.g., all forms of rheumatic diseases, especially rheumatoid arthritis, acute rheumatic fever, and polymyalgia rheumatica; reactive arthritis; rheumatic soft tissue diseases; inflammatory soft tissue diseases of other genesis; arthritic symptoms in degenerative joint diseases (arthroses); tendinitis, bursitis, osteoarthritis, traumatic arthritis, gout (metabolic arthritis); collagenoses of any genesis, e.g., systemic lupus erythematosus, scleroderma, polymyositis, dermatomyositis, Sjogren syndrome, Still disease, Felty syndrome; and osteoporosis and other bone resorption diseases;

(3) Allergic diseases including all forms of allergic reactions, e.g., allergic rhinitis, allergic conjunctivitis, infectious parasitic, angioneurotic edema, hay fever, insect bites, allergic reactions to drugs, blood derivatives, contrast agents, etc., anaphylactic shock (anaphylaxis), urticaria, angioneurotic edema, delayed or immediate hypersensitivity, and contact dermatitis;

(4) Vascular diseases: e.g., panarteritis nodosa, polyarteritis nodosa, periarteritis nodosa, arteritis temporalis, Wegner granulomatosis, giant cell arthritis, atherosclerosis, reperfusion injury and erythema nodosum, myocardial ischemia, thrombosis.

(5) Dermatological diseases: e.g., dermatitis, psoriasis, sunburn, burns, and eczema;

(6) Renal, urinary and pancreatic diseases: e.g., nephrotic syndrome and all types of nephritis (such as glomerulonephritis); pancreatitis; bladder hyperrelexia following bladder inflammation; other renal diseases that can be treated by the compounds and compositions herein described include urinary incontinence or vesicle inflammation, uresesthesia urgency, overactive bladder, urinary frequency, interstitial cystitis or chronic prostatitis;

(7) Hepatic diseases: e.g., acute liver cell disintegration; acute hepatitis of various genesis (such as viral, toxic, drug-induced) and chronically aggressive and/or chronically intermittent hepatitis, liver fibrosis associated with liver injury or disease, including fibrosis caused or exacerbated by alcoholic liver cirrhosis, chronic viral hepatitis, non alcoholic steatohepatitis and primary liver cancer;

(8) Gastrointestinal diseases: e.g., ulcers, inflammatory bowel diseases, regional enteritis (Crohn's disease), ulcerative colitis, gastritis, aphthous ulcer, celiac disease, regional ileitis, ileus, esophagitis, NSAID-induced ulcer, non-ulcerative dyspepsia and gastroesophageal reflux disease;

(9) Neurodegenerative diseases: e.g., treatment/reduction of neurodegeneration following stroke, cardiac arrest, pulmonary bypass, traumatic brain injury, edema, spinal cord injury, cerebral ischemia, seizures or the like;

(10) Eye diseases: e.g., allergic keratitis, uveitis, or iritis, conjunctivitis, blepharitis, neuritis nervi optici, choroiditis, glaucoma and sympathetic ophthalmia;

(11) Diseases of the ear, nose, and throat (ENT) area: e.g., tinnitus, allergic rhinitis or hay fever, gingivitis, otitis externa, caused by contact eczema, infection, etc., and otitis media;

(12) Progressive central nervous system or neurological diseases: e.g., brain edema, particularly tumor-related brain edema, multiple sclerosis, acute encephalomyelitis, meningitis, acute spinal cord injury, trauma; cognitive disorders such as dementia, particularly degenerative dementia (including senile dementia, Alzheimer's disease, Parkinson's disease and Creutzfeldt-Jacob disease, Huntington's chorea, Pick's disease, amyotrophic lateral sclerosis (ALS)), vascular dementia (including multi-infarct dementia and dementia associated with intracranial space occupying lesions, infections and related conditions such as HIV infection); Guillain-Barre syndrome, myasthenia gravis, stroke, and various forms of seizures (such as nodding spasms);

(13) Blood diseases: e.g., acquired hemolytic anemia, aplastic anemia, and idiopathic thrombocytopenia;

(14) Tumor diseases: e.g., acute lymphatic leukemia, Hodgkin's disease, malignant lymphoma, lymphogranulomatoses, lymphosarcoma, solid malignant tumors, colorectal polyps, and extensive metastases; other proliferative disorders such as diabetic retinopathy and tumor angiogenesis (e.g., wet macular degeneration).

(15) Endocrine diseases: e.g., endocrine opthalmopathy, endocrine orbitopathia, thyrotoxic crisis, Thyroiditis de Quervain, Hashimoto thyroiditis, Morbus Basedow, granulomatous thyroiditis, struma lymphomatosa, Graves disease, type I diabetes (such as insulin-dependent diabetes); organ and tissue transplantations and graft vs. host diseases;

(16) Severe states of shock: e.g., septic shock, anaphylactic shock, and systemic inflammatory response syndrome (SIRS);

(17) Viral or bacterial parasitic infectious disease: for example AIDS and meningitis; and

(18) various other disease-states or conditions including, restenosis following percutaneous transluminal coronary angioplasty, acute and chronic pain, atherosclerosis, reperfusion injury, congestive heart failure, myocardial infarction, thermal injury, multiple organ injury secondary to trauma, necrotizing enterocolitis and syndromes associated with hemodialysis, leukopheresis, granulocyte transfusion, sarcoidosis, gingivitis, pyrexia; edema resulting from trauma associated with burns, sprains or fracture, cerebral edema and angioedema, and diabetes (such as diabetic vasculopathy, diabetic neuropathy, diabetic retinopathy, post capillary resistance and diabetic symptoms associated with insulitis (e.g., hyperglycemia, diuresis, proteinuria and increased nitrite and kallikrein urinary excretion)).

The compounds and compositions herein described can be used alone or in combination therapy for the treatment of gastrointestinal (GI) diseases or disorders: e.g., functional gastrointestinal disorders, ulcers, inflammatory bowel diseases (IBD), regional enteritis (Crohn's disease), ulcerative colitis, diarrhea, gastritis, aphthous ulcer, celiac disease, regional ileitis, ileus, functional dyspepsia, diverticulitis, gastrointestinal bleeding, irritable bowel syndrome (IBS), non-ulcerative dyspepsia and gastroesophageal reflux disease.

The compounds and pharmaceutical compositions described herein can be used alone or in combination therapy for the treatment or prevention of pruritus (itch). For example, pruritus originated in the skin (dermal pruritus), neuropathic pruritus, neurogenic or psychogenic pruritus would all be included. Pruritus (itching) can be a symptom of primary skin diseases or of systemic disease. Skin diseases notorious for causing intense pruritus include scabies, pediculosis, insect bites, urticaria, atopic and contact dermatitis, lichen planus, miliaria, and dermatitis herpetiformis. In other cases pruritus is prominent without any identifiable skin lesions: e.g., dry skin (especially in elderly people), systemic disease, and use of certain drugs can generate pruritus. Systemic diseases that cause generalized pruritus include cholestatic diseases, uremia, polycythemia vera, and hematologic malignancies. Pruritus may also occur during the later months of pregnancy. Barbiturates, salicylates, morphine and cocaine can cause pruritus. Less well-defined causes of pruritus include hyper- and hypothyroidism, diabetes, iron deficiency, and internal cancers of many types.

The compounds and pharmaceutical compositions described herein can be used alone or in combination therapy for the treatment or prevention of substance abuse related syndromes, disorders or diseases include, including, for example, drug abuse and drug withdrawal. Abused substances can include alcohol, amphetamines, amphetamine-like substances, caffeine, cannabis, cocaine, hallucinogens, inhalants, opioids, nicotine (and/or tobacco products), heroin, barbiturates, phencyclidine (or phencyclidine-like compounds), sedative-hypnotics, benzodiazepines, or combinations of any of the foregoing. The compounds and pharmaceutical compositions can also be used to treat withdrawal symptoms and substance-induced anxiety or mood disorder. In addition, they can be used to reduce tobacco craving; treat nicotine dependency, addiction, or withdrawal; or aid in the cessation or lessening of tobacco in a subject in need thereof.

The compounds and pharmaceutical compositions described herein can be used alone or in combination therapy for the treatment or prevention of psychiatric disorders, such as depressions (including major depressive disorder, bipolar depression, unipolar depression, single or recurrent major depressive episodes (e.g., with or without psychotic features, catatonic features, and/or melancholic features), postpartum onset, seasonal affective disorder, dysthymic disorders (e.g., with early or late onset and with or without atypical features), neurotic depression and social phobia, depression accompanying dementia, anxiety, psychosis, social affective disorders, and/or cognitive disorders); manic-depressive psychoses; bipolar disorders; extreme psychotic states (such as mania, schizophrenia, and excessive mood swings where behavioral stabilization is desired), \; Post-traumatic stress disorder; panic disorder; obsessive compulsive disorder. The compounds and pharmaceutical compositions described herein can also be used alone or in combination therapy for the treatment or prevention of attention disorders such as ADHD (attention deficit hyperactivity disorders), autism, anxiety states, generalized anxiety, agoraphobia, as well as those behavioral states characterized by social withdrawal. They can also be used for the treatment of psychiatric tremors (e.g., dyskinesias, dystonia, spasticity).

The compounds and pharmaceutical compositions described herein can be used alone or in combination therapy for the treatment or prevention of an autoimmune disease or disorder or at least one symptom associated with said disease or disorder, including, for example, alopecia greata (also known as systemic sclerosis (SS)), amyloses, amyotrophic lateral sclerosis, ankylosing spondylarthritis, ankylosing spondylitis, antiphospholipid syndrome, autoimmune Addison's disease, autoimmune hemolytic anemia, autoimmune hepatitis, autoimmune inner ear disease (AIED), autoimmune lymphoproliferative syndrome (ALPS), autoimmune thrombocytopenic purpura (ATP), Behcet's disease, cardiomyopathy, celiac sprue-dermatitis hepetiformis; chronic fatigue immune dysfunction syndrome (CFIDS), chronic inflammatory demyelinating polyneuropathy (CIPD), cicatricial pemphigold, cold agglutinin disease, connective tissue diseases, crest syndrome, Crohn's disease, Degos' disease, dermatomyositis-juvenile, discoid lupus, essential mixed cryoglobulinemia, fibromyalgia-fibromyositis, graft vs. host disease, transplantation rejection, Graves' disease, Guillain-Barre syndrome, Hashimoto's thyroiditis, idiopathic pulmonary fibrosis, idiopathic thrombocytopenia purpura (ITP), IgA nephropathy, insulin-dependent diabetes mellitus, juvenile chronic arthritis (Still's disease), juvenile rheumatoid arthritis, lupus erythematosus, Meniere's disease, multiple sclerosis, myasthenia gravis, pernicious anemia, polyarteritis nodosa, polychondritis, polyglandular syndromes, polymyalgia rheumatica, polymyositis and dermatomyositis, primary agammaglobulinemia, primary biliary cirrhosis, psoriasis, psoriatic arthritis, Raynaud's phenomena, rectional arthritis, Reiter's syndrome, rheumatic fever, rheumatoid arthritis, sarcoidosis, scleroderma (progressive systemic sclerosis (PSS), Sjogren's syndrome, stiff-man syndrome, systemic lupus erythematosus, Takayasu arteritis, temporal arteritis/giant cell arteritis, ulcerative colitis, undifferentiated spondylarthritis, uveitis, vitiligo, and Wegener's granulomatosis.

The compounds and pharmaceutical compositions described herein can be used alone or in combination therapy for the treatment or prevention of neurological or neurodegenerative disorders. Examples of neurodegenerative diseases include dementia, particularly degenerative dementia (including senile dementia, Alzheimer's disease, Pick's disease, Huntingdon's chorea, Parkinson's disease, prion disease and Creutzfeldt-Jakob disease, motor neuron disease); vascular dementia (including multi-infarct dementia); as well as dementia associated with intracranial space occupying lesions; trauma; infections and related conditions (including HIV infection); dementia in Parkinson's disease; metabolism; toxins; anoxia and vitamin deficiency; and mild cognitive impairment associated with aging, particularly Age-Associated Memory Impairment. Examples of neurological disorders include amyotrophic lateral sclerosis (ALS), multiple sclerosis, epilepsy, ischemia, traumatic head or brain injury, brain inflammation, eye injury, stroke and neuroinflammation. The compounds and compositions here described can also be used for the treatment/reduction of neurodegeneration or decreased brain activity associated with stroke, cardiac arrest, pulmonary bypass, traumatic brain injury, hypoxia, hypoglycemia, gas poisoning, drug intoxication, diabetes mellitus, edema, spinal cord injury, cerebral ischemia, cerebral infarction, cerebral hemorrhage, subarachnoid hemorrhage, seizures or the like.

The compounds and pharmaceutical compositions described herein can be used alone or in combination therapy for the treatment or prevention of ocular disorders including, for example, glaucoma (such as normal tension glaucoma), glaucoma-associated intraocular pressure retinitis, retinopathies, uveitis, and acute injury to the eye tissue (e.g., conjunctivitis). Ocular disorders also include neurodegenerative disease conditions of the retina and the optic nerve, for example, in patients presenting risk factors for glaucoma, such as high intraocular pressure, family history of glaucoma, glaucoma in the contralateral eye and high myopia.

The compounds and compositions described herein can also be used, alone or in combination therapy, to treat or prevent appetite related disorders such as emesis, vomiting and nausea, food behavioral problems or feeding disorders (e.g., anorexias, cachexias, wasting conditions and bulimia) and obesity or obesity-related disorders (e.g., diabetes type II, hyperlipidemia).

Certain gynecological disorders can be treated by inhibition of uterus contraction caused by hormones and prostanoid-induced muscle contraction using compounds or compositions described herein, for example, premature labor, menstrual cramps, menstrual irregularity, dysmenorrhea.

Some sleep disorders can be treated with compounds or compositions described herein, for example, insomnia, night terrors, bruxism, somnambulism, narcolepsy, circadian rhythm adjustment disorders, and the like. Also contemplated are sleep disorders associated with neurological or mental disorders or with pain.

Cardiovascular diseases that can be treated with the compounds and compositions described herein include myocardial ischemia, thrombosis, hypertension or cardiac arrhythmias.

Compounds and compositions of the invention are also useful for veterinary treatment of companion animals, exotic animals and farm animals, including, without limitation, dogs, cats, mice, rats, hamsters, gerbils, guinea pigs, rabbits, horses, pigs and cattle.

In another embodiment, the invention provides a method of inhibiting FAAH in a biological sample, comprising contacting said biological sample with a compound or composition of the invention. Use of a FAAH inhibitor in a biological sample is useful for a variety of purposes known to one of skill in the art. Examples of such purposes include, without limitation, biological assays and biological specimen storage.

### Combination Therapies:

The compounds and pharmaceutical compositions described herein can be used in combination therapy with one or more additional therapeutic agents. For combination treatment with more than one active agent, where the active agents are in separate dosage formulations, the active agents may be administered separately or in conjunction. In addition, the administration of one element may be prior to, concurrent to, or subsequent to the administration of the other agent.

When co-administered with other agents, e.g., when co-administered with another pain medication, an “effective amount” of the second agent will depend on the type of drug used. Suitable dosages are known for approved agents and can be adjusted by the skilled artisan according to the condition of the subject, the type of condition(s) being treated and the amount of a compound described herein being used. In cases where no amount is expressly noted, an effective amount should be assumed. For example, compounds described herein can be administered to a subject in a dosage range from between about 0.001 to about 100 mg/kg body weight/day, from about 0.001 to about 50 mg/kg body weight/day, from about 0.001 to about 30 mg/kg body weight/day, from about 0.001 to about 10 mg/kg body weight/day.

When combination therapy is employed, an effective amount can be achieved using a first amount of a compound of Formula I or a pharmaceutically acceptable salt, solvate (e.g., hydrate), co-crystal or pro-drug thereof and a second amount of an additional suitable therapeutic agent (e.g., an agent to treat pain).

In one embodiment of this invention, the compound of Formula I and the additional therapeutic agent, are each administered in an effective amount (i.e., each in an amount which would be therapeutically effective if administered alone). In another embodiment, the compound of Structural Formula I and the additional therapeutic agent, are each administered in an amount which alone does not provide a therapeutic effect (a sub-therapeutic dose). In yet another embodiment, the compound of Structural Formula I can be administered in an effective amount, while the additional therapeutic agent is administered in a sub-therapeutic dose. In still another embodiment, the compound of Structural Formula I can be administered in a sub-therapeutic dose, while the additional therapeutic agent, for example, a suitable cancer-therapeutic agent is administered in an effective amount.

As used herein, the terms “in combination” or “co-administration” can be used interchangeably to refer to the use of more than one therapy (e.g., one or more prophylactic and/or therapeutic agents). The use of the terms does not restrict the order in which therapies (e.g., prophylactic and/or therapeutic agents) are administered to a subject.

Co-administration encompasses administration of the first and second amounts of the compounds in an essentially simultaneous manner, such as in a single pharmaceutical composition, for example, capsule or tablet having a fixed ratio of first and second amounts, or in multiple, separate capsules or tablets for each. In addition, such coadministration also encompasses use of each compound in a sequential manner in either order. When co-administration involves the separate administration of a first amount of a compound of Formula I and a second amount of an additional therapeutic agent, the compounds are administered sufficiently close in time to have the desired therapeutic effect. For example, the period of time between each administration that can result in the desired therapeutic effect can range from minutes to hours and can be determined taking into account the properties of each compound such as potency, solubility, bioavailability, plasma half-life and kinetic profile. For example, a compound of Formula I and the second therapeutic agent can be administered in any order within about 24 hours of each other, within about 16 hours of each other, within about 8 hours of each other, within about 4 hours of each other, within about 1 hour of each other or within about 30 minutes of each other.

More specifically, a first therapy (e.g., a prophylactic or therapeutic agent such as a compound described herein) can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks prior to), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks subsequent to) the administration of a second therapy (e.g., a prophylactic or therapeutic agent such as an anti-cancer agent) to a subject.

Additional therapeutic agents that can be combined with compounds described herein include, without limitation:

FAAH inhibitors: e.g., OL-135, LY2183240, URB-597, CAY-10402, PF-750, BMS-469908, SSR-411298, TK-25, PF-04457845, PF-3845, SA-47, JNJ-245, JNJ-28833155 and JNJ-1661010;

painkillers such as acetaminophen or paracetamol;

non-steroidal anti-inflammatory drugs (NSAIDs) such as propionic acid derivatives (alminoprofen, benoxaprofen, bucloxic acid, carprofen, fenhufen, fenoprofen, flurbiprofen, ibuprofen, indoprofen, ketoprofen, miroprofen, naproxen, oxaprozin, pirprofen, pranoprofen, suprofen, tiaprofenic acid, and tioxaprofen), acetic acid derivatives (indomethacin, acemetacin, alclofenac, clidanac, diclofenac, fenclofenac, fenclozic acid, fentiazac, furofenac, ibufenac, isoxepac, oxpinac, sulindac, tiopinac, tolmetin, zidometacin, and zomepirac), fenamic acid derivatives (meclofenamic acid, mefe-namic acid, and tolfenamic acid), biphenyl-carboxylic acid derivatives, oxicams (isoxicam, meloxicam, piroxicam, sudoxicam and tenoxican), salicylates (acetyl salicylic acid, sulfasalazine) and the pyrazolones (apazone, bezpiperylon, feprazone, mofebutazone, oxyphenbutazone, phenylbutazone), and COX-2 inhibitors, such as the coxibs (celecoxib, deracoxib, valdecoxib, rofecoxib, parecoxib and etoricoxib);

other pain relieving agents such as gabapentin, topical capsaicin, tanezumab, esreboxetine;

opiate receptor agonists such as morphine, propoxyphene (Darvon), tramadol, buprenorphin;

cannabinoid receptor agonists such as Dronabinol, 6,9-THC, CP-55940, WIN-55212-2, HU-210;

anti-infective agents;

sodium channel blockers such as carbamazepine, mexiletine, lamotrigine, pregabalin, tectin, NW-1029, CGX-1002;

N-type calcium channel blockers such as Ziconotide, NMED-160, SPI-860; serotonergic and noradrenergic modulators such as SR-57746, paroxetine, duloxetine, clonidine, amitriptyline, citalopram;

local anesthetics such as ambroxol, lidocaine;

VR1 agonists and antagonists such as NGX-4010, WL-1002, ALGRX-4975, WL-10001, AMG-517;

agents used for migraines, such as sumatriptan, zolmitriptan, naratriptan, eletriptan, rauwolscine, yohimbine, metoclopramide;

topical agents used in the treatment of localized pruritus: e.g., camphor/menthol lotions or creams containing 0.125 to 0.25% menthol, doxepin (e.g., Sinequan, Zonalon), phenol (e.g., Cepastat, Chloraseptic gargle, Ulcerease), 0.5 to 2%, pramoxine (e.g., Anusol ointment, Proctofoam-NS, Tronolane Cream, Tucks Hemorrhoidal), eutectic mixture of local anesthetics (EMLA), and corticosteroids;

anti-inflammatory and/or immunosuppressive agents such as methotrexate, cyclosporin A (including, for example, cyclosporin microemulsion), tacrolimus, corticosteroids, statins, interferon beta, Remicade (Infliximab), Enbrel (Etanercept) and Humira (Adalimumab);

agents designed to treat tobacco abuse: e.g., nicotine receptor partial agonists, bupropion hypochloride (also known under the tradename Zyban™) and nicotine replacement therapies;

ADD/ADHD agents: e.g., Ritalin™ (methylphenidate hydrochloride), Strattera™ (atomoxetine hydrochloride), Concerta™ (methylphenidate hydrochloride) and Adderall™ (amphetamine aspartate; amphetamine sulfate; dextroamphetamine saccharate; and dextroamphetamine sulfate);

agents to treat alcoholism, such as opioid antagonists (e.g., naltrexone (also known under the tradename ReVia M) and nalmefene), disulfuram (also known under the tradename Antabuse™), and acamprosate (also known under the tradename Campral™));

agents for reducing alcohol withdrawal symptoms such as benzodiazepines, beta-blockers, clonidine, carbamazepine, pregabalin, and gabapentin (Neurontin™);

antihypertensive agents: e.g., ACE inhibitors and Angiotensin II Receptor blockers such as benazepril, captopril, enalapril, fosinopril, lisinopril, candesartan, eprosartan, Irbesartan, losartan, olmesartan, telmisartan, valsartan, Renin inhibitors such as aliskiren, vasodilators such as minoxidil;

agents used to treat glaucoma: e.g., direct-acting Miotics (cholinergic agonists), indirect acting Miotics (cholinesterase inhibitors), Carbonic anhydrase inhibitors (e.g., Acetazolamide, Methazolamide, Brinzolamide, Dorzolamide), Selective adrenergic agonists (e.g., Apraclonidine, Brimonidine), Beta-blockers (Timolol, Betaxolol, Carteolol, Levobetaxolol, Levobunolol, Metipranolol), Osmotic diuretics (e.g., Glycerin, Mannitol);

antidepressants: e.g., SSRIs (e.g., fluoxetine, citalopram, femoxetine, fluvoxamine, paroxetine, indalpine, sertraline, zimeldine), tricyclic antidepressants (e.g., imipramine, amitriptiline, chlomipramine and nortriptiline), dopaminergic antidepressants (e.g., bupropion and amineptine), SNRIs (e.g., venlafaxine and reboxetine);

cognitive improvement agents: e.g., donepezil hydrochloride (Aricept™) and other acetylcholinesterase inhibitors;

anti-emetic agents: e.g., 5HT3 antagonists such as ondansetron, granisetron, metoclopramide;

neuroprotective agents: e.g., memantine, L-dopa, bromocriptine, pergolide, talipexol, pramipexol, cabergoline, neuroprotective agents currently under investigation including anti-apoptotic drugs (CEP 1347 and CTCT346), lazaroids, bioenergetics, antiglutamatergic agents and dopamine receptors. Other clinically evaluated neuroprotective agents are, e.g., the monoamine oxidase B inhibitors selegiline and rasagiline, dopamine agonists, and the complex I mitochondrial fortifier coenzyme Q10;

antipsychotic medications: e.g., ziprasidone (Geodon™), risperidone (Risperdal™), and olanzapine (Zyprexa™);

agents used for multiple sclerosis such as beta-interferon (e.g., Avonex™, Betaseron™) Baclofen and Copaxone;

disease-modifying anti-rheumatic drugs (DMARDS) such as methotrexate, azathioptrine, leflunomide, pencillinamine, gold salts, mycophenolate mofetil, cyclophosphamide, CP-690,550; biological response modifiers (BRMs) such as Enbrel, Remicade, IL-1 antagonists; NSAIDS such as piroxicam, naproxen, indomethacin, ibuprofen and the like; COX-2 selective inhibitors such as Celebrex™; COX-1 inhibitors such as Feldene; immunosuppressives such as steroids, cyclosporine, Tacrolimus, rapamycin and the like;

PDE4 inhibitors such as theophylline, drotaverine hydrochloride, cilomilast, roflumilast, denbufylline, rolipram, tetomilast, enprofylline, arofylline, cipamfylline, tofimilast, filaminast, piclamilast, (R)-(+)-4-[2-(3-cyclopentyloxy-4-methoxyphenyl)-2-phenylethyl]pyridine, mesopram, N-(3,5-dichloro-4-pyridinyl)-2-[1-(4-fluorobenzyl)-5-hydroxy-1H-indol-3-yl]-2-oxoacetamide, CDC-801 (Celgene), CC-1088 (Celgene), Lirimilast, ONO-6126 (Ono), CC-10004 (Celgene) and MN-001 (Kyorin), ibudilast and pentoxifylline, for use in treating inflammation, lung disorders and as bronchodilators;

corticosteroids such as betamethasone, budesonide, cortisone, dexamethasone, hydrocortisone, methylprednisolone, prednisolone, prednisone and triamcinolone;

histamine H1 receptor antagonists such as bromopheniramine, chlorpheniramine, dexchlorpheniramine, triprolidine, clemastine, diphenhydramine, diphenylpyraline, tripelennamine, hydroxyzine, methdiazine, promethazine, trimeprazine, azatadine, cyproheptadine, antazoline, pheniramine pyrilamine, astemizole, terfenadine, loratadine, cetirizine, desloratadine, fexofenadine and levocetirizine;

histamine H2 receptor antagonists such as cimetidine, famotidine and ranitidine;

proton pump inhibitors such as omeprazole, pantoprazole and esomeprazole;

leukotriene antagonists and 5-lipoxygenase inhibitors such as zafirlukast, montelukast, pranlukast and zileuton;

nicotinic acetylcholine receptor agonists such as ABT-202, A-366833, ABT-594; BTG-102, A-85380, CGX1204;

P2X3 receptor antagonists such as A-317491, ISIS-13920, AZD-9056;

NGF agonists and antagonists such as RI-724, RI-1024, AMG-819, AMG-403, PPH 207;

NK1 and NK2 antagonists such as DA-5018, R-116301; CP-728663, ZD-2249;

NMDA antagonist such as NER-MD-11, CNS-5161, EAA-090, AZ-756, CNP-3381; potassium channel modulators such as CL-888, ICA-69673, retigabine;

GABA modulators such as lacosamide and propofol;

anti-cancer agents such as tyrosine kinase inhibitors imatinib (Gleevec/Glivec) and gefitinib (Iressa);

anti hyperlipidemia drugs such as statins, ezetimibe, niacin and bile acid sequestrants;

appetite suppressing agents: e.g., sibutramine, taranabant, rimobamant;

anti-diabetic medications such as insulin, tolbutamide (Orinase), acetohexamide (Dymelor), tolazamide (Tolinase), chlorpropamide (Diabinese), glipizide (Glucotrol), glyburide (Diabeta, Micronase, Glynase), glimepiride (Amaryl), gliclazide (Diamicron), repaglinide (Prandin), nateglinide (Starlix), pramlintide (Symlin) and exanatide (Byetla);

serotonergic and noradrenergic modulators such as SR-57746, paroxetine, duloxetine, clonidine, amitriptyline, citalopram, flibanserin; and

GI agents: e.g., laxatives (e.g., Lubiprostone (Amitiza), Fybogel®, Regulan®, Normacol® and the like), a gastrointestinal agent used for the treatment of idiopathic chronic constipation and constipation-predominant IBS, GI motility stimulants (e.g., domperidone, metoclopramide, mosapride, itopride), antispasmodic drugs (e.g., anticholinergics such as hyoscyamine or dicyclomine); anti-diarrheal medicines such loperamide (Imodium) and bismuth subsalicylate (as found in Pepto Bismol and Kaopectate), GCC (Guanylate Cyclase C) agonists (e.g., Linaclotide), 5HT4 agonists (e.g., Tegasarod), 5HT3 antagonists (e.g., Alosetron, Ramosetron, Ondansetron).

### Methods of Preparing the Compounds:

The compounds of Formula I may be prepared according to the schemes and examples depicted and described below. Unless otherwise specified, the starting materials and various intermediates may be obtained from commercial sources, prepared from commercially available compounds or prepared using well-known synthetic methods.

General Synthetic methods and General Synthetic Schemes

General synthetic procedures for the compounds of this invention are described below. The synthetic schemes are presented as examples and do not limit the scope of the invention in any way.

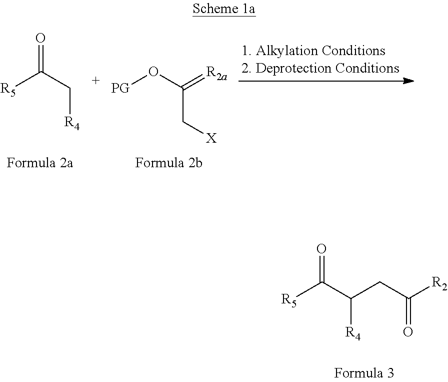

A compound of Formula 3 can be synthesized by the alkylation of a compound of Formula 2a by reaction with the appropriate protected (trapped) enolate compound of Formula 2b, wherein X is a leaving group and PG is an oxygen protecting group (Scheme 1a). Deprotection of the alcohol functionality (e.g., under acidic conditions), then releases the enolate, which tautomerizes in situ to the carbonyl compound of Formula 3.

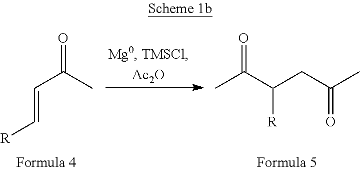

A compound of Formula 5 can be prepared from a compound of Formula 4 via the magnesium-mediated radical reduction of the α,β-unsaturation in the presence of trimethylsilyl chloride, followed by nucleophilic attack of the resulting β-carbanion onto an electrophile such as an acid chloride or anhydride. (Scheme 1b).

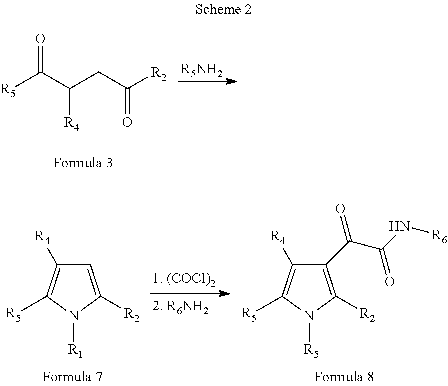

A compound of Formula 7 can be synthesized by the condensation reaction of a primary amine of general formula R⁶NH₂, and a 1,4-dicarbonyl moiety of Formula 3 (Scheme 2). A compound of Formula 8 can then be obtained from the intermediate of Formula 7 by electrophilic aromatic substitution at position three of the pyrrole ring using oxalyl chloride. This creates an intermediate α-keto acid chloride, which is subsequently aminated by a primary amine of general formula R₆NH₂ to produce a pyrrole of Formula 8.

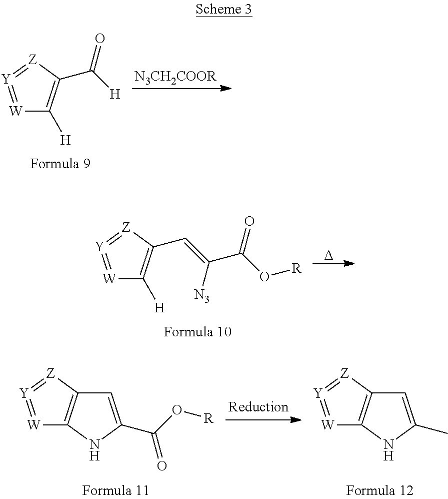

A compound of Formula 10 can be synthesized by the condensation of a compound of Formula 9 with an azidoacetate of general formula N₃CH₂C(O)OR in the presence of a strong alkaline base such as sodium methoxide (Scheme 3). In this scheme, W, Y and Z are independently selected from C, N, O or S. The alkylation of the aldehyde of formula 9 produces a β-hydroxy ester intermediate (not shown), which, upon subsequent in situ dehydration produces the vinyl azide of Formula 10. The resulting compound of formula 10 can be transformed to a [5,5]bicyclic product of Formula 11 via a Hemetsberger-Rees thermolysis reaction in toluene. Reduction of the ester functionality of a compound of Formula 11 to a methyl, using a reducing agent such as Lithium Aluminum Hydride, produces a compound of general Formula 12.

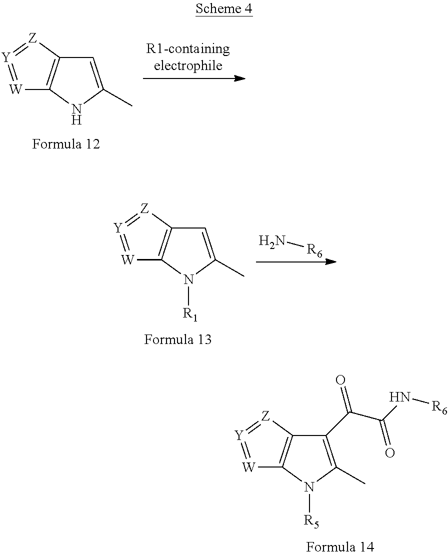

The pyrrole nitrogen of a compound of Formula 12 can be alkylated to form the corresponding compound of Formula 13 using an appropriate alkylating agent. As in Scheme 2, subsequent aromatic electrophilic substitution at position three of the pyrrole ring by oxalyl chloride creates an intermediate α-keto acid chloride, which can subsequently be aminated by a primary amine of the general formula R₆NH₂, to produce an α-keto amide compound of general Formula 14.

### General Analytical Techniques

LC/MS was run on a Waters Acquity system using a Polar C18 column, and 5 to 60% acetonitrile/water over 5 min. The ionization method for the MS was electrospray.

Automated column chromatography was run using an ISCO system. One of the Companion, Combiflash, or Combiflash Rf was used in each case.

Microwave reactions were run on a Personal Chemistry Optimizer, at 0-240° C., a power of 0-300 W and a pressure of 0-21 bar.

HPLC for purification was run on a Varian Prepstar instrument using the following conditions:

Solvent A: 0.1% Trifluoroacetic acid in water

Solvent B: 0.1% Trifluoroacetic acid in acetonitrile

| Time | % Solvent A | % Solvent B | Flow | Inject wait |
|---|---|---|---|---|
| 0:00 | 90 | 10 | 15 | x |
| 35:00 | 5 | 95 | 15 | |
| 43:00 | 5 | 95 | 15 | |
| 45:00 | 95 | 5 | 15 | |
| 50:00 | 98 | 2 | 0 | |

### EXAMPLES

All references provided in the Examples are herein incorporated by reference in their entirety. As used herein, all abbreviations, symbols and conventions are consistent with those used in the contemporary scientific literature. See, e.g., Janet S. Dodd, ed., The ACS Style Guide: A Manual for Authors and Editors, 2^{nd} Ed., Washington, D.C.: American Chemical Society, 1997, herein incorporated by reference in its entirety.

### Synthetic Procedures

### Example 1a

### Synthesis of 2-(2-oxopropyl)cyclooctanone (See Scheme 1a)

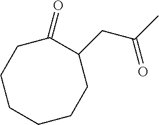

In a 50 mL round-bottomed flask equipped with a stir bar, NaH (484 mg, 12.1 mmol) was added to DMF (20 mL) under a nitrogen atmosphere. Cyclooctanone (1.39 g, 11 mmol) was added and the mixture was stirred at room temperature (RT) for 20 minutes. Then, 3-chloro-2-(methoxymethoxy)prop-1-ene (1.65 g, 12.1 mmol) was added, and the reaction mixture was heated at 120° C. for 2 hours, then allowed to cool to RT. Ice water (10 mL) was added, followed by EtOAc (50 mL). The layers were separated and the EtOAc layer was extracted with water (10 mL). The organic layers were combined, washed with brine, dried over Na₂SO₄, filtered and concentrated under reduced pressure. The crude mixture was dissolved in dioxane (5 mL), and H₂SO₄ (1%, 5 mL) was added. The reaction mixture was heated at 60° C. for 1 hour, followed by cooling to RT. Ice water (10 mL) was added, followed by EtOAc (50 mL). The layers were separated and the EtOAc layer was extracted with water (10 mL). The organic layers were combined, washed with brine, dried over Na₂SO₄, filtered and concentrated to afford 2-(2-oxopropyl)cyclooctanone which was used without further purification.

### Example 1b

### Synthesis of 3-phenylhexane-2,5-dione (See Scheme 1b)

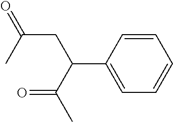

In a 250 mL round-bottomed flask equipped with a stir bar, (E)-4-phenylbut-3-en-2-one (1.4 g, 10 mmol) was dissolved in DMF (100 mL) under a nitrogen atmosphere. Acetic anhydride (2.8 mL, 30 mmol) and magnesium (0.72 g, 30 mmol) were added, followed by the dropwise addition of trimethylsilylchloride (3.78 ml, 30 mmol). The reaction was stirred overnight at RT, and ice water (10 mL) was then added, followed by EtOAc (50 mL). The layers were separated and the EtOAc layer was extracted with water (10 mL). The organic layers were combined, washed with brine, dried over Na₂SO₄, filtered and concentrated to afford 3-phenylhexane-2,5-dione as an oil. The product was used without further purification.

### Example 2

### Synthesis of 2-(1-(4-chlorobenzyl)-2-methyl-1,4,5,6,7,8-hexahydrocyclohepta[b]pyrrol-3-yl)-N-(3-chlorophenyl)-2-oxoacetamide (I-12) (See Scheme 2)

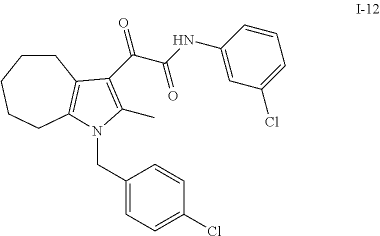 [SMILES: Cc1c(C(=O)C(=O)Nc2cccc(Cl)c2)c2c(n1Cc1ccc(Cl)cc1)CCCCC2]

In a 50 mL round-bottomed flask equipped with a stir bar, 4-chlorobenzylamine (1.3 mL, 11 mmol) and 2-(2-oxopropyl)cycloheptanone (1.84 g, 11 mmol) was stirred at ambient temperature. EtOAc (20 mL) was added and the organic layer was washed with water (3×50 mL). The organic layers were combined, washed with brine, dried over Na₂SO₄, filtered and concentrated. The crude mixture was subjected to flash chromatography on silica gel (0 to 50% EtOAc in hexanes) resulting in isolation of 1-(4-chlorobenzyl)-2-methyl-1,4,5,6,7,8-hexahydrocyclohepta[b]pyrrole. This was used directly in the next step without further purification.

In a 50 mL round-bottomed flask equipped with a stir bar was stirred 1-(4-chlorobenzyl)-2-methyl-1,4,5,6,7,8-hexahydrocyclohepta[b]pyrrole (800 mg, 2.9 mmol) in DCM (10 mL) at −60° C. under a nitrogen atmosphere. Oxalyl chloride (1.46 mL of a 2M DCM solution, 2.93 mmol) was added, the mixture warmed to RT and stirred at this temperature for 15 minutes then concentrated under vacuum. The resulting crude acid chloride (530 mg, 1.46 mmol) was placed under high vacuum for 2 hours, and was then dissolved in DCM (5 mL), and cooled to −10° C. under a nitrogen atmosphere.

Triethylamine (0.49 mL, 3.5 mmol) and 3-chloroaniline (259 mg, 2.04 mmol) were added to the above solution and the reaction mixture was stirred at RT for 3 hours. Water (20 mL) was added and the DCM layer was separated. Water (15 mL) and DCM (10 mL) were added and the water layer was extracted with more DCM (10 mL). The organic layers were combined, washed with brine, dried over Na₂SO₄, filtered and concentrated. The crude mixture was subjected to flash chromatography on silica gel (0 to 50% EtOAc in hexanes) resulting in isolation of Compound I-12 as a yellow solid. ¹H NMR (CDCl₃/400 MHz) δ 8.80 (s, 1H), 7.80 (t, 1H), 7.46 (d, 1H), 7.28 (m, 3H), 7.12 (d, 1H), 6.87 (d, 2H), 4.90 (s, 2H), 2.68-2.65 (m, 2H), 2.50-2.45 (m, 2H), 2.35 (s, 3H), 1.80-1.50 (m, 6H); MS m/z: 454 (M+1).

### Example 3

### Synthesis of 5-methyl-6H-thieno[2,3-b]pyrrole (See Scheme 3)

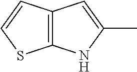

In a 3 L round-bottomed flask equipped with a stir bar, methanol (1.49 L), thiophene-3-carboxaldehyde (50 g, 446 mmol) and methyl 2-azidoacetate (205 g, 1783 mmol) were combined and the resulting mixture cooled to −25° C. Sodium methoxide (385 mL, 1783 mmol, 25% w/w methanol) was added dropwise at this temperature over 3.5 hours. The reaction mixture was allowed to warm to RT over the course of 2 h, then it was poured over ammonium chloride (95 g) in ice water. This mixture was stored in the freezer overnight. Methanol was removed in vacuo, water (500 mL) was added, followed by EtOAc (500 mL). The layers were separated and the EtOAc layer was further extracted with water (10 mL). The organic layers were combined, washed with brine, dried over Na₂SO₄, filtered through a pad of celite and concentrated. The product (Z)-methyl 2-azido-3(thiophen-3-yl)acrylate was used in the next step without further purification.

In a 2 L round-bottomed flask equipped with a stir bar, (Z)-methyl-2-azido-3-(thiophen-3-yl)acrylate (36.3 g, 173 mmol) in toluene (450 mL) was heated at reflux for 75 minutes. The mixture was concentrated, and a 4:1 hexanes/DCM mixture was added until a product had precipitated from the solution. The solid was collected, dissolved in minimal DCM and flushed through a pad of silica gel to yield methyl 6H-thieno[2,3-b]pyrrole-5-carboxylate as a tan solid. This was used in the next step without further purification.

In a 200 mL round-bottomed flask equipped with a stir bar, 6H-thieno[2,3-b]pyrrole-5-carboxylate (20.2 g, 111 mmol) was dissolved in THF (50 mL) and cooled to 0° C. Lithium aluminum hydride (1.0 N in THF, 330 mL, 330 mmol) was added slowly (caution, gas evolution can be intense). The resulting mixture was heated at reflux for 22 hours, then was cooled to 0° C. and quenched by slow addition of water (12 mL), 4N NaOH (12 mL), and water (36 mL). This mixture was stirred for 30 minutes, followed by addition of MgSO₄ (4 g) and stirring for an additional 30 minutes. The mixture was filtered, concentrated and the resulting crude product was dissolved in EtOAc, dried over MgSO₄, filtered and concentrated. The product was azeotroped with toluene (3×50 mL) resulting in isolation of 5-methyl-6H-thieno[2,3-b]pyrrole as a light brown solid.

### Example 4

### Synthesis of 2-(6-(4-chlorobenzyl)-5-methyl-6H-thieno[2,3-b]pyrrol-4-yl)-N-(3-chlorophenyl)-2-oxoacetamide (I-08) (See Scheme 4)

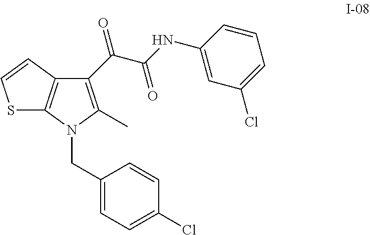 [SMILES: Cc1c(C(=O)C(=O)Nc2cccc(Cl)c2)c2ccsc2n1Cc1ccc(Cl)cc1]

In a 50 mL round-bottomed flask equipped with a stir bar was stirred DMF (10 mL) and NaH (80 mg, 2.0 mmol, 60%) under a nitrogen atmosphere. Then, 5-methyl-6H-thieno[2,3-b]pyrrole (274 mg, 2.0 mmol) was added and the mixture stirred for 15 minutes at 0° C. Next, 4-chlorobenzylbromide (493 mg, 2.4 mmol) was added, then the reaction stirred for 2 hours at 0° C., followed by stirring at RT overnight. Ice water (10 mL) was added, followed by EtOAc (20 mL). The layers were separated and the EtOAc layer was extracted with water (10 mL). The organic layers were combined, washed with brine, dried over Na₂SO₄, filtered and concentrated. The crude mixture was subjected to flash chromatography on silica gel (0 to 50% EtOAc in hexanes) resulting in isolation of 6-benzyl-5-methyl-6H-thieno[2,3-b]pyrrole as a yellow solid. This intermediate was used directly in the next step without further purification.

In a 50 mL round-bottomed flask equipped with a stir bar was stirred 6-benzyl-5-methyl-6H-thieno[2,3-b]pyrrole (522 mg, 1.0 mmol) and DCM (10 mL) at −60° C., under a nitrogen atmosphere. Oxalyl chloride (1 mL of a 2M DCM solution, 2 mmol) was added and the solution was stirred at RT for 15 minutes, then it was concentrated under vacuum. The resulting crude acid chloride (243 mg, 1 mmol) was placed under high vacuum for 2 hours and was then dissolved in DCM (5 mL) and cooled to −10° C. under a nitrogen atmosphere.

Triethylamine (0.335 mL, 2.4 mmol) and 3-chloroaniline (127 mg, 1 mmol) were added to the above solution and the reaction mixture was stirred at RT for 3 hours. Water (20 mL) was added and the DCM layer was separated. Water (15 mL) and DCM (10 mL) were added again and the water layer was extracted with DCM (10 mL) once more. The organic layers were combined, washed with brine, dried over Na₂SO₄, filtered and concentrated. The crude mixture was subjected to flash chromatography over silica gel (0 to 50% EtOAc in hexanes) resulting in isolation of Compound I-08 (100 mg, 23%) as a yellow solid. ¹H NMR (CDCl₃/400 MHz) δ 9.15 (s, 1H), 7.90 (t, 1H), 7.60 (d, 1H), 7.53 (d, 1H), 7.30 (m, 3H), 7.16 (d, 1H), 7.08 (d, 2H), 6.94 (d, 2H), 5.20 (s, 2H), 2.74 (s, 3H); MS m/z: 443 (M+1).

The following compounds were prepared according to Schemes 1A or 1B and Scheme 2:

### 2-(1-(4-chlorobenzyl)-2-methyl-4,5,6,7,8,9-hexahydro-1H-cycloocta[b]pyrrol-3-yl)-N-(3-chlorophenyl)-2-oxoacetamide (I-10)

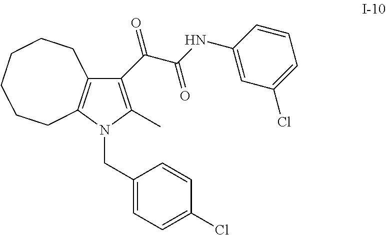 [SMILES: Cc1c(C(=O)C(=O)Nc2cccc(Cl)c2)c2c(n1Cc1ccc(Cl)cc1)CCCCCC2]

I-10 was prepared according to Schemes 1a and 2. ¹H NMR (CDCl₃/400 MHz) δ 8.81 (s, 1H), 7.82 (d, 1H), 7.50 (d, 1H), 7.30 (d, 2H), 7.14 (d, 1H), 6.88 (d, 2H), 5.01 (s, 2H), 2.78 (t, 2H), 2.57-2.54 (m, 2H), 2.36 (s, 3H), 1.68-1.60 (m, 2H), 1.42-1.34 (m, 6H); MS m/z: 468 (M+1).

### 2-(1-(4-chlorobenzyl)-2-methyl-4,5,6,7,8,9-hexahydro-1H-cycloocta[b]pyrrol-3-yl)-N-(2-methoxypyridin-4-yl)-2-oxoacetamide (I-11)

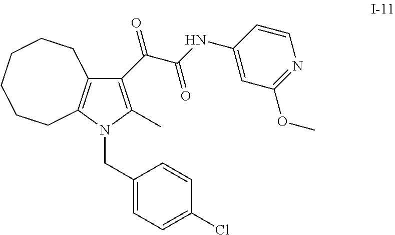 [SMILES: COc1cc(NC(=O)C(=O)c2c3c(n(Cc4ccc(Cl)cc4)c2C)CCCCCC3)ccn1]

I-11 was prepared according to Schemes 1a and 2. ¹H NMR (CDCl₃/400 MHz) δ 8.82 (s, 1H), 8.10 (d, 1H), 7.29 (d, 2H), 7.18 (d, 1H), 7.10 (d, 2H), 6.85 (d, 2H), 5.02 (s, 2H), 3.84 (s, 3H), 2.78-2.70 (m, 2H), 2.59-2.51 (m, 2H), 2.34 (s, 3H), 1.68-1.60 (m, 2H), 1.40-1.30 (m, 6H); MS m/z: 466 (M+1).

### 2-(1-(4-chlorobenzyl)-2-methyl-1,4,5,6,7,8-hexahydrocyclohepta[b]pyrrol-3-yl)-N-(2-methoxypyridin-4-yl)-2-oxoacetamide (I-13)

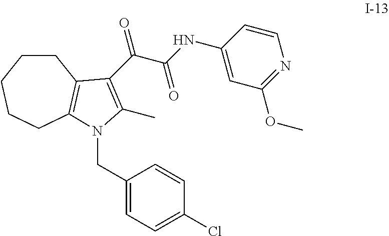 [SMILES: COc1cc(NC(=O)C(=O)c2c3c(n(Cc4ccc(Cl)cc4)c2C)CCCCC3)ccn1]

I-13 was prepared according to Schemes 1a and 2. ¹H NMR (CDCl₃/400 MHz) δ 8.94 (s, 1H), 8.03 (d, 1H), 7.28 (d, 2H), 7.19 (d, 1H), 7.15 (d, 2H), 6.84 (d, 2H), 5.0 (s, 2H), 3.92 (s, 3H), 2.70-2.65 (m, 2H), 2.50-2.45 (m, 2H), 2.30 (s, 3H), 1.78-1.70 (m, 2H), 1.68-1.62 (m, 2H), 1.60-1.55 (m, 2H); MS m/z: 451 (M+1).

### 2-(1-(4-chlorobenzyl)-2,5,5-trimethyl-4,5,6,7-tetrahydro-1H-indol-3-yl)-N-(3-chlorophenyl)-2-oxoacetamide (I-14)

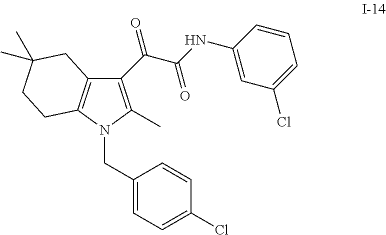 [SMILES: Cc1c(C(=O)C(=O)Nc2cccc(Cl)c2)c2c(n1Cc1ccc(Cl)cc1)CCC(C)(C)C2]

I-14 was prepared according to Schemes 1a and 2. ¹H NMR (CDCl₃/400 MHz) δ 8.76 (s, 1H), 7.80 (t, 1H), 7.48 (d, 1H), 7.28 (m, 3H), 7.12 (d, 1H), 6.85 (d, 2H), 4.90 (s, 2H), 2.47 (s, 2H), 2.38 (s, 3H), 2.32 (t, 2H), 1.52 (t, 2H), 0.92 (s, 6H); MS m/z: 468 (M+1).

### 2-(1-(4-chlorobenzyl)-2,5,5-trimethyl-4,5,6,7-tetrahydro-1H-indol-3-yl)-N-(2-methoxypyridin-4-yl)-2-oxoacetamide (I-15)

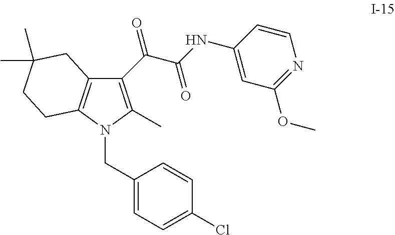

I-15 was prepared according to Schemes 1a and 2. ¹H NMR (CDCl₃/400 MHz) δ 8.76 (s, 1H), 8.04 (d, 1H), 7.23 (d, 2H), 7.13 (d, 1H), 7.04 (d, 2H), 6.81 (d, 2H), 4.93 (s, 2H), 3.88 (s, 2H), 2.40 (s, 3H), 2.28 (t, 2H), 1.49 (t, 2H), 0.86 (s, 6H); MS m/z: 465 (M+1).

### 2-(1-(4-chlorobenzyl)-2-methyl-4,5,6,7-tetrahydro-1H-indol-3-yl)-N-(3-chlorophenyl)-2-oxoacetamide (I-16)

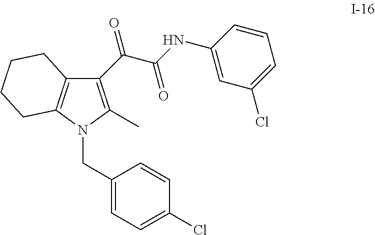 [SMILES: Cc1c(C(=O)C(=O)Nc2cccc(Cl)c2)c2c(n1Cc1ccc(Cl)cc1)CCCC2]

I-16 was prepared according to Schemes 1a and 2. ¹H NMR (CDCl₃/400 MHz) δ 8.79 (s, 1H), 7.80 (t, 1H), 7.48 (d, 1H), 7.28 (m, 3H), 7.12 (d, 1H), 6.87 (d, 2H), 4.90 (s, 2H), 2.66 (t, 2H), 2.39 (s, 3H), 2.35 (t, 2H), 1.80-1.74 (m, 2H), 1.70-1.64 (m, 2H); MS m/z: 440 (M+1).

### 2-(1-(4-chlorobenzyl)-2-methyl-4,5,6,7-tetrahydro-1H-indol-3-yl)-N-(2-methoxypyridin-4-yl)-2-oxoacetamide (I-18)

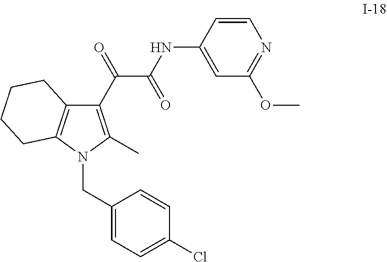 [SMILES: COc1cc(NC(=O)C(=O)c2c3c(n(Cc4ccc(Cl)cc4)c2C)CCCC3)ccn1]

I-18 was prepared according to Schemes 1a and 2. ¹H NMR (CDCl₃/400 MHz) δ 8.94 (s, 1H), 8.03 (d, 1H), 7.28 (d, 2H), 7.19 (d, 1H), 7.15 (d, 2H), 6.86 (d, 2H), 4.90 (s, 2H), 3.90 (s, 3H), 2.62 (t, 2H), 2.38 (s, 3H), 2.32 (t, 2H), 1.78-1.72 (m, 2H), 1.68-1.62 (m, 2H); MS m/z: 438 (M+1).

### 2-(1-(4-fluorobenzyl)-2,5-dimethyl-1H-pyrrol-3-yl)-N-(2-methoxypyridin-4-yl)-2-oxoacetamide (I-17)

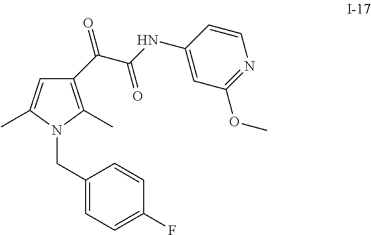

I-17 was prepared according to Scheme 2. ¹H NMR (CD₃OD/400 MHz) δ 8.02 (dd, 1H), 7.31 (d, 1H), 7.24 (dd, 1H), 7.08-7.04 (m, 2H), 6.98-6.94 (m, 2H), 6.74 (d, 1H), 5.17 (s, 2H), 3.89 (s, 3H), 2.51 (s, 3H), 2.14 (s, 3H).

### 2-(1-(4-chlorobenzyl)-2,5-dimethyl-4-phenyl-1H-pyrrol-3-yl)-N-(2-methoxypyridin-4-yl)-2-oxoacetamide (I-19)

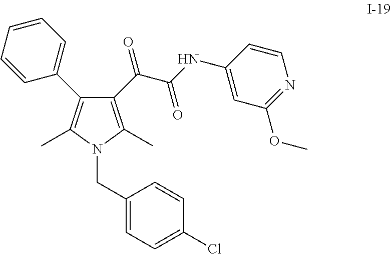 [SMILES: COc1cc(NC(=O)C(=O)c2c(-c3ccccc3)c(C)n(Cc3ccc(Cl)cc3)c2C)ccn1]

I-19 was prepared according to Schemes 1b and 2. ¹H NMR (CDCl₃/400 MHz) δ 7.82 (d, 1H), 7.43 (m, 3H), 7.35 (d, 2H), 7.29 (d, 2H), 7.09 (s, 1H), 6.88 (d, 2H), 6.57 (d, 1H), 6.42 (d, 1H), 5.14 (s, 2H), 3.81 (s, 3H), 2.57 (s, 3H), 2.0 (s, 3H); MS m/z: 474 (M+1).

### 2-(1-(4-chlorobenzyl)-2,5-dimethyl-4-phenyl-1H-pyrrol-3-yl)-N-(3-chlorophenyl)-2-oxoacetamide (I-20)

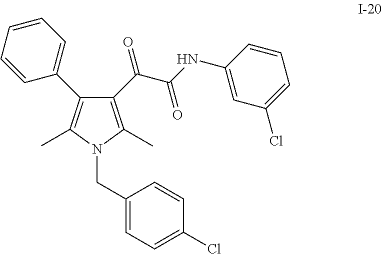 [SMILES: Cc1c(C(=O)C(=O)Nc2cccc(Cl)c2)c(-c2ccccc2)c(C)n1Cc1ccc(Cl)cc1]

I-20 was prepared according to Schemes 1b and 2. ¹H NMR (CDCl₃/400 MHz) δ 7.35-7.25 (m, 9H), 7.40 (d, 2H), 6.88 (d, 2H), 5.08 (s, 2H), 4.42 (s, 1H), 2.49 (s, 3H), 2.0 (s, 3H); MS m/z: 476 (M+1).

### 2-(1-(2,4-dichlorobenzyl)-2,5-dimethyl-1H-pyrrol-3-yl)-N-(2-methoxypyridin-4-yl)-2-oxoacetamide (I-21)

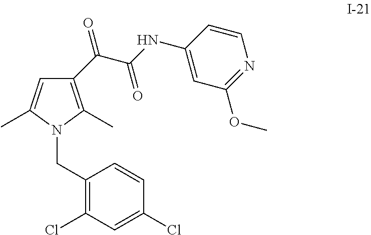

I-21 was prepared according to Scheme 2.

### 2-(1-(4-methoxybenzyl)-2,5-dimethyl-1H-pyrrol-3-yl)-N-(2-methoxypyridin-4-yl)-2-oxoacetamide (I-22)

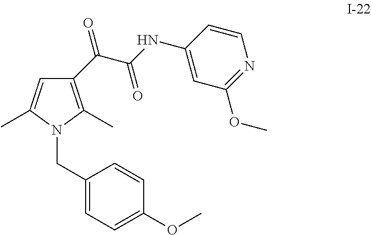

I-22 was prepared according to Scheme 2. ¹H NMR (CDCl₃/400 MHz) δ 9.23 (s, 1H), 8.10 (d, 1H), 7.17-7.16 (m, 1H), 7.14-7.11 (m, 2H), 6.824-6.79 (m, 5H), 4.99 (s, 2H), 3.90 (s, 3H), 3.74 (d, 3H), 2.51 (s, 3H), 2.13 (s, 3H).

### 2-(1-(4-chlorobenzyl)-5-methyl-2-phenyl-1H-pyrrol-3-yl)-N-(2-methoxypyridin-4-yl)-2-oxoacetamide (I-23)

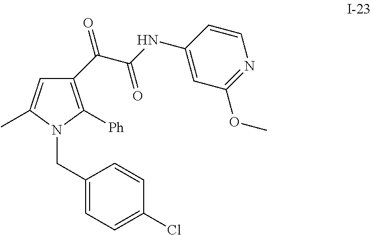

I-23 was prepared according to Schemes 1a and 2. ¹H NMR (CDCl₃/400 MHz) δ 9.13 (s, 1H), 8.08 (d, 1H), 7.40-7.03 (m, 10H), 6.79 (d, 2H), 4.92 (s, 2H), 3.93 (s, 3H), 2.16 (s, 3H).

### 2-(1-(4-chlorobenzyl)-5-methyl-2-phenyl-1H-pyrrol-3-yl)-N-(3-chlorophenyl)-2-oxoacetamide (I-24)

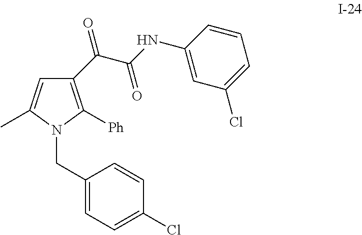 [SMILES: Cc1cc(C(=O)C(=O)Nc2cccc(Cl)c2)c(-c2ccccc2)n1Cc1ccc(Cl)cc1]

I-24 was prepared according to Schemes 1a and 2. ¹H NMR (CDCl₃/400 MHz) δ 9.08 (s, 1H), 7.80 (m, 1H), 7.40-7.10 (m, 11H), 6.79 (d, 2H), 4.92 (s, 2H), 3.93 (s, 3H), 2.16 (s, 3H).

### 2-(1-(4-chlorobenzyl)-2-methyl-5-phenyl-1H-pyrrol-3-yl)-N-(3-chlorophenyl)-2-oxoacetamide (I-25)

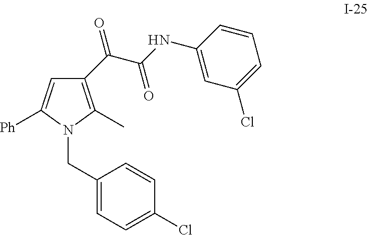 [SMILES: Cc1c(C(=O)C(=O)Nc2cccc(Cl)c2)cc(-c2ccccc2)n1Cc1ccc(Cl)cc1]

I-25 was prepared according to Scheme 2. ¹H NMR (CDCl₃/400 MHz) δ 9.24 (s, 1H), 7.87 (s, 1H), 7.55 (s, 1H), 7.49 (d, 1H), 7.33-7.25 (m, 8H), 7.12 (d, 1H), 6.86 (d, 2H), 5.14 (s, 2H), 2.54 (s, 3H).

### 2-(1-(4-chlorobenzyl)-2-methyl-5-phenyl-1H-pyrrol-3-yl)-2-oxo-N-(pyridin-4-yl)acetamide (I-26)

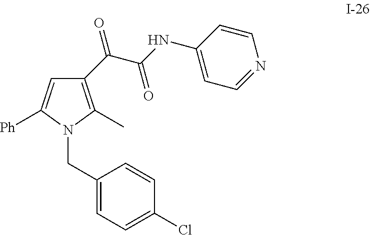 [SMILES: Cc1c(C(=O)C(=O)Nc2ccncc2)cc(-c2ccccc2)n1Cc1ccc(Cl)cc1]

I-26 was prepared according to Scheme 2. ¹H NMR (CDCl₃/400 MHz) δ 9.41 (s, 1H), 8.55 (d, 2H), 7.64-7.62 (m, 2H), 7.51 (s, 1H), 7.34-7.25, (m, 7H), 6.84 (d, 2H), 5.13 (s, 2H), 2.54 (3H).

### 2-(1-(4-chlorobenzyl)-2-methyl-5-phenyl-1H-pyrrol-3-yl)-N-(2-methoxypyridin-4-yl)-2-oxoacetamide (I-27)

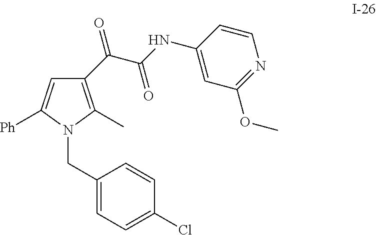 [SMILES: COc1cc(NC(=O)C(=O)c2cc(-c3ccccc3)n(Cc3ccc(Cl)cc3)c2C)ccn1]

I-27 was prepared according to Scheme 2. ¹H NMR (CDCl₃/400 MHz) δ 9.40 (bs, 1H), 8.13 (d, 1H), 7.50 (s, 1H), 7.34-7.25 (m, 8H), 7.18 (d, 1H), 6.85 (d, 2H), 5.14 (s, 2H), 3.99 (s, 3H), 2.54 (s, 3H).

### 2-(1-(4-chlorobenzyl)-2,5-dimethyl-1H-pyrrol-3-yl)-N-(2-chloropyridin-4-yl)-2-oxoacetamide (I-28)

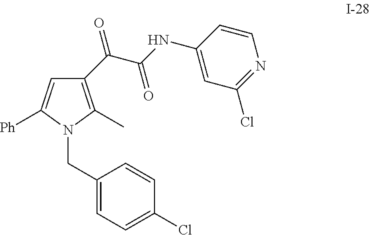 [SMILES: Cc1c(C(=O)C(=O)Nc2ccnc(Cl)c2)cc(-c2ccccc2)n1Cc1ccc(Cl)cc1]

I-28 was prepared according to scheme 2. ¹H NMR (CDCl₃/400 MHz) δ 9.45 (bs, 1H), 8.31 (d, 1H), 7.81 (s, H), 7.46 (d, 1H), 7.29 (d, 2H), 7.17 (s, 1H), 6.84 (d, 2H), 5.06 (s, 2H), 2.52 (s, 3H), 2.15 (s, 3H).
2-(1-(4-chlorobenzyl)-2,5-dimethyl-1H-pyrrol-3-yl)-N-(3-chlorophenyl)-2-oxoacetamide (I-29).

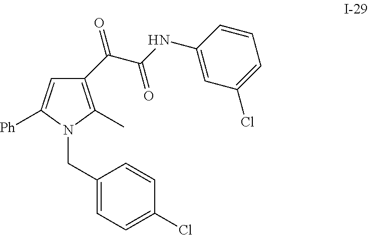 [SMILES: Cc1c(C(=O)C(=O)Nc2cccc(Cl)c2)cc(-c2ccccc2)n1Cc1ccc(Cl)cc1]

I-29 was prepared according to Scheme 2. ¹H NMR (CDCl₃/400 MHz) δ 9.24 (bs, 1H), 7.87 (s, 1H), 7.48 (d, 1H), 7.31-7.22 (m, 4H), 7.12 (d, 1H), 6.84 (d, 2H), 5.06 (s, 2H), 2.53 (s, 3H), 2.15 (s, 3H).

### 2-(1-(4-chlorobenzyl)-2,5-dimethyl-1H-pyrrol-3-yl)-2-oxo-N-(pyridin-4-yl)acetamide (I-30)

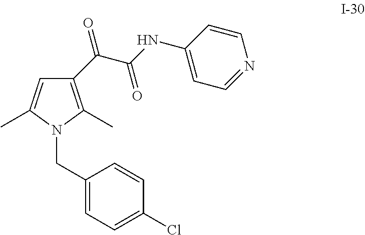 [SMILES: Cc1cc(C(=O)C(=O)Nc2ccncc2)c(C)n1Cc1ccc(Cl)cc1]

I-30 was prepared according to Scheme 2. ¹H NMR (CDCl₃/400 MHz) δ 9.38 (bs, 1H), 8.56 (m, 2H), 7.61 (d, 2H), 7.29 (d, 2H), 6.84 (d, 2H), 5.06 (s, 2H), 2.53 (s, 3H), 2.15 (s, 3H).

### 2-(1-(4-chlorobenzyl)-2,5-dimethyl-1H-pyrrol-3-yl)-2-oxo-N-(pyridin-3-yl)acetamide (I-31)

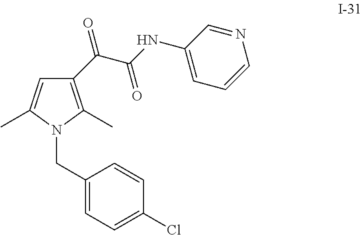 [SMILES: Cc1cc(C(=O)C(=O)Nc2cccnc2)c(C)n1Cc1ccc(Cl)cc1]

I-31 was prepared according to Scheme 2. ¹H NMR (CDCl₃/400 MHz) δ 9.32 (bs, 1H), 8.78 (bs, 1H), 8.40 (d, 1H), 8.29 (d, 1H), 7.35-7.21 (m, 4H), 6.84 (d, 2H), 5.06 (s, 2H), 2.54 (s, 3H), 2.15 (s, 3H);

### 2-(1-(4-chlorobenzyl)-2,5-dimethyl-1H-pyrrol-3-yl)-2-oxo-N-phenylacetamide (I-32)

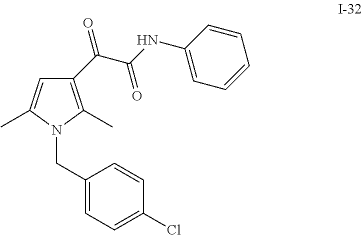 [SMILES: Cc1cc(C(=O)C(=O)Nc2ccccc2)c(C)n1Cc1ccc(Cl)cc1]

I-32 was prepared according to Scheme 2. ¹H NMR (CDCl₃/400 MHz) δ 9.20 (bs, 1H), 7.69 (dd, 2H), 739-7.35 (m, 2H), 7.30-7.23 (m, 3H), 7.20-7.15 (m, 1H), 6.85 (d, 2H), 5.05 (s, 2H), 2.54 (s, 3H), 2.14 (d, 3H)

### 2-(1-(4-chlorobenzyl)-2,5-dimethyl-1H-pyrrol-3-yl)-N-(2-methoxypyridin-4-yl)-2-oxoacetamide (I-01)

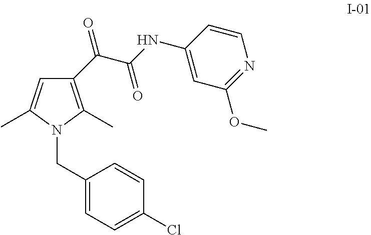

I-01 was prepared according to Scheme 2. ¹H NMR (CDCl₃/400 MHz) δ 9.27 (bs, 1H), 8.10 (d, 1H), 7.29-7.27 (m, 2H), 7.18 (bs, 2H), 7.13 (dd, 1H), 6.83 (d, 2H), 5.05 (s, 2H), 3.93 (s, 3H), 2.52 (s, 3H), 2.14 (d, 3H).

The Following Compounds were Prepared According to Scheme 3 and Scheme 4:

### 2-(4-(4-chlorobenzyl)-2,5-dimethyl-4H-thieno[3,2-b]pyrrol-6-yl)-2-oxo-N-(pyridin-4-yl)acetamide (I-04)

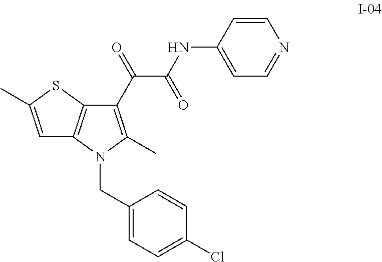 [SMILES: Cc1cc2c(s1)c(C(=O)C(=O)Nc1ccncc1)c(C)n2Cc1ccc(Cl)cc1]

¹H NMR (CDCl₃/400 MHz) δ 9.50 (s, 1H), 8.58 (d, 2H), 7.68 (d, 2H), 7.30 (d, 2H), 7.00 (d, 2H), 6.55 (s, 1H), 5.22 (s, 2H), 2.72 (s, 3H), 2.50 (s, 3H); MS m/z: 424 (M+1).

### 2-(4-(4-chlorobenzyl)-2,5-dimethyl-4H-thieno[3,2-b]pyrrol-6-yl)-N-(2-methoxypyridin-4-yl)-2-oxoacetamide (I-05)

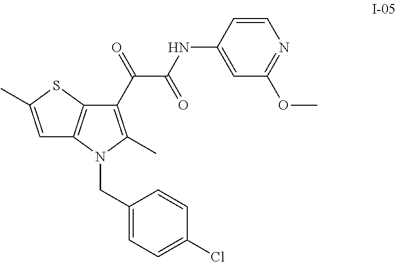

¹H NMR (CDCl₃/400 MHz) δ 9.40 (s, 1H), 8.12 (d, 1H), 7.30 (d, 2H), 7.22 (m, 2H), 6.98 (d, 2H), 6.54 (s, 1H), 5.20 (s, 2H), 3.90 (s, 3H), 2.70 (s, 3H), 2.50 (s, 3H); MS m/z: 454 (M+1).

### 2-(4-(4-chlorobenzyl)-5-methyl-4H-thieno[3,2-b]pyrrol-6-yl)-N-(3-chlorophenyl)-2-oxoacetamide (I-06)

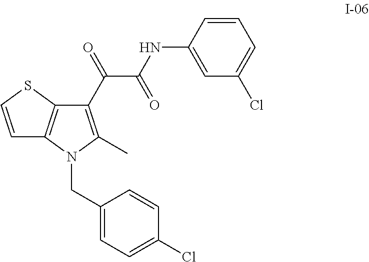 [SMILES: Cc1c(C(=O)C(=O)Nc2cccc(Cl)c2)c2sccc2n1Cc1ccc(Cl)cc1]

¹H NMR (CDCl₃/400 MHz) δ 9.40 (s, 1H), 7.58 (d, 1H), 7.34-7.30 (m, 3H), 7.22 (m, 2H), 7.22-7.15 (m, 3H), 7.07 (t, 1H), 7.01 (d, 2H), 6.86 (d, 1H), 5.31 (s, 2H), 2.76 (s, 3H); MS m/z: 443 (M+1).

### 2-(4-(4-chlorobenzyl)-5-methyl-4H-thieno[3,2-b]pyrrol-6-yl)-N-(2-methoxypyridin-4-yl)-2-oxoacetamide (I-07)

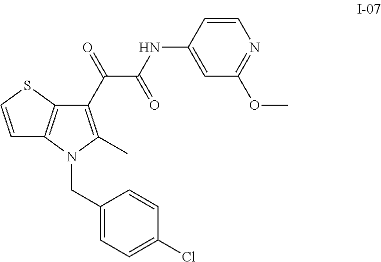 [SMILES: COc1cc(NC(=O)C(=O)c2c(C)n(Cc3ccc(Cl)cc3)c3ccsc23)ccn1]

¹H NMR (CDCl₃/400 MHz) δ 9.42 (s, 1H), 8.20 (d, 1H), 7.39 (d, 3H), 7.30-7.20 (m, 3H), 7.09 (d, 2H), 6.88 (d, 1H), 5.38 (s, 2H), 4.00 (s, 3H), 3.80 (s, 3H); MS m/z: 440 (M+1).

### 2-(6-(4-chlorobenzyl)-5-methyl-6H-thieno[2,3-b]pyrrol-4-yl)-N-(2-methoxypyridin-4-yl)-2-oxoacetamide (I-09)

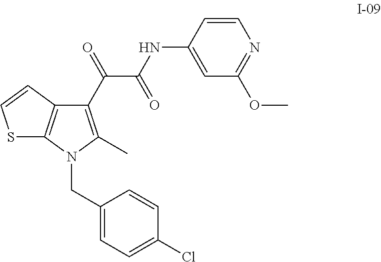 [SMILES: COc1cc(NC(=O)C(=O)c2c(C)n(Cc3ccc(Cl)cc3)c3sccc23)ccn1]

¹H NMR (CDCl₃/400 MHz) δ 9.20 (s, 1H), 8.12 (d, 1H), 7.59 (d, 1H), 7.32 (d, 2H), 7.26 (d, 2H), 7.18 (d, 1H), 7.09 (d, 2H), 6.95 (d, 2H), 5.20 (s, 2H), 3.92 (s, 3H), 2.72 (s, 3H); MS m/z: 440 (M+1).

The Following Compounds were Prepared According to Scheme 4:

### 2-(2-chloro-6-(4-chlorobenzyl)-5-methyl-6H-thieno[2,3-b]pyrrol-4-yl)-2-oxo-N-(pyridin-4-yl)acetamide (I-02)

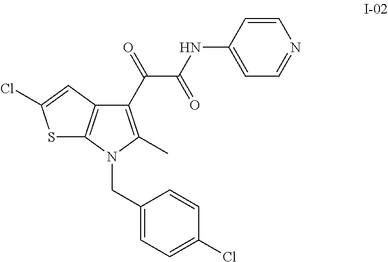 [SMILES: Cc1c(C(=O)C(=O)Nc2ccncc2)c2cc(Cl)sc2n1Cc1ccc(Cl)cc1]

I-02 was prepared according to Scheme 4 with the exception that methyl 6H-thieno[2,3-b]pyrrole-5-carboxylate was chlorinated under the following general conditions:

Methyl 6H-thieno[2,3-b]pyrrole-5-carboxylate (1.0 equiv) and N-chlorosuccinimide (1.05 equiv.) were dissolved in CHCl₃ and stirred overnight. The mixture was neutralized to pH 7 with 4N NaOH (aq.), followed by extraction with EtOAc. The organic layer was washed with brine, dried over Na₂SO₄, filtered and concentrated to give methyl 2-chloro-6H-thieno[2,3-b]pyrrole-5-carboxylate. ¹H NMR (CDCl₃/400 MHz) δ 9.25 (s, 1H), 8.60 (d, 2H), 7.64 (d, 2H), 7.55 (s, 1H), 7.38 (d, 2H), 7.10 (d, 2H), 5.18 (s, 2H), 2.75 (s, 3H); MS m/z: 444 (M+1).

### 2-(2-chloro-6-(4-chlorobenzyl)-5-methyl-6H-thieno[2,3-b]pyrrol-4-yl)-N-(2-methoxypyridin-4-yl)-2-oxoacetamide (I-03)

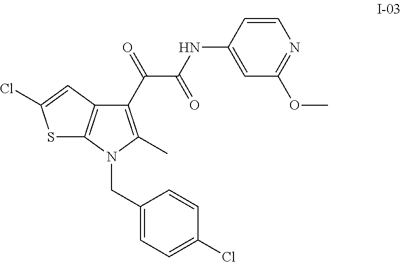 [SMILES: COc1cc(NC(=O)C(=O)c2c(C)n(Cc3ccc(Cl)cc3)c3sc(Cl)cc23)ccn1]

I-03 was prepared according to Scheme 4 with the exception that methyl 6H-thieno[2,3-b]pyrrole-5-carboxylate was chlorinated under the following gerenal conditions:

Methyl 6H-thieno[2,3-b]pyrrole-5-carboxylate (1.0 equiv) and N-chlorosuccinimide (1.05 equiv.) were dissolved in CHCl₃ and stirred overnight. The mixture was neutralized to pH 7 with 4N NaOH (aq.), followed by extraction with EtOAc. The organic layer was washed with brine, dried over Na₂SO₄, filtered and concentrated to give methyl 2-chloro-6H-thieno[2,3-b]pyrrole-5-carboxylate. ¹H NMR (CDCl₃/400 MHz) δ 9.20 (s, 1H), 8.18 (d, 1H), 7.52 (s, 1H), 7.34 (d, 2H), 7.24 (d, 1H), 7.14 (m, 1H), 7.08 (d, 2H), 5.18 (s, 2H), 3.90 (s, 3H), 2.70 (s, 3H); MS m/z: 474 (M+1).

### Biological Assays:

### Example 5

### FAAH Inhibition Using Rat, Mouse and Human Brain Homogenate Assays

The ability of compounds to inhibit FAAH was measured in human whole cell and human and rodent brain homogenates as described herein.

A. FAAH Rat Brain Membrane (RBM) Homogenate Preparation

Adult rats (Charles River CD strain, female, 200 g) were anaesthetized with isofluorane and rapidly decapitated. Each brain was quickly removed and chilled in tubes (3 brains per tube) on ice. About 25 mL of “homogenization buffer” (20 mM HEPES buffer, pH 7.0, with 1 mM MgCl₂) was added to 15 to 20 g of brain. The brains were homogenized on ice for 1 minute using an Omni GLH homogenizer (Omni International, Marietta, Ga.). The homogenates were then transferred to three centrifuge tubes and centrifuged at 36,500 g for 20 minutes at 4° C. The supernatant was discarded and each pellet was re-suspended in 25 mL homogenization buffer. The re-suspended material was again centrifuged (36,500 g, 20 minutes at 4° C.). Pellets were combined by resuspension in 10 mL of homogenization buffer and incubated in a 37° C. water bath for 15 minutes. The tubes were then placed on ice for 5 minutes followed by centrifugation at 36,500 g for 20 minutes at 4° C. The supernatant was discarded and the membrane pellets were then re-suspended in 40 mL of “resuspension buffer” (50 mM Tris-HCl buffer, pH 7.4, containing 1 mM EDTA and 3 mM MgCl₂). A Bradford Protein assay was performed to determine protein concentration. The protein was aliquotted into screw cap Cryo tubes each containing ˜400 μL, flash frozen in liquid nitrogen and stored at −80° C. until used for the assay. A similar protocol was used to obtain brain membrane homogenates from mice.

B. FAAH Human Brain Membrane (HBM) Homogenate Preparation

About 10 g of frozen normal human brain cortex tissue was obtained (e.g., from Analytical Biological Services (ABS), Inc. (Wilmington, Del.)). The brain tissue was thawed and transferred to a large ceramic mortar on ice. 50 mL of ice-cold “homogenization buffer” (20 mM HEPES buffer, pH 7.0, with 1 mM MgCl₂) was added to the mortar and the tissue was homogenized with a pestle. The homogenate was centrifuged at 36,500 g for 20 minutes at 4° C. The supernatants were discarded and the pellets were re-suspended in homogenization buffer and centrifuged as before. The supernatants were again discarded and the pellets were re-suspended in 30 mL homogenization buffer and incubated in a 37° C. water bath for 20 minutes. The homogenate was then centrifuged as before. The supernatant was discarded and the membrane pellets were re-suspended in 30 mL “resuspension buffer” (50 mM Tris-HCl buffer, pH 7.4, containing 1 mM EDTA and 3 mM MgCl₂). A Bradford Protein assay was performed to determine protein concentration. The protein was aliquotted into screw cap Cryo tubes each containing ˜200 μL, flash frozen in liquid nitrogen and stored at −80° C. until used for the assay.

C. FAAH Mouse Brain Membrane (MBM) Homogenate Preparation.

Eighteen 10 to 12 week old female CD-1 mice housed at the animal care facility of Ironwood Pharmaceuticals were anesthetized by isoflurane anesthesia and rapidly decapitated using a small decapitator (Harvard Apparatus part #PY8 SS-0012, Holliston, Mass.). Whole brain tissue from these mice was collected (approximately 9.4 g total) and placed on aluminum foil sitting on dry ice to flash freeze the tissue. Tissue was thawed and used to prepare microsomes as described above for rat brain homogenates.

D. Determination of FAAH Activity

FAAH activity was assayed in the respective homogenates described herein (Rat brain, Mouse brain or Human brain) for certain exemplary substrate compounds using a modification of the method of Omeir et al. (1995 Life Sci 56:1999) and Fowler et al. (1997 J. Pharmacol Exp Ther 283:729). For assay of FAAH activity in rat brain membrane (RBM) homogenates, RBM homogenates (7 μg protein in 20 μL final volume of 10 mM Tris pH 6.5) were mixed with 180 μL of a mixture of the following: 2.0 μM unlabelled anandamide, 0.03 μCi radio labeled anandamide [ethanolamine 1-³H] (40-60 Cis/mmol, product number ART-626, American Radiolabeled Chemicals, St. Louis, Mo.), 1 mg/mL Bovine Serum Albumin (fatty acid-free BSA, electrophoresis grade, Sigma, St. Louis, Mo.), 10 mM Tris-HCl (pH 6.5), and 1 mM EDTA in the presence and absence of test compounds (vehicle was DMSO at a final concentration of 1%) and incubated for 10 minutes at 37° C. Samples were placed on ice to terminate the reactions.

³H-ethanolamine product and un-reacted ³H-anandamide substrate were then separated either: (1) by using chloroform/methanol extraction or (2) by passing the reaction mixture through a glass fiber filter containing activated charcoal. Samples were extracted with chloroform/methanol by adding 0.4 mL of chloroform/methanol (1:1 v/v), vigorously mixing the samples, and separating the aqueous and organic phases by centrifugation. Radioactivity (corresponding to FAAH-catalyzed breakdown of ³H-anandamide) found in aliquots (0.2 mL) of the aqueous phase was determined by liquid scintillation counting with quench correction. IC₅₀ values were determined as described by Jonsson et al. (2001 Br. J. Pharmacol. 133:1263). Alternatively, reactions were purified using a modification of the solid-phase extraction method described by Wilson et al (2003 Anal. Biochem. 318:270). This method was modified as follows: after reactions were incubated at 37° C. for 10 minutes and chilled on ice, the reaction mixtures were acidified by adding 10 μL of sodium phosphate solution [0.5M (pH 2.0)]. Next, 90 μL aliquots of the acidified reaction mixtures were applied to activated charcoal (that had been previously washed with methanol as described by Wilson et al. (supra)) containing 80 μL of water on top of a glass fiber filter, centrifuged, and the radioactivity in the eluate was counted as described previously by Wilson et al. (supra).

Table 1 provides activity data for certain compounds tested for inhibition of FAAH using the FAAH rat, mouse and human brain homogenate assays. The known FAAH inhibitors, 3′-(aminocarbonyl)biphenyl-3-yl cyclohexylcarbamate (URB597), [1-(4-chlorobenzoyl)-5-methoxy-2-methyl-1H-indol-3-yl]acetic acid (indomethacin) and 5-benzoyl-2,3-dihydro-1H-pyrrolizine-1-carboxylic acid (Ketorolac) were used as controls in these assays.

### Example 6

### Whole Cell Anandamide Hydrolysis Assay

FAAH activity was assayed in whole cells using methods disclosed previously (Maccarone et al. 1998 J. Biol. Chem. 273:32332 and Bisogno et al. 1997 J. Biol. Chem. 272:3315). In addition to the cell lines described in Maccarone et al. and Bisogno et al., MCF7 (ATCC designation HTB-22) and T84 (ATCC designation CCL-248) cell lines were also used in these assays.

A. Hela Cell Transfection with Human FAAH-1

cDNA expression clone for human FAAH-1 (in pcDNA3 vector) (Genbank Accession U82535; obtained from Benjamin Cravatt, Scripps Research Institute, La Jolla, Calif.) was linearized by digestion with BglII (New England Biolabs) and transfected by calcium phosphate into human HeLa cells (ATCC catalog #CCL-2). The HeLa cell line was selected as a host because it does not express FAAH or exhibit FAAH activity such that all subsequent activity can be attributed to the transfected gene. Following transfection, a stable HeLa-derived clone, designated 5c5, was isolated by single colony purification and expanded and maintained in modified Eagles medium (MEM; VWR catalog #45000-300) containing 10% fetal bovine serum (FBS), 2 mM L-glutamine, and 0.5 mg/mL G-418 (Sigma catalog #G5013).

B. FAAH Activity Assay

Clone 5c5 (50,000 cells in 150 μL) was seeded into 96-well plates and incubated overnight (5% CO₂, 37° C.). Media was carefully replaced with 180 μL DMEM/F12 medium (VWR catalog #45000-350) containing 15 mM HEPES, pH 7.4 and 0.1% fatty acid free BSA (Sigma catalog #A0281). Then, 2 μL of 100× desired final concentrations of certain exemplary compounds described herein were made up in DMSO, added to wells containing cells, and plates were incubated at 37° C. for 10 min. Next, 20 μL of 5 uM anandamide (Cayman catalog #90050) spiked with 8 uCi of anandamide-(ethanolamine-1-[³H]) (American Radiolabeled Chemicals, Inc., catalog #ART 626) was added to the cells and the plates were incubated for an additional 15 min at 37° C. The reactions were terminated by chilling the plates on ice and adding 20 μL of 0.5 M of potassium phosphate buffer (adjusted to pH 2.1 with phosphoric acid).

The acidified reactions were transferred to 96-well filter plates (0.25 mL capacity/well, 1.2 micron glass fiber pre-filter packed above 0.65 micron pore-size PVDF membrane, Millipore catalog MSFCN6B50) containing 25 μL charcoal (neutral activated carbon, Fisher Scientific catalog C170-500) per well. Prior to assay, charcoal was measured and loaded onto the plate using an aluminum 96-well column loading device (Millipore catalog MACL09625). The filter plate was assembled over empty 96-well plate (Costar) using a centrifuge alignment frame (Millipore catalog MACF09604) to allow collection of the filtrate in the receiver plate. The charcoal glass fiber filter plates were pre-washed with methanol by centrifugation 650×g for 10 min). Next, 80 μL of water was added to the wells of the pre-washed 96-well charcoal filter plate. Then, 90 μL of the acidified reaction mixture was added to the water in the wells of the charcoal plate. The samples were centrifuged as above. The substrate remained bound to the charcoal, whereas the [³H]-ethanolamine product formed flowed through and was transferred to the microplates containing scintillation cocktail and quantified in a micro-plate scintillation counter (Perkin-Elmer Microbeta). Control reactions with either no cells or cells treated with DMSO alone were performed in triplicate and used to define background (no cells) and 100% activity (DMSO alone).

Following subtraction of background radioactivity, data were expressed as percent inhibition relative to 100% activity and fit with a nonlinear regression curve using GraphPad Prism Software (GraphPad Software Inc). IC₅₀ values were calculated from the resulting dose-response curves constrained at top and bottom to 100% and 0%, respectively.

### Example 7

### Determination of Endogenous and Exogenous Anandamide Levels in Rat Plasma and Brain Tissue

The effects of some exemplary compounds described herein on endogenous and exogenously dosed anandamide (AEA) levels were measured. Rats dosed with test compound were sacrificed at various time points to determine the levels of anandamide both circulating and within the brain tissue. For experiments measuring exogenous levels of anandamide, the anandamide (Cayman Chemical, Ann Arbor, Mich. or Sigma Chemical, St. Louis, Mo.) was dosed (in the range of 3-30 mg/kg) post dosing of test compound. Animals were sacrificed at 5, 15, 30, or 60 minutes after anandamide administration with anesthesia administration followed by decapitation. Brains were removed immediately and the plasma was recovered from the blood for analysis of anandamide levels.

Flash frozen whole brain (e.g., from rat or mouse) samples were first transferred to clean 50 mL conical tubes and the wet brain weight was recorded. Fifteen mL of 9:1 ethyl acetate:hexane and 40 ng of deuterated anandamide (d8AEA) were added to the brain samples. The samples were then homogenized with an Omni GLH homogenizer until the solution was an uniform slurry, and 5.0 mL of water was added just prior to completion. Upon completion of the homogenization the tubes were held on ice. The chilled tubes were then vortexed and centrifuged at 4° C. at 3500 rpm for 10 minutes. One milliliter of the aqueous layer was sampled for use in a Bradford assay of protein content (Bradford, M. M. Anal. Biochem. 1976, 72: 248). The ethyl acetate layer was recovered, placed in a 15-mL glass tube, and evaporated under nitrogen in a TurboVac. Once dry, samples were reconstituted in 1 mL of 1:3 (v/v) CHCl₃:methanol and vortexed. Prepared brain samples were transferred to a 96-well plate for analysis by LC/MS/MS.

Stock standards were prepared at 0.0, 0.50, 1.0, 5.0, 10.0, 50.0, 100, 500, and 1000 ng/mL in methanol. Standards for LC/MS/MS were prepared with 0.5 mg Pefabloc, 10 μL of the stock standard to 90 μL of stock rat plasma and vortexing.

Frozen plasma samples containing pefabloc were thawed, and 100 μL of each sample was transferred to a microcentrifuge tube. To each standard and sample tube, 20 ng d8AEA and 100 μL of ice-cold acetone (for protein precipitation) were added. Tubes were vortexed, and centrifuged at 13,000 rpm for at least 5 minutes. The supernatants were collected in microcentrifuge tubes and the acetone was evaporated off in a TurboVac for 5-10 minutes. The evaporated supernatant solutions were next extracted with 250 μL of 1:2 (v/v) methanol:CHCl₃ and centrifuged at 13,000 rpm for at least 5 minutes. The CHCl₃ layer was collected and evaporated under nitrogen (TurboVac) until dry. Standards and samples were then reconstituted in 200 μL of 1:3 (v/v) CHCl₃:methanol. Prepared standards and plasma samples were transferred to a 96-well plate for analysis by LC/MS/MS. Similar experiments were performed using human plasma to which test compounds and exogenous anandamide was dosed or not dosed.

The LC/MS/MS method used a Waters 2777 sample manager, 1525 binary pump, and Quattro micro mass spectrometer. The separation was performed on a Waters Xterra MS C8, 5 μm, 2.1×20 mm analytical LC column with a Thermo Electron Javelin Basic 8, 2×10 mm guard column at a flow rate of 0.30 mL/min and a 25 μL injection volume. A binary linear gradient of mobile phase A (10 mM ammonium acetate in water (pH 9.5)) and mobile phase B (80:20 acetonitrile:methanol) was used from 2.0 to 2.2 minutes from 25% to 90% B, with a total run time of 6.0 minutes per sample injection. AEA and d8-AEA eluted in ˜3.5 minutes. The Quattro micro was operated in multiple reaction monitoring (MRM) mode with negative electrospray ionization. The mass transitions of 348 m/z-62 m/z (AEA) and 356 m/z-62 m/z (d8-AEA) were monitored using optimized collision settings (determined experimentally). Data were analyzed using Micromass QuanLynx software, and standard curves were generated using the ratio of the internal standard (d8-AEA) peak area to AEA peak area in response to AEA concentration. AEA concentration in brain and plasma samples was calculated using the linear regression of the standard curve. AEA concentration in plasma was reported as ng AEA/mL plasma, and AEA concentration in brain was reported as ng AEA/g protein (protein content determined by the Bradford assay).

The measured increase in endogenous AEA levels in rat brains for the tested compounds was generally between 0.7 and 3.3 fold.

TABLE 1
Average activity of the compounds of this invention, expressed as
IC₅₀ (the concentration of the agent needed to induce 50%
inhibition of the enzyme) of FAAH extracted from human, mouse
and rat brain homogenates.

| | Human brain | Mouse Brain | Rat Brian |
|---|---|---|---|
| | FAAH Extract | FAAH Extract | Extract |
| Compound # | IC₅₀ (μM) | IC₅₀ (μM) | IC₅₀ (μM) |
| I-01 | 0.542 | | |
| I-02 | 0.097 | | |
| I-03 | 0.094 | | |
| I-04 | 0.051 | | |
| I-05 | 0.004 | | |
| I-06 | 1.5 | | |
| I-07 | 0.018 | | |
| I-08 | 0.797 | >5.0 | |
| I-09 | 0.031 | 0.062 | |
| I-10 | 0.605 | >5.0 | |
| I-11 | 0.080 | 0.192 | |
| I-12 | 0.234 | 1.09 | |
| I-13 | 0.009 | 0.003 | |
| I-14 | 0.202 | 0.516 | |
| I-15 | 0.094 | 0.007 | |
| I-16 | 0.119 | 2.08 | 0.004 |
| I-17 | 1.22 | | |
| I-18 | 0.021 | 0.009 | 1.44 |
| I-19 | >5.0 | | 0.005 |
| I-20 | >5.0 | | 1.04 |
| I-21 | 1.01 | | 0.001 |
| I-22 | 4.11 | | 1.77 |
| I-23 | 2.56 | | 0.185 |
| I-24 | 50+ | | >5.0 |
| I-25 | 50+ | | 0.028 |
| I-26 | 50+ | | >5.0 |
| I-27 | 50+ | | |
| I-28 | 0.8969 | | |
| I-29 | 5.0 | | |
| I-30 | 3.4 | | |
| I-31 | 3.2 | | |
| I-32 | >5.0 | | |

1. A compound of Formula I, or a pharmaceutically acceptable salt thereof, 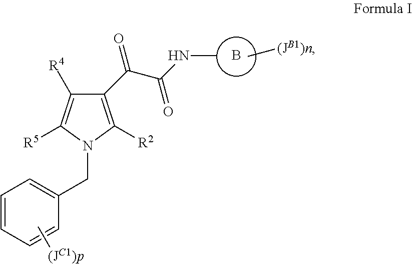
wherein:
ring B is selected from the group consisting of phenyl and a 5-6-membered monocyclic heteroaryl ring, wherein said monocyclic heteroaryl ring contains up to 3 ring heteroatoms selected from the group consisting of N, O and S;
n is an integer selected from the group consisting of 0, 1, 2 and 3;
each J^{B1} is independently selected from the group consisting of halogen, —NO₂, —CN, C₁₋₆ aliphatic, C₃₋₆ cycloaliphatic, C₁₋₆ haloaliphatic, C₁₋₆ alkoxy, C₁₋₆haloalkoxy and C₃₋₆ cycloalkoxy;
each J^{C1} is independently selected from the group consisting of halogen, —NO₂, —CN, C₁₋₆ aliphatic, C₃₋₆ cycloaliphatic, C₁₋₆ haloaliphatic, C₁₋₆ alkoxy, C₁₋₆haloalkoxy and C₃₋₆ cycloalkoxy;
p is an integer selected from the group consisting of 0, 1, 2 and 3;
R² is selected from the group consisting of halogen, —NO₂, —CN, C₁₋₆ aliphatic, phenyl, a 5-6 membered heteroaryl ring and a C₃₋₇ cycloalkyl, wherein said C₁₋₆ aliphatic, phenyl, 5-6 membered heteroaryl ring and C₃₋₇ cycloalkyl is optionally substituted by up to three instances of halogen;
R⁴ is selected from the group consisting of hydrogen, halogen, —CN, C₁₋₆ aliphatic, a C₃₋₇ cycloaliphatic ring, a 5-6 membered heteroaryl ring, phenyl, —OR^{Y} and —SR^{Y};
R⁵ is selected from the group consisting of hydrogen, halogen, —CN, C₁₋₆ aliphatic, a C₃₋₇ cycloaliphatic ring, a 5-6 membered heteroaryl ring, phenyl, —OR^{Y} and —SR^{Y}, wherein said C₁₋₆ aliphatic, C₃₋₇ cycloaliphatic ring, 5-6-membered heteroaryl ring, and phenyl is optionally substituted with up to three instances of halogen, C₁₋₄ alkyl, C₁₋₄ haloalkyl, C₁₋₄ alkoxy or C₁₋₄ haloalkoxy; or
R⁴ and R⁵, together with the two carbon atoms to which they are attached, form a C₅₋₈ cycloaliphatic ring, a 5-8-membered heterocyclic ring or a 5-membered heteroaryl ring; wherein said heterocyclic and heteroaryl ring formed by R⁴ and R⁵ contains up to three heteroatoms selected from the group consisting of N, O and S, and wherein said cycloaliphatic, heterocyclic and heteroaryl rings formed by R⁴ and R⁵ is optionally substituted by up to 3 instances of halogen, C₁₋₄ alkyl, C₁₋₄ haloalkyl, C₁₋₄ alkoxy or C₁₋₄ haloalkoxy; and
each R^{Y} is independently selected from the group consisting of C₁₋₆ aliphatic, C₃₋₇ cycloaliphatic, a 5-6-membered heteroaryl ring and phenyl, wherein each R^{Y} is optionally substituted by up to six instances of halogen, C₁₋₄ alkyl, C₁₋₄ haloalkyl, C₁₋₄ alkoxy or C₁₋₄ haloalkoxy;
provided that the compound is not:

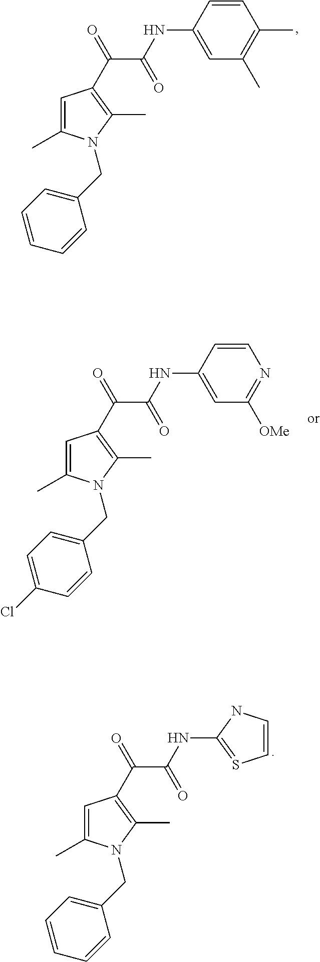

2. The compound according to claim 1, or a pharmaceutically acceptable salt thereof, wherein ring B is an optionally substituted ring selected from the group consisting of phenyl, pyridine, pyrimidine, pyrazine, pyridazine, pyrrole, imidazole, pyrazole, furan, thiophene, triazole, tetrazole, thiazole, oxathiazole and oxazole.

3. The compound according to claim 2, or a pharmaceutically acceptable salt thereof, wherein ring B is an optionally substituted pyridine or an optionally substituted phenyl.

4.-5. (canceled)

6. The compound according to claim 1, or a pharmaceutically acceptable salt thereof, wherein n is selected from the group consisting of 0 and 1.

7. The compound according to claim 1, or a pharmaceutically acceptable salt thereof, wherein each J^{B1} is independently selected from the group consisting of halogen, C₁₋₄ alkyl, cyclopropyl, cyclopropyloxy, C₁₋₄ haloalkyl, C₁₋₄ alkoxy and C₁₋₄ haloalkoxy.

8. The compound according to claim 7, or a pharmaceutically acceptable salt thereof, wherein each J^{B1} is independently selected from the group consisting of halogen, methyl, ethyl, propyl, isopropyl, trifluoromethyl, methoxy, trifluoromethoxy, ethoxy, propyloxy and isopropyloxy.

9. The compound according to claim 8, or a pharmaceutically acceptable salt thereof, wherein the moiety 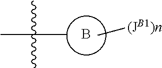
is selected from the group consisting of phenyl, 3-chlorophenyl, 3-pyridine, 4-pyridine and 3-methoxy-4-pyridine.

10. The compound according to claim 1, or a pharmaceutically acceptable salt thereof, wherein p is selected from the group consisting of 0, 1 and 2.

11. The compound according to claim 1, or a pharmaceutically acceptable salt thereof, wherein each J^{C1} is independently selected from the group consisting of halogen, C₁₋₄ alkyl, C₁₋₄ haloalkyl, cyclopropyl, cyclopropyloxy, C₁₋₄ alkoxy and C₁₋₄ haloalkoxy.

12. The compound according to claim 11, or a pharmaceutically acceptable salt thereof, wherein each J^{C1} is independently selected from the group consisting of halogen, methyl, ethyl, propyl, isopropyl, trifluoromethyl, methoxy, trifluoromethoxy, ethoxy, propyloxy and isopropyloxy.

13. The compound according to claim 12, or a pharmaceutically acceptable salt thereof, wherein each J^{C1} is halogen; J^{C1} is chlorine and p is 1 or 2; J^{C1} is fluorine and p is 1; or J^{C1} is methoxy and p is 1.

14.-16. (canceled)

17. The compound according to claim 1, or a pharmaceutically acceptable salt thereof, wherein R² is selected from the group consisting of halogen, —NO₂, —CN, C₁₋₆ aliphatic and phenyl, wherein each C₁₋₆ aliphatic and phenyl is optionally substituted with up to three instances of halogen.

18. The compound according to claim 17, or a pharmaceutically acceptable salt thereof, wherein R² is methyl, ethyl, propyl, isopropyl, butyl, isobutyl, t-butyl, pentyl or hexyl.

19.-20. (canceled)

21. The compound according to claim 1, or a pharmaceutically acceptable salt thereof, wherein R⁴ is hydrogen, C₁₋₄ alkyl, a 5-6-membered heteroaryl or phenyl.

22.-23. (canceled)

24. The compound according to claim 1, or a pharmaceutically acceptable salt thereof, wherein R⁵ is a C₁₋₄ alkyl, a 5-6-membered heteroaryl or phenyl.

25.-26. (canceled)

27. The compound according to claim 1, or a pharmaceutically acceptable salt thereof, wherein R⁴ and R⁵, together with the two carbon atoms to which they are attached, form a C₅₋₈ cycloaliphatic ring, a 5-8-membered heterocyclic ring or a 5-membered heteroaryl ring, wherein said cycloaliphatic, heterocyclic and heteroaryl ring formed by R⁴ and R⁵ is optionally substituted with up to 3 instances of halogen, C₁₋₂ alkyl, C₁₋₂ haloalkyl, C₁₋₂ alkoxy or C₁₋₂ haloalkoxy.

28. The compound according to claim 27, or a pharmaceutically acceptable salt thereof, wherein R⁴ and R⁵, together with the two carbon atoms to which they are attached, form an optionally substituted C₅₋₈ cycloaliphatic ring.

29. The compound according to claim 28, or a pharmaceutically acceptable salt thereof, wherein R⁴ and R⁵, together with the two carbon atoms to which they are attached, form the fused ring:

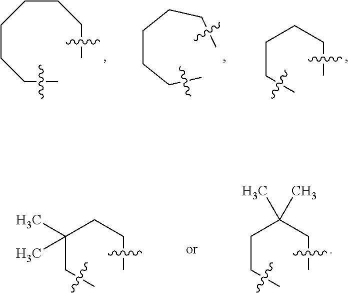

30. The compound according to claim 27, or a pharmaceutically acceptable salt thereof, wherein R⁴ and R⁵, together with the two carbon atoms to which they are attached, form an optionally substituted 5-membered heteroaryl ring.

31. The compound according to claim 30, or a pharmaceutically acceptable salt thereof, wherein R⁴ and R⁵, together with the two carbon atoms to which they are attached, form an optionally substituted thiophene ring.

32. The compound according to claim 31, or a pharmaceutically acceptable salt thereof, wherein R⁴ and R⁵, together with the pyrrole ring to which they are attached and its substituents, form

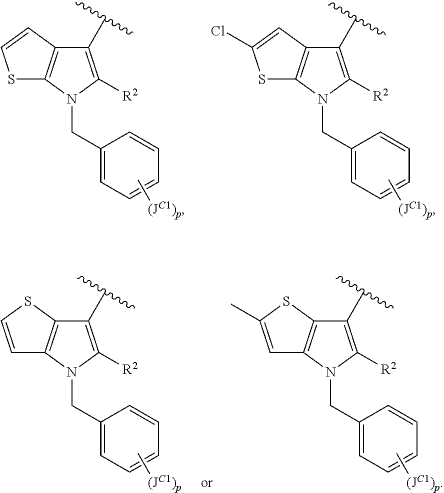

33. The compound according to claim 1, or a pharmaceutically acceptable salt thereof, represented by Formula II, 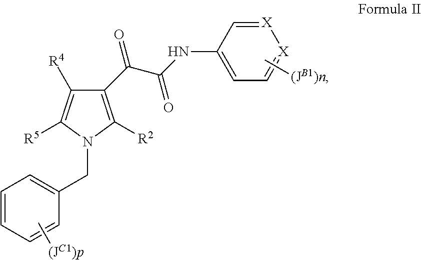
wherein each X is independently selected from the group consisting of C and N.

34. The compound according to claim 1, or a pharmaceutically acceptable salt thereof, represented by Formula III, 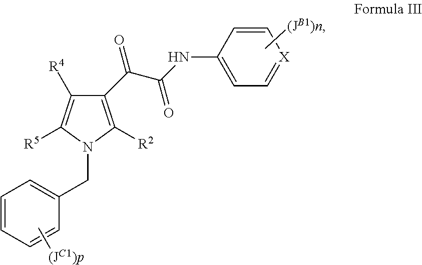
wherein: n is selected from the group consisting of 0 and 1 and J^{B1} is selected from the group consisting of halogen and methoxy.

35. The compound according to claim 1, represented by Formula IV, or a pharmaceutically acceptable salt thereof, 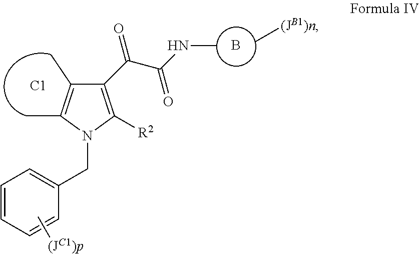
wherein ring C1 is an optionally substituted C₅₋₈ cycloaliphatic ring.

36. The compound according to claim 35, wherein ring C1 is optionally substituted with up to two instances of methyl.

37. The compound according to claim 1, represented by Formula V, or a pharmaceutically acceptable salt thereof, 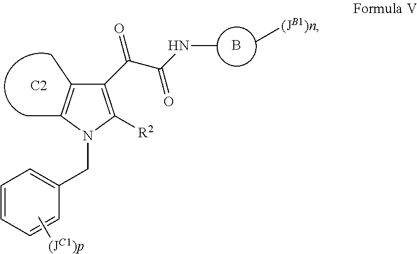
wherein ring C2 is an optionally substituted 5 membered heteroaryl ring.

38. The compound according to claim 37, or a pharmaceutically acceptable salt thereof, wherein ring C2 is an optionally substituted thiophene ring.

39. The compound according to claim 38, or a pharmaceutically acceptable salt thereof, wherein ring C2 is optionally substituted with up to two instances of methyl or halogen.

40. The compound according to claim 1, wherein the compound is:

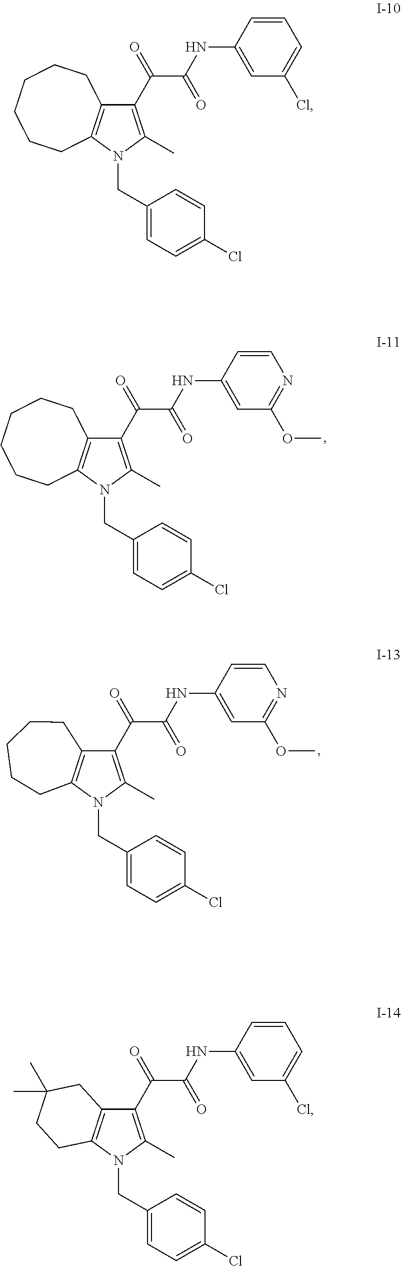

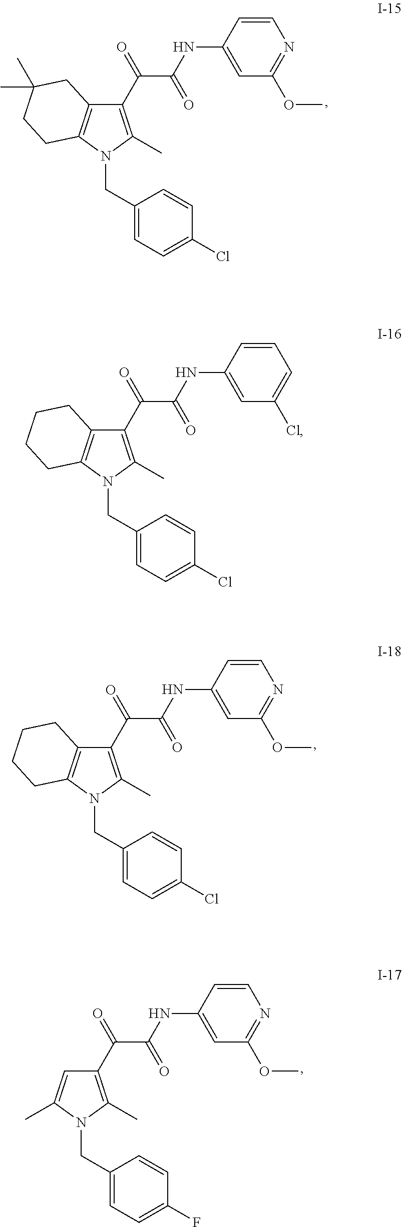

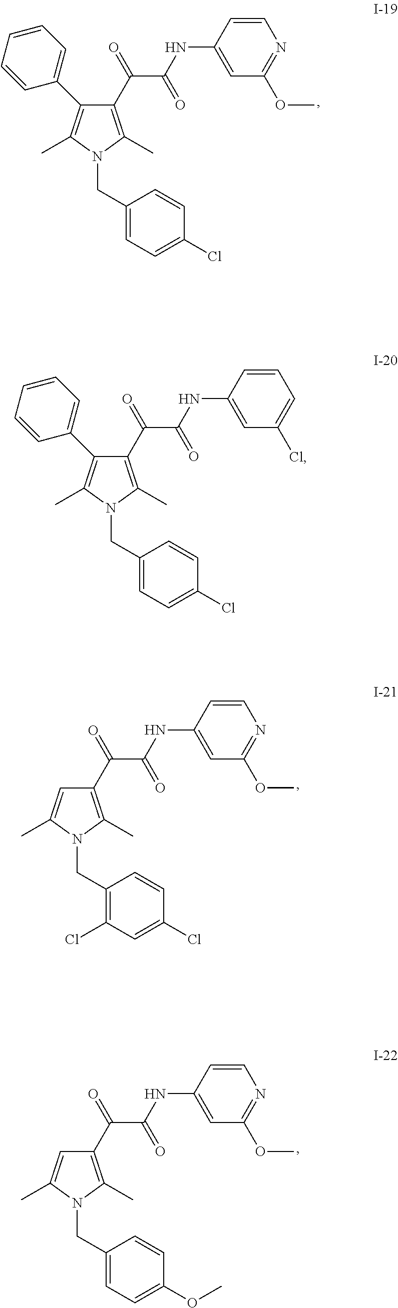

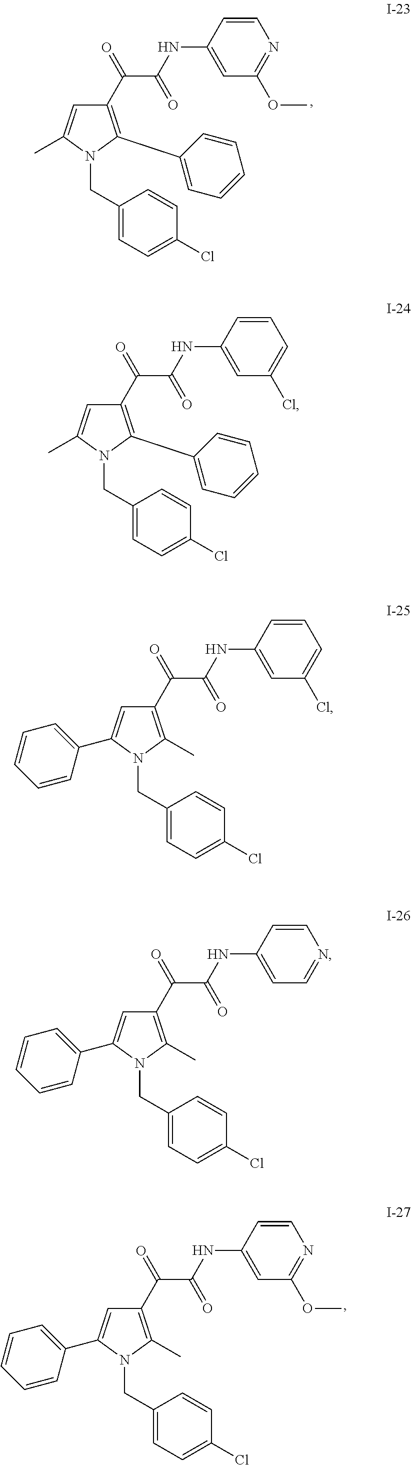

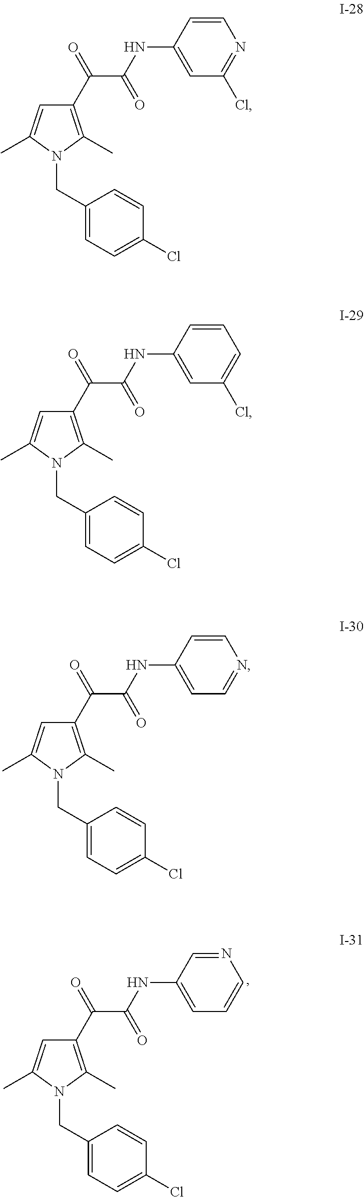 [SMILES: Cc1cc(C(=O)C(=O)Nc2ccnc(Cl)c2)c(C)n1Cc1ccc(Cl)cc1 ; Cc1cc(C(=O)C(=O)Nc2cccc(Cl)c2)c(C)n1Cc1ccc(Cl)cc1 ; Cc1cc(C(=O)C(=O)Nc2ccncc2)c(C)n1Cc1ccc(Cl)cc1 ; Cc1cc(C(=O)C(=O)Nc2cccnc2)c(C)n1Cc1ccc(Cl)cc1]

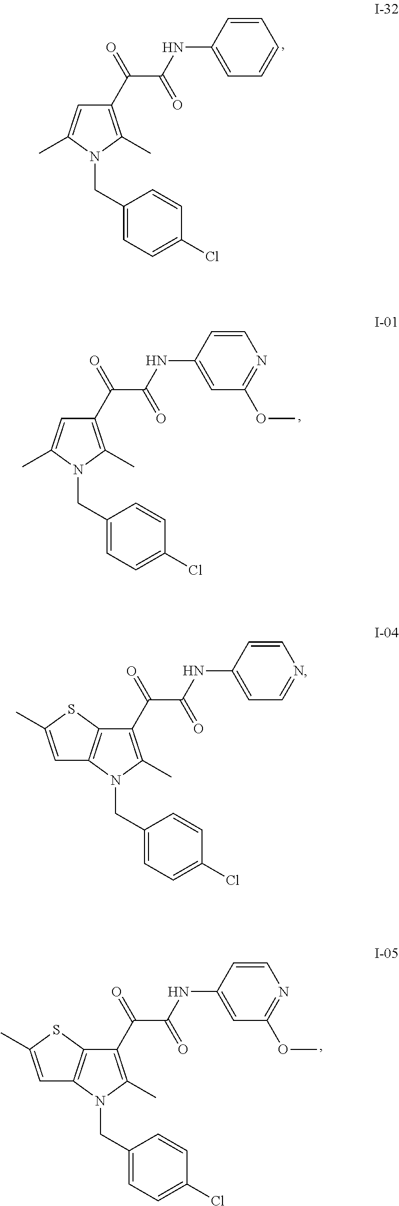

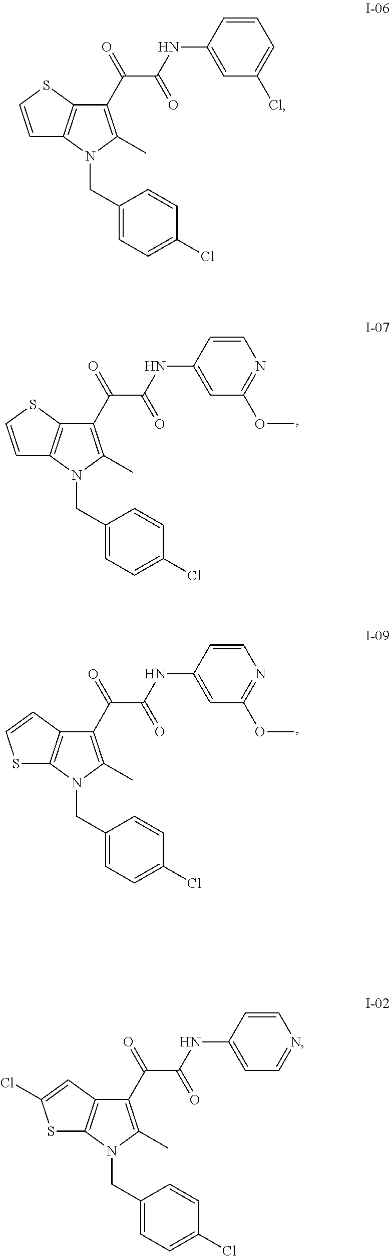 [SMILES: Cc1c(C(=O)C(=O)Nc2cccc(Cl)c2)c2sccc2n1Cc1ccc(Cl)cc1 ; COc1cc(NC(=O)C(=O)c2c(C)n(Cc3ccc(Cl)cc3)c3ccsc23)ccn1 ; COc1cc(NC(=O)C(=O)c2c(C)n(Cc3ccc(Cl)cc3)c3sccc23)ccn1 ; Cc1c(C(=O)C(=O)Nc2ccncc2)c2cc(Cl)sc2n1Cc1ccc(Cl)cc1]

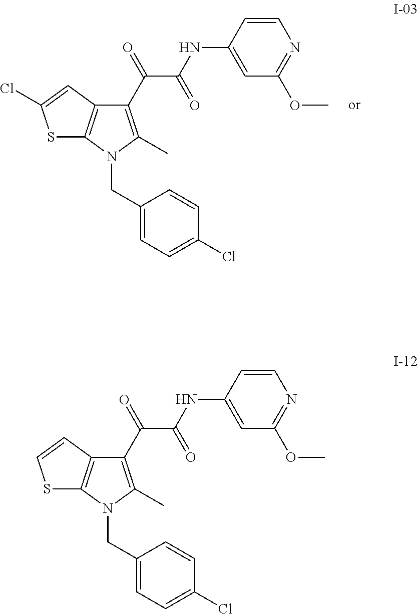 [SMILES: COc1cc(NC(=O)C(=O)c2c(C)n(Cc3ccc(Cl)cc3)c3sc(Cl)cc23)ccn1 ; COc1cc(NC(=O)C(=O)c2c(C)n(Cc3ccc(Cl)cc3)c3sccc23)ccn1]

41. A pharmaceutical composition comprising a compound according to claim 1, or a pharmaceutically acceptable salt thereof, and a pharmaceutically acceptable carrier, vehicle or adjuvant.

42. The pharmaceutical composition of claim 41, further comprising at least one additional therapeutic agent.

43. (canceled)

44. A method for the treatment or prevention of disorders selected from: pain; autoimmune disorders; disease states or indications that are accompanied by inflammatory processes; gastrointestinal diseases or disorders; pruritus; substance abuse-related syndromes, disorders, diseases or withdrawal symptoms; psychiatric disorders; neurological or neurodegenerative disorders; ocular disorders; appetite related disorders; gynecological disorders; or sleep disorders comprising administering, alone or in combination therapy, to a patient in need thereof a therapeutically or prophylactically acceptable dose of a pharmaceutical composition according to claim 41.

45.-70. (canceled)
